SEQUENCE LISTING (293 sequences)

SEQ ID NO: 1 - protein, 15 aa, Artificial Sequence
  CDR1
RASKGVSTSGYSYLH

SEQ ID NO: 2 - protein, 7 aa, Artificial Sequence
  CDR2
LASYLES

SEQ ID NO: 3 - protein, 9 aa, Artificial Sequence
  CDR3
QHSRDLPLT

SEQ ID NO: 4 - protein, 111 aa, Artificial Sequence [sequence omitted]

SEQ ID NO: 5 - protein, 218 aa, Artificial Sequence
  Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  CDR1
NYYMY

SEQ ID NO: 7 - protein, 17 aa, Artificial Sequence
  CDR2
GINPSNGGTNFNEKFKN

SEQ ID NO: 8 - protein, 11 aa, Artificial Sequence
  CDR3
RDYRFDMGFDY

SEQ ID NO: 9 - protein, 120 aa, Artificial Sequence [sequence omitted]

SEQ ID NO: 10 - protein, 447 aa, Artificial Sequence
  Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, Artificial Sequence
  CDR1
RASQSVSSYLA

SEQ ID NO: 12 - protein, 7 aa, Artificial Sequence
  CDR2
DASNRAT

SEQ ID NO: 13 - protein, 9 aa, Artificial Sequence
  CDR3
QQSSNWPRT

SEQ ID NO: 14 - protein, 107 aa, Artificial Sequence [sequence omitted]

SEQ ID NO: 15 - protein, 214 aa, Artificial Sequence
  Light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Artificial Sequence
  CDR1
NSGMH

SEQ ID NO: 17 - protein, 17 aa, Artificial Sequence
  CDR2
VIWYDGSKRYYADSVKG

SEQ ID NO: 18 - protein, 4 aa, Artificial Sequence
  CDR3
NDDY

SEQ ID NO: 19 - protein, 113 aa, Artificial Sequence [sequence omitted]

SEQ ID NO: 20 - protein, 440 aa, Artificial Sequence
  Heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, Artificial Sequence
  CDRL1
RASKSVSTSGFSYLH

SEQ ID NO: 22 - protein, 7 aa, Artificial Sequence
  CDRL2
LASNLES

SEQ ID NO: 23 - protein, 9 aa, Artificial Sequence
  CDRL3
QHSWELPLT

SEQ ID NO: 24 - protein, 5 aa, Artificial Sequence
  CDRH1
SYYLY

SEQ ID NO: 25 - protein, 17 aa, Artificial Sequence
  CDRH2
GVNPSNGGTNFSEKFKS

SEQ ID NO: 26 - protein, 11 aa, Artificial Sequence
  CDRH3
RDSNYDGGFDY

SEQ ID NO: 27 - protein, 120 aa, Artificial Sequence [sequence omitted]

SEQ ID NO: 28 - protein, 111 aa, Artificial Sequence [sequence omitted]

SEQ ID NO: 29 - protein, 111 aa, Artificial Sequence
  Light chain VR
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, Artificial Sequence
  Light chain VR
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, Artificial Sequence; identical to SEQ ID NO: 10
  Heavy chain

SEQ ID NO: 32 - protein, 218 aa, Artificial Sequence; identical to SEQ ID NO: 5
  Light Chain

SEQ ID NO: 33 - protein, 218 aa, Artificial Sequence
  Light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, Artificial Sequence
  Light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, Artificial Sequence
  CDR1
SDYWG

SEQ ID NO: 36 - protein, 16 aa, Artificial Sequence
  CDR2
FITYSGSTSYNPSLKS

SEQ ID NO: 37 - protein, 19 aa, Artificial Sequence
  CDR3
MPSFITLASLSTWEGYFDF

SEQ ID NO: 38 - protein, 11 aa, Artificial Sequence
  CDR1
KASQSIHKNLA

SEQ ID NO: 39 - protein, 7 aa, Artificial Sequence
  CDR2
YANSLQT

SEQ ID NO: 40 - protein, 8 aa, Artificial Sequence
  CDR3
QQYYSGWT

SEQ ID NO: 41 - protein, 127 aa, Artificial Sequence
  Parental VH
EVQLQESGPGLVKPSQSLSLTCSVTGSSIASDYWGWIRKFPGNKMEWMGFITYSGSTSYNPSLKSRISITRDTSKNQFFLQLHSVTTDDTATYSCARMPSFITLASLSTWEGYFDFWGPGTMVTVSS

SEQ ID NO: 42 - protein, 106 aa, Artificial Sequence
  Parental VL
DIQMTQSPSLLSASVGDRVTLNCKASQSIHKNLAWYQQKLGEAPKFLIYYANSLQTGIPSRFSGSGSGTDFTLTISGLQPEDVATYFCQQYYSGWTFGGGTKVELK

SEQ ID NO: 43 - protein, 127 aa, Artificial Sequence
  Variable heavy
EVQLQESGPGLVKPSETLSLTCTVSGGSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 44 - protein, 127 aa, Artificial Sequence
  variable heavy
EVQLQESGPGLVKPSETLSLTCTVSGGSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 45 - protein, 127 aa, Artificial Sequence
  variable heavy
EVQLQESGPGLVKPSETLSLTCTVSGGSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 46 - protein, 127 aa, Artificial Sequence
  variable heavy
EVQLQESGPGLVKPSETLSLTCTVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 47 - protein, 127 aa, Artificial Sequence
  variable heavy
EVQLQESGPGLVKPSETLSLTCTVSGGSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 48 - protein, 127 aa, Artificial Sequence
  variable heavy
EVQLQESGPGLVKPSETLSLTCTVSGGSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 49 - protein, 127 aa, Artificial Sequence
  variable heavy
EVQLQESGPGLVKPSETLSLTCTVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 50 - protein, 127 aa, Artificial Sequence
  variable heavy
EVQLQESGPGLVKPSETLSLTCTVSGSSISSDYWGWIRQPPGKGLEWMGFITYSGSTSYNPSLKSRITISVDTSKNQFSLKLHSVTAADTAVYYCARMPSFITLASLSTWEGYFDFWGQGTMVTVSS

SEQ ID NO: 51 - protein, 127 aa, Artificial Sequence
  variable heavy
EVQLQESGPGLVKPSETLSLTCAVSGYSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 52 - protein, 127 aa, Artificial Sequence
  variable heavy
EVQLQESGPGLVKPSETLSLTCAVSGYSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 53 - protein, 127 aa, Artificial Sequence
  variable heavy
EVQLQESGPGLVKPSETLSLTCAVSGYSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 54 - protein, 127 aa, Artificial Sequence
  variable heavy
EVQLQESGPGLVKPSETLSLTCAVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 55 - protein, 127 aa, Artificial Sequence
  variable heavy
EVQLQESGPGLVKPSETLSLTCAVSGYSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 56 - protein, 127 aa, Artificial Sequence
  variable heavy
EVQLQESGPGLVKPSETLSLTCAVSGYSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 57 - protein, 127 aa, Artificial Sequence
  variable heavy
EVQLQESGPGLVKPSETLSLTCAVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 58 - protein, 127 aa, Artificial Sequence
  variable heavy
EVQLQESGPGLVKPSETLSLTCAVSGSSISSDYWGWIRQPPGKGLEWMGFITYSGSTSYNPSLKSRITISRDTSKNQFSLKLHSVTAADTAVYYCARMPSFITLASLSTWEGYFDFWGQGTMVTVSS

SEQ ID NO: 59 - protein, 106 aa, Artificial Sequence
  variable light
DIQMTQSPSSLSASVGDRVTITCKASQSIHKNLAWYQQKPGKAPKLLIYYANSLQTGVPSRFSGSGSGTDFTLTISSLQPEDFATYYCQQYYSGWTFGGGTKVEIK

SEQ ID NO: 60 - protein, 106 aa, Artificial Sequence
  variable light
DIQMTQSPSSLSASVGDRVTITCKASQSIHKNLAWYQQKPGKAPKFLIYYANSLQTGVPSRFSGSGSGTDFTLTISSLQPEDFATYYCQQYYSGWTFGGGTKVEIK

SEQ ID NO: 61 - protein, 106 aa, Artificial Sequence
  variable light
DIQMTQSPSSLSASVGDRVTITCKASQSIHKNLAWYQQKPGKAPKFLIYYANSLQTGIPSRFSGSGSGTDFTLTISSLQPEDFATYYCQQYYSGWTFGGGTKVEIK

SEQ ID NO: 62 - protein, 106 aa, Artificial Sequence
  variable light
DIQMTQSPSSLSASVGDRVTITCKASQSIHKNLAWYQQKPGKVPKLLIYYANSLQTGVPSRFSGSGSGTDFTLTISSLQPEDVATYYCQQYYSGWTFGGGTKVEIK

SEQ ID NO: 63 - protein, 106 aa, Artificial Sequence
  variable light
DIQMTQSPSSLSASVGDRVTITCKASQSIHKNLAWYQQKPGKVPKFLIYYANSLQTGVPSRFSGSGSGTDFTLTISSLQPEDVATYYCQQYYSGWTFGGGTKVEIK

SEQ ID NO: 64 - protein, 106 aa, Artificial Sequence
  variable light
DIQMTQSPSSLSASVGDRVTITCKASQSIHKNLAWYQQKPGKVPKFLIYYANSLQTGIPSRFSGSGSGTDFTLTISSLQPEDVATYYCQQYYSGWTFGGGTKVEIK

SEQ ID NO: 65 - protein, 129 aa, Artificial Sequence
  variable heavy
EVQLQESGPGLVKPSETLSLTCTVSGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSSAS

SEQ ID NO: 66 - protein, 127 aa, Artificial Sequence
  variable heavy
EVQLQESGPGLVKPSETLSLTCTVSGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 67 - protein, 127 aa, Artificial Sequence
  variable heavy
EVQLQESGPGLVKPSETLSLTCTVSGSSIASDYWGWIRKPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 68 - protein, 127 aa, Artificial Sequence
  variable heavy
EVQLQESGPGLVKPSETLSLTCTVSGSSIASDYWGWIRQPPGKKLEWIGFITYSGSTSYNPSLKSRVTISRDTSKNQFSLKLSSVTAADTAVYYCARMPSFITLASLSTWEGYFDFWGQGTMVTVSS

SEQ ID NO: 69 - protein, 127 aa, Artificial Sequence
  variable heavy
EVQLQESGPGLVKPSETLSLTCTVSGSSIASDYWGWIRQPPGKGMEWIGFITYSGSTSYNPSLKSRVTISRDTSKNQFSLKLSSVTAADTAVYYCARMPSFITLASLSTWEGYFDFWGQGTMVTVSS

SEQ ID NO: 70 - protein, 127 aa, Artificial Sequence
  variable heavy
EVQLQESGPGLVKPSETLSLTCTVSGSSIASDYWGWIRKPPGKKMEWIGFITYSGSTSYNPSLKSRVTISRDTSKNQFSLKLSSVTAADTAVYYCARMPSFITLASLSTWEGYFDFWGQGTMVTVSS

SEQ ID NO: 71 - protein, 127 aa, Artificial Sequence
  variable heavy
EVQLQESGPGLVKPSETLSLTCTVSGSSIASDYWGWIRQFPGKGLEWIGFITYSGSTSYNPSLKSRVTISRDTSKNQFSLKLSSVTADDTAVYYCARMPSFITLASLSTWEGYFDFWGQGTMVTVSS

SEQ ID NO: 72 - protein, 127 aa, Artificial Sequence
  variable heavy
EVQLQESGPGLVKPSETLSLTCTVSGSSIASDYWGWIRKPPGKKMEWIGFITYSGSTSYNPSLKSRVTISVDTSKNQFSLKLSSVTAADTAVYYCARMPSFITLASLSTWEGYFDFWGQGTMVTVSS

SEQ ID NO: 73 - protein, 127 aa, Artificial Sequence
  variable heavy
EVQLQESGPGLVKPSETLSLTCSVTGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 74 - protein, 127 aa, Artificial Sequence
  variable heavy
EVQLQQSGAGLLKPSETLSLTCSVTGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 75 - protein, 127 aa, Artificial Sequence
  variable heavy
EVQLQESGPGLVKPPGTLSLTCSVTGSSIASDYWGWVRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 76 - protein, 106 aa, Artificial Sequence; identical to SEQ ID NO: 59
  variable light

SEQ ID NO: 77 - protein, 106 aa, Artificial Sequence; identical to SEQ ID NO: 60
  variable light

SEQ ID NO: 78 - protein, 106 aa, Artificial Sequence
  variable light
DIQMTQSPSSLSASVGDRVTITCKASQSIHKNLAWYQQKPGKAPKLLIYYANSLQTGVPSRFSGSGSGTDFTLTISSLQPEDFATYFCQQYYSGWTFGGGTKVEIK

SEQ ID NO: 79 - protein, 106 aa, Artificial Sequence
  variable light
DIQMTQSPSSLSASVGDRVTITCKASQSIHKNLAWYQQKPGKAPKFLIYYANSLQTGVPSRFSGSGSGTDFTLTISSLQPEDFATYFCQQYYSGWTFGGGTKVEIK

SEQ ID NO: 80 - protein, 106 aa, Artificial Sequence
  variable light
DIQMTQSPSSLSASVGDRVTITCKASQSIHKNLAWYQQKPGKAPKLLIYYANSLQTGIPSRFSGSGSGTDFTLTISSLQPEDFATYYCQQYYSGWTFGGGTKVEIK

SEQ ID NO: 81 - protein, 11 aa, Artificial Sequence
  CDR1
GYSITSDYAWN

SEQ ID NO: 82 - protein, 16 aa, Artificial Sequence
  CDR2
YISNSGSASYNPSLKS

SEQ ID NO: 83 - protein, 12 aa, Artificial Sequence
  CDR3
LIYYDYGGAMNF

SEQ ID NO: 84 - protein, 11 aa, Artificial Sequence
  CDR1
KASQGVSTTVA

SEQ ID NO: 85 - protein, 7 aa, Artificial Sequence
  CDR2
SASYRYT

SEQ ID NO: 86 - protein, 9 aa, Artificial Sequence
  CDR3
QHYYSTPWT

SEQ ID NO: 87 - protein, 121 aa, Artificial Sequence
  parental VH
DVQLQESGPGLVKPSQSLSLTCTVTGYSITSDYAWNWIRQFPGNKLEWMGYISNSGSASYNPSLKSRISITRDTSKNQFFLQLNSVTTEDTATYYCATLIYYDYGGAMNFWGQGTSVTVSS

SEQ ID NO: 88 - protein, 107 aa, Artificial Sequence
  parental VL
DIVMTQSHKFMSTSVGDRVSITCKASQGVSTTVAWYQQKPGQSPKLLIYSASYRYTGVPDRFTGSGSGTDFTFTISSVQSEDLAVYYCQHYYSTPWTFGGGTKLEIK

SEQ ID NO: 89 - protein, 7 aa, Artificial Sequence
  CDR2 variant
YASNLQT

SEQ ID NO: 90 - protein, 7 aa, Artificial Sequence
  CDR2 variant
YASSLQT

SEQ ID NO: 91 - protein, 7 aa, Artificial Sequence
  CDR2 variant
YASTLQT

SEQ ID NO: 92 - protein, 7 aa, Artificial Sequence
  CDR2 variant
YATTLQT

SEQ ID NO: 93 - protein, 7 aa, Artificial Sequence
  CDR2 variant
YASYLQT

SEQ ID NO: 94 - protein, 7 aa, Artificial Sequence
  CDR2 variant
YANQLQT

SEQ ID NO: 95 - protein, 7 aa, Artificial Sequence
  CDR2 variant
YAGSLQT

SEQ ID NO: 96 - protein, 7 aa, Artificial Sequence
  CDR2 variant
YASQLQT

SEQ ID NO: 97 - protein, 7 aa, Artificial Sequence
  CDR2 variant
YADSLQT

SEQ ID NO: 98 - protein, 8 aa, Artificial Sequence
  CDR3 variant
QQYYSGFT

SEQ ID NO: 99 - protein, 8 aa, Artificial Sequence
  CDR3 variant
QQYYSGYT

SEQ ID NO: 100 - protein, 8 aa, Artificial Sequence
  CDR3 variant
QQYYSGIT

SEQ ID NO: 101 - protein, 8 aa, Artificial Sequence
  CDR3 variant
QQYYSGVT

SEQ ID NO: 102 - protein, 8 aa, Artificial Sequence
  CDR3 variant
QQYYSGLT

SEQ ID NO: 103 - protein, 19 aa, Artificial Sequence
  CDR3 variant
MPSFITLASLSTFEGYFDF

SEQ ID NO: 104 - protein, 19 aa, Artificial Sequence
  CDR3 variant
MPSFITLASLSTYEGYFDF

SEQ ID NO: 105 - protein, 19 aa, Artificial Sequence
  CDR3 variant
MPSFITLASLSTIEGYFDF

SEQ ID NO: 106 - protein, 19 aa, Artificial Sequence
  CDR3 variant
MPSFITLASLSTVEGYFDF

SEQ ID NO: 107 - protein, 19 aa, Artificial Sequence
  CDR3 variant
MPSFITLASLSTLEGYFDF

SEQ ID NO: 108 - protein, 5 aa, Artificial Sequence
  CDR1
SYVMH

SEQ ID NO: 109 - protein, 17 aa, Artificial Sequence
  CDR2
YIDPYNDGAKYNEKFKG

SEQ ID NO: 110 - protein, 10 aa, Artificial Sequence
  CDR3
GGPYGWYFDV

SEQ ID NO: 111 - protein, 11 aa, Artificial Sequence
  CDR1
RASEHIYSYLS

SEQ ID NO: 112 - protein, 7 aa, Artificial Sequence
  CDR2
NAKTLAE

SEQ ID NO: 113 - protein, 9 aa, Artificial Sequence
  CDR3
QHHFGSPLT

SEQ ID NO: 114 - protein, 119 aa, Artificial Sequence
  parental VH
EVQLQQSGPELVKPGSSVKMSCKASGYTFSSYVMHWVKQKPGQGLEWIGYIDPYNDGAKYNEKFKGKATLTSDKSSSTAYMELSSLTSEDSAVYYCARGGPYGWYFDVWGAGTTVTVSS

SEQ ID NO: 115 - protein, 107 aa, Artificial Sequence
  parental VL
DIQMTQSPASLSASVGETVTITCRASEHIYSYLSWYQQKQGKSPQLLVYNAKTLAEGVPSRFSGSGSGTQFSLKINSLQPEDFGTYYCQHHFGSPLTFGAGTTLELK

SEQ ID NO: 116 - protein, 16 aa, Artificial Sequence
  CDRH2 variant
YIDPYNRGAKYNEKFG

SEQ ID NO: 117 - protein, 16 aa, Artificial Sequence
  CDRH2 variant
YIDPYNLGAKYNEKFG

SEQ ID NO: 118 - protein, 16 aa, Artificial Sequence
  CDRH2 variant
YIDPYNKGAKYNEKFG

SEQ ID NO: 119 - protein, 16 aa, Artificial Sequence
  CDRH2 variant
YIDPYNFGAKYNEKFG

SEQ ID NO: 120 - protein, 16 aa, Artificial Sequence
  CDRH2 variant
YIDPYNSGAKYNEKFG

SEQ ID NO: 121 - protein, 16 aa, Artificial Sequence
  CDRH2 variant
YIDPYNYGAKYNEKFG

SEQ ID NO: 122 - protein, 16 aa, Artificial Sequence
  CDRH2 variant
YIDPYNVGAKYNEKFG

SEQ ID NO: 123 - protein, 17 aa, Artificial Sequence
  CDRH2 variant
YIDPYNDRAKYNEKFKG

SEQ ID NO: 124 - protein, 17 aa, Artificial Sequence
  CDRH2 variant
YIDPYNDNAKYNEKFKG

SEQ ID NO: 125 - protein, 17 aa, Artificial Sequence
  CDRH2 variant
YIDPYNDQAKYNEKFKG

SEQ ID NO: 126 - protein, 17 aa, Artificial Sequence
  CDRH2 variant
YIDPYNDEAKYNEKFKG

SEQ ID NO: 127 - protein, 17 aa, Artificial Sequence
  CDRH2 variant
YIDPYNDLAKYNEKFKG

SEQ ID NO: 128 - protein, 17 aa, Artificial Sequence
  CDRH2 variant
YIDPYNDKAKYNEKFKG

SEQ ID NO: 129 - protein, 17 aa, Artificial Sequence
  CDRH2 variant
YIDPYNDSAKYNEKFKG

SEQ ID NO: 130 - protein, 17 aa, Artificial Sequence
  CDRH2 variant
YIDPYNDYAKYNEKFKG

SEQ ID NO: 131 - protein, 17 aa, Artificial Sequence
  CDRH2 variant
YIDPYNDVAKYNEKFKG

SEQ ID NO: 132 - protein, 7 aa, Artificial Sequence
  CDRL2 variant
AAKTLAE

SEQ ID NO: 133 - protein, 7 aa, Artificial Sequence
  CDRL2 variant
YAKTLAE

SEQ ID NO: 134 - protein, 7 aa, Artificial Sequence
  CDRL2 variant
WAKTLAE

SEQ ID NO: 135 - protein, 7 aa, Artificial Sequence
  CDRL2 variant
SAKTLAE

SEQ ID NO: 136 - protein, 7 aa, Artificial Sequence
  CDRL2 variant
TAKTLAE

SEQ ID NO: 137 - protein, 7 aa, Artificial Sequence
  CDRL2 variant
IAKTLAE

SEQ ID NO: 138 - protein, 7 aa, Artificial Sequence
  CDRL2 variant
VAKTLAE

SEQ ID NO: 139 - protein, 7 aa, Artificial Sequence
  CDRL2 variant
NNKTLAE

SEQ ID NO: 140 - protein, 7 aa, Artificial Sequence
  CDRL2 variant
NIKTLAE

SEQ ID NO: 141 - protein, 7 aa, Artificial Sequence
  CDRL2 variant
NLLTLAE

SEQ ID NO: 142 - protein, 7 aa, Artificial Sequence
  CDRL2 variant
NTKTLAE

SEQ ID NO: 143 - protein, 7 aa, Artificial Sequence
  CDRL2 variant
NVKTLAE

SEQ ID NO: 144 - protein, 119 aa, Artificial Sequence
  variable heavy
EVQLVQSGAEVKKPGASVKVSCKASGYTFSSYVMHWVRQAPGQRLEWIGYIDPYNDGAKYSQKFQGRVTLTRDTSASTAYMELSSLRSEDTAVYYCARGGPYGWYFDVWGQGTTVTVSS

SEQ ID NO: 145 - protein, 119 aa, Artificial Sequence
  variable heavy
EVQLVQSGAEVKKPGASVKVSCKASGYTFSSYVMHWVRQAPGQRLEWIGYIDPYNDGAKYSQKFQGRVTLTSDKSASTAYMELSSLRSEDTAVYYCARGGPYGWYFDVWGQGTTVTVSS

SEQ ID NO: 146 - protein, 119 aa, Artificial Sequence
  variable heavy
EVQLVQSGAEVKKPGASVKVSCKASGYTFSSYVMHWVRQAPGQGLEWIGYIDPYNDGAKYAQKFQGRVTLTRDTSTSTVYMELSSLRSEDTAVYYCARGGPYGWYFDVWGQGTTVTVSS

SEQ ID NO: 147 - protein, 119 aa, Artificial Sequence
  variable heavy
EVQLVQSGAEVKKPGASVKVSCKASGYTFSSYVMHWVRQAPGQGLEWIGYIDPYNDGAKYAQKFQGRVTLTSDKSTSTVYMELSSLRSEDTAVYYCARGGPYGWYFDVWGQGTTVTVSS

SEQ ID NO: 148 - protein, 119 aa, Artificial Sequence
  variable heavy
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 149 - protein, 119 aa, Artificial Sequence
  variable heavy
EVQLVQSGAEVKKPGASVKVSCKASGYTFSSYVMHWVRQAPGQGLEWIGYIDPYNDGAKYAQKFQGRVTLTSDKSISTAYMELSRLRSDDTVVYYCARGGPYGWYFDVWGQGTTVTVSS

SEQ ID NO: 150 - protein, 107 aa, Artificial Sequence
  variable light
DIQMTQSPSSLSASVGDRVTITCRASEHIYSYLSWYQQKPGKAPKLLIYNAKTLAEGVPSRFSGSGSGTDFTLTISSLQPEDFATYYCQHHFGSPLTFGQGTRLEIK

SEQ ID NO: 151 - protein, 107 aa, Artificial Sequence
  variable light
DIQMTQSPSSLSASVGDRVTITCRASEHIYSYLSWYQQKPGKAPKLLIYNAKTLAEGVPSRFSGSGSGTQFTLTISSLQPEDFATYYCQHHFGSPLTFGQGTRLEIK

SEQ ID NO: 152 - protein, 107 aa, Artificial Sequence
  variable light
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 153 - protein, 107 aa, Artificial Sequence
  variable light
DIQMTQSPSSLSASVGDRVTITCRASEHIYSYLSWYQQKPGKVPKLLIYNAKTLAEGVPSRFSGSGSGTQFTLTISSLQPEDVATYYCQHHFGSPLTFGQGTRLEIK

SEQ ID NO: 154 - protein, 17 aa, Artificial Sequence
  CDR2 variant
YIDPYNDGAKYAQKFQG

SEQ ID NO: 155 - protein, 17 aa, Artificial Sequence
  CDR2 variant
YIDPYNDGAKYSQKFQG

SEQ ID NO: 156 - protein, 116 aa, Artificial Sequence
  variable heavy
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSS

SEQ ID NO: 157 - protein, 106 aa, Artificial Sequence
  variable light
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELK

SEQ ID NO: 158 - protein, 119 aa, Artificial Sequence
  variable heavy
EVQLVESGGDLVQPGRSLKISCVASGFTFSDYYMAWVRLAPQKGLEWVASISYEGSRTHYGDSVRGRFIISRDNPKNILYLQMNSLGSEDTATYFCARHTGTLDWLVYWGQGTLVIVSS

SEQ ID NO: 159 - protein, 106 aa, Artificial Sequence
  variable light
NIVMAQSPKSMSISAGDRVTMNCKASQNVDNNIAWYQQKPGQSPKLLIFYASNRYSGVPDRFTGGGYGTDFTLTIKSVQAEDAAFYYCQRIYNFPTFGSGTKLEIK

SEQ ID NO: 160 - protein, 19 aa, Artificial Sequence
  CDR3 consensus
  MISC_FEATURE (13)..(13): X is W, F, Y, I, V or L
MPSFITLASLSTXEGYFDF

SEQ ID NO: 161 - protein, 7 aa, Artificial Sequence
  CDR2 consensus
  MISC_FEATURE (3)..(3): X is N, S, T, G or D
  MISC_FEATURE (4)..(4): X is S, N, S, T, Y or Q
YAXXLQT

SEQ ID NO: 162 - protein, 8 aa, Artificial Sequence
  CDR3 consensus
  MISC_FEATURE (7)..(7): X is W, F, Y, I, V, OR L
QQYYSGXT

SEQ ID NO: 163 - protein, 127 aa, Artificial Sequence
  VH parental
  MISC_FEATURE (6)..(6): X is E or Q
  MISC_FEATURE (9)..(9): X is P or A
  MISC_FEATURE (12)..(12): X is V or L
  MISC_FEATURE (15)..(15): X is S or P
  MISC_FEATURE (16)..(16): X is Q, E, or G
  MISC_FEATURE (17)..(17): X is S or T
  MISC_FEATURE (23)..(23): X is S, T, or A
  MISC_FEATURE (25)..(25): X is T or S
  MISC_FEATURE (27)..(27): X is S, G, or Y
  MISC_FEATURE (30)..(30): X is A or S
  MISC_FEATURE (37)..(37): X is I or V
  MISC_FEATURE (39)..(39): X is K or Q
  MISC_FEATURE (40)..(40): X is F or P
  MISC_FEATURE (43)..(43): X is N or K
  MISC_FEATURE (44)..(44): X is K or G
  MISC_FEATURE (45)..(45): X is M or L
  MISC_FEATURE (48)..(48): X is M or I
  MISC_FEATURE (67)..(67): X is I or V
  MISC_FEATURE (68)..(68): X is S or T
  MISC_FEATURE (70)..(70): X is T or S
  MISC_FEATURE (71)..(71): X is R or V
  MISC_FEATURE (79)..(79): X is F or S
  MISC_FEATURE (81)..(81): X is Q or K
  MISC_FEATURE (83)..(83): X is H or S
  MISC_FEATURE (87)..(87): X is T or A
  MISC_FEATURE (88)..(88): X is D or A
  MISC_FEATURE (92)..(92): X is T or V
  MISC_FEATURE (94)..(94): X is S or Y
  MISC_FEATURE (110)..(110): X is W, F, Y, I, V, or L
  MISC_FEATURE (119)..(119): X is P or Q
  MISC_FEATURE (122)..(122): X is M, V, L, A, R, N, P, Q, E, G, I, H, K, F, S, T, W or Y
  MISC_FEATURE (123)..(123): X is V, T, or L
EVQLQXSGXGLXKPXXXLSLTCXVXGXSIXSDYWGWXRXXPGXXXEWXGFITYSGSTSYNPSLKSRXXIXXDTSKNQFXLXLXSVTXXDTAXYXCARMPSFITLASLSTXEGYFDFWGXGTXXTVSS

SEQ ID NO: 164 - protein, 127 aa, Artificial Sequence
  Variable heavy consensus
  MISC_FEATURE (6)..(6): X is E or Q
  MISC_FEATURE (9)..(9): X is P or A
  MISC_FEATURE (12)..(12): X is V or L
  MISC_FEATURE (15)..(15): X is S or P
  MISC_FEATURE (16)..(16): X is E or G
  MISC_FEATURE (23)..(23): X is T, A, or S
  MISC_FEATURE (25)..(25): X is S or T
  MISC_FEATURE (27)..(27): X is G, S, or Y
  MISC_FEATURE (30)..(30): X is S or A
  MISC_FEATURE (37)..(37): X is I or V
  MISC_FEATURE (39)..(39): X is Q or K
  MISC_FEATURE (40)..(40): X is P or F
  MISC_FEATURE (44)..(44): X is G or K
  MISC_FEATURE (45)..(45): X is L or M
  MISC_FEATURE (48)..(48): X is I or M
  MISC_FEATURE (67)..(67): X is V or I
  MISC_FEATURE (71)..(71): X is V or R
  MISC_FEATURE (83)..(83): X is S or H
  MISC_FEATURE (88)..(88): X is A or D
  MISC_FEATURE (110)..(110): X is W, F, Y, I, V, or L
  MISC_FEATURE (122)..(122): X is M, V, L, A, R, N, P, Q, E, G, I, H, K, F, S, T, W, or Y
  MISC_FEATURE (123)..(123): X is V, T, or L
EVQLQXSGXGLXKPXXTLSLTCXVXGXSIXSDYWGWXRXXPGKXXEWXGFITYSGSTSYNPSLKSRXTISXDTSKNQFSLKLXSVTAXDTAVYYCARMPSFITLASLSTXEGYFDFWGQGTXXTVSS

SEQ ID NO: 165 - protein, 106 aa, Artificial Sequence
  Variable light parental consensus
  MISC_FEATURE (10)..(10): X is L or S
  MISC_FEATURE (21)..(21): X is L or I
  MISC_FEATURE (22)..(22): X is N or T
  MISC_FEATURE (40)..(40): X is L or P
  MISC_FEATURE (42)..(42): X is E or K
  MISC_FEATURE (43)..(43): X is A or V
  MISC_FEATURE (46)..(46): X is F or L
  MISC_FEATURE (52)..(52): X is N, S, T, G, or D
  MISC_FEATURE (53)..(53): X is S, N, T, Y, or Q
  MISC_FEATURE (58)..(58): X is I or V
  MISC_FEATURE (77)..(77): X is G or S
  MISC_FEATURE (83)..(83): X is V or F
  MISC_FEATURE (87)..(87): X is F or Y
  MISC_FEATURE (95)..(95): X is W, F, Y, I, V, or L
  MISC_FEATURE (105)..(105): X is L or I
DIQMTQSPSXLSASVGDRVTXXCKASQSIHKNLAWYQQKXGXXPKXLIYYAXXLQTGXPSRFSGSGSGTDFTLTISXLQPEDXATYXCQQYYSGXTFGGGTKVEXK

SEQ ID NO: 166 - protein, 106 aa, Artificial Sequence
  variable light consensus
  misc_feature (43)..(43): X is A or V
  misc_feature (46)..(46): X is L or F
  misc_feature (52)..(52): X is N, S, T, G, or D
  MISC_FEATURE (53)..(53): X is S, N, T, Y, or Q
  MISC_FEATURE (58)..(58): X is V or I
  MISC_FEATURE (83)..(83): X is F or V
  MISC_FEATURE (95)..(95): X is W, F, Y, I, V, or L
DIQMTQSPSSLSASVGDRVTITCKASQSIHKNLAWYQQKPGKXPKXLIYYAXXLQTGXPSRFSGSGSGTDFTLTISSLQPEDXATYYCQQYYSGXTFGGGTKVEIK

SEQ ID NO: 167 - protein, 17 aa, Artificial Sequence
  CDR2 Consensus
  MISC_FEATURE (7)..(7): X is D, R, L, K, F, S, Y, or V
  MISC_FEATURE (8)..(8): X is G, R, N, Q, E, L, K, S, Y, or V
  MISC_FEATURE (12)..(12): X is N, A, or S
  MISC_FEATURE (13)..(13): X is E or Q
  MISC_FEATURE (16)..(16): X is K or Q
YIDPYNXXAKYXXKFXG

SEQ ID NO: 168 - protein, 7 aa, Artificial Sequence
  CDR2 consensus
  misc_feature (1)..(1): X is N, A, V, W, S, T, R, H, G, I, or V
  misc_feature (2)..(2): X is A, N, I, L, T, or V
XXKTLAE

SEQ ID NO: 169 - protein, 119 aa, Artificial Sequence
  VH parental
  MISC_FEATURE (5)..(5): X is Q or V
  MISC_FEATURE (9)..(9): X is P or A
  MISC_FEATURE (11)..(11): X is V or L
  MISC_FEATURE (12)..(12): X is V or K
  MISC_FEATURE (16)..(16): X is S or A
  MISC_FEATURE (20)..(20): X is M or V
  MISC_FEATURE (38)..(38): X is K or R
  MISC_FEATURE (40)..(40): X is K or A
  MISC_FEATURE (44)..(44): X is G or R
  MISC_FEATURE (56)..(56): X is D, R, L, K, F, S, Y or V
  MISC_FEATURE (57)..(57): X is G, R, N, Q, E, L, K, S, Y, or V
  MISC_FEATURE (61)..(61): X is N, A, or S
  MISC_FEATURE (62)..(62): X is E or Q
  MISC_FEATURE (65)..(65): X is K or Q
  MISC_FEATURE (67)..(67): X is R or K
  MISC_FEATURE (68)..(68): X is A or V
  MISC_FEATURE (72)..(72): X is S or R
  MISC_FEATURE (74)..(74): X is K or T
  MISC_FEATURE (76)..(76): X is S, I, A, or T
  MISC_FEATURE (79)..(79): X is A or V
  MISC_FEATURE (85)..(85): X is R or S
  MISC_FEATURE (87)..(87): X is T or R
  MISC_FEATURE (89)..(89): X is D or E
  MISC_FEATURE (91)..(91): X is S or T
  MISC_FEATURE (92)..(92): X is A or V
  MISC_FEATURE (104)..(104): X is W, A, D, E, F, G, I, K, N, Q, R, S, T, V, or Y
  MISC_FEATURE (111)..(111): X is A or Q
EVQLXQSGXEXXKPGXSVKXSCKASGYTFSSYVMHWVXQXPGQXLEWIGYIDPYNXXAKYXXKFXGXXTLTXDXSXSTXYMELSXLXSXDXXVYYCARGGPYGXYFDVWGXGTTVTVSS

SEQ ID NO: 170 - protein, 119 aa, Artificial Sequence
  Variable heavy consensus
  MISC_FEATURE (16)..(16): X is A or S
  MISC_FEATURE (44)..(44): X is R or G
  MISC_FEATURE (56)..(56): X is D, R, L, K, F, S, Y, or V
  MISC_FEATURE (57)..(57): X is G, R, N, Q, E, L, K, S, Y or V
  MISC_FEATURE (61)..(61): X is N, A, or S
  MISC_FEATURE (62)..(62): X is E or Q
  MISC_FEATURE (65)..(65): X is K or Q
  MISC_FEATURE (72)..(72): X is R or S
  MISC_FEATURE (74)..(74): X is T or K
  MISC_FEATURE (76)..(76): X is A, T, or I
  MISC_FEATURE (79)..(79): X is A or V
  MISC_FEATURE (85)..(85): X is S or R
  MISC_FEATURE (89)..(89): X is E or D
  MISC_FEATURE (92)..(92): X is A or V
  MISC_FEATURE (104)..(104): X is W, A, D, E, F, G, I, K, N, Q, R, S, T, V, or Y
EVQLVQSGAEVKKPGXSVKVSCKASGYTFSSYVMHWVRQAPGQXLEWIGYIDPYNXXAKYXXKFXGRVTLTXDXSXSTXYMELSXLRSXDTXVYYCARGGPYGXYFDVWGQGTTVTVSS

SEQ ID NO: 171 - protein, 107 aa, Artificial Sequence
  variable light consensus
  MISC_FEATURE (9)..(9): X is A or S
  MISC_FEATURE (17)..(17): X is E or D
  MISC_FEATURE (18)..(18): X is T or R
  MISC_FEATURE (40)..(40): X is Q or P
  MISC_FEATURE (43)..(43): X is S, A, or V
  MISC_FEATURE (45)..(45): X is Q or K
  MISC_FEATURE (48)..(48): X is V or I
  MISC_FEATURE (50)..(50): X is N, A, Y, W, S, T, I, or V
  MISC_FEATURE (51)..(51): X is A, N, I, L, T, or V
  MISC_FEATURE (70)..(70): X is Q or D
  MISC_FEATURE (72)..(72): X is S or T
  MISC_FEATURE (74)..(74): X is K or T
  MISC_FEATURE (76)..(76): X is N or S
  MISC_FEATURE (83)..(83): X is F or V
  MISC_FEATURE (84)..(84): X is G or A
  MISC_FEATURE (100)..(100): X is A or Q
  MISC_FEATURE (103)..(103): X is T or R
  MISC_FEATURE (106)..(106): X is L or I
DIQMTQSPXSLSASVGXXVTITCRASEHIYSYLSWYQQKXGKXPXLLXYXXKTLAEGVPSRFSGSGSGTXFXLXIXSLQPEDXXTYYCQHHFGSPLTFGXGTXLEXK

SEQ ID NO: 172 - protein, 107 aa, Artificial Sequence
  variable light consensus
  MISC_FEATURE (43)..(43): X is A or V
  MISC_FEATURE (50)..(50): X is N, A, Y, W, S, T, I, or V
  MISC_FEATURE (51)..(51): X is A, N, I, L, T, or V
  MISC_FEATURE (70)..(70): X is D or Q
  MISC_FEATURE (83)..(83): X is F or V
DIQMTQSPSSLSASVGDRVTITCRASEHIYSYLSWYQQKPGKXPKLLIYXXKTLAEGVPSRFSGSGSGTXFTLTISSLQPEDXATYYCQHHFGSPLTFGQGTRLEIK

SEQ ID NO: 173 - protein, 10 aa, Artificial Sequence
  CDR3 consensus
  MISC_FEATURE (6)..(6): X is W, A, D, E, F, G, I, K, N, Q, R, S, T, V, or Y
GGPYGXYFDV

SEQ ID NO: 174 - protein, 10 aa, Artificial Sequence
  CDR3
GGPYGAYFDV

SEQ ID NO: 175 - protein, 10 aa, Artificial Sequence
  CDR3
GGPYGDYFDV

SEQ ID NO: 176 - protein, 10 aa, Artificial Sequence
  CDR3
GGPYGEYFDV

SEQ ID NO: 177 - protein, 10 aa, Artificial Sequence
  CDR3
GGPYGFYFDV

SEQ ID NO: 178 - protein, 10 aa, Artificial Sequence
  CDR3
GGPYGGYFDV

SEQ ID NO: 179 - protein, 10 aa, Artificial Sequence
  CDR3
GGPYGIYFDV

SEQ ID NO: 180 - protein, 10 aa, Artificial Sequence
  CDR3
GGPYGKYFDV

SEQ ID NO: 181 - protein, 10 aa, Artificial Sequence
  CDR3
GGPYGNYFDV

SEQ ID NO: 182 - protein, 10 aa, Artificial Sequence
  CDR3
GGPYGQYFDV

SEQ ID NO: 183 - protein, 10 aa, Artificial Sequence
  CDR3
GGPYGRYFDV

SEQ ID NO: 184 - protein, 10 aa, Artificial Sequence
  CDR3
GGPYGSYFDV

SEQ ID NO: 185 - protein, 10 aa, Artificial Sequence
  CDR3
GGPYGTYFDV

SEQ ID NO: 186 - protein, 15 aa, Artificial Sequence
  CDR3
GGPYGVYFDVSEQID

SEQ ID NO: 187 - protein, 10 aa, Artificial Sequence
  CDR3
GGPYGYYFDV

SEQ ID NO: 188 - protein, 5 aa, Artificial Sequence
  CDRH1
GAWMD

SEQ ID NO: 189 - protein, 19 aa, Artificial Sequence
  CDRH2
EIRTKVNNHATNYGESVKG

SEQ ID NO: 190 - protein, 10 aa, Artificial Sequence
  CDRH3
ALYDGFYFDY

SEQ ID NO: 191 - protein, 12 aa, Artificial Sequence
  CDRL1
SASSSVSSGYLY

SEQ ID NO: 192 - protein, 7 aa, Artificial Sequence
  CDRL2
GTSTLAS

SEQ ID NO: 193 - protein, 9 aa, Artificial Sequence
  CDRL3
HQWSSFPYT

SEQ ID NO: 194 - protein, 121 aa, Artificial Sequence
  variable heavy
EVKLEESGGGLVQPGGSMKLSCVASGFTFSGAWMDWVRQSPEKGLEWVAEIRTKVNNHATNYGESVKGRFTISRDDSKSSVYLQMNNLRAEDSGIYYCRGALYDGFYFDYWGQGTTLTVSS

SEQ ID NO: 195 - protein, 108 aa, Artificial Sequence
  variable light
QIVLTQSPAIMSASPGEKVNLTCSASSSVSSGYLYWYQQKPGSSPKLWIYGTSTLASGVPARFSGSGSGTSYSLTISNMEAEDAASYFCHQWSSFPYTFGGGTKLEMK

SEQ ID NO: 196 - protein, 121 aa, Artificial Sequence
  variable heavy consensus
  MISC_FEATURE (33)..(33): X is W, A, R, N, D, Q, E, G, H, I, L, K, F, P, S, T, Y or V
  MISC_FEATURE (34)..(34): X is M, V, L, I, G, A, S, T
  MISC_FEATURE (76)..(76): X is D, A, R, N, Q, E, G, H, I, L, K, F, S, T, Y, or V
  MISC_FEATURE (79)..(79): X is S or N
  MISC_FEATURE (80)..(80): X is T or S
  MISC_FEATURE (85)..(85): X is M or L
  MISC_FEATURE (86)..(86): X is N or S
  MISC_FEATURE (87)..(87): X is S or N
  MISC_FEATURE (93)..(93): X is T or S
  MISC_FEATURE (104)..(104): X is D, A, R, N, Q, E, G, H, I, L, K, F, P, S, T, W, Y, or V
  MISC_FEATURE (105)..(105): X is G, A, R, N, D, Q, E, H, I, L, K, F, P, S, T, W, Y or V
EVQLVESGGGLVQPGGSLKLSCAASGFTFSGAXXDWVRQAPGKGLEWVAEIRTKVNNHATNYGESVKGRFTISRDXSKXXVYLQXXXLRAEDXAVYYCRGALYXXFYFDYWGQGTLVTVSS

SEQ ID NO: 197 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDGFYFDYWGQGTLVTVSS

SEQ ID NO: 198 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDGFYFDYWGQGTLVTVSS

SEQ ID NO: 199 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDGFYFDYWGQGTLVTVSS

SEQ ID NO: 200 - protein, 108 aa, Artificial Sequence
  variable light consensus
  MISC_FEATURE (20)..(20): X is T or S
  MISC_FEATURE (46)..(46): X is K or R
  MISC_FEATURE (48)..(48): X is W, A, R, N, D, Q, E, G, H, I, L, K, F, P, S, T, Y, or V
  MISC_FEATURE (59)..(59): X is V or I
  MISC_FEATURE (79)..(79): X is L, V, or I
  MISC_FEATURE (84)..(84): X is F, V, L, I, or T
  MISC_FEATURE (92)..(92): X is W, A, R, N, D, Q, E, G, H, I, L, K, F, P, S, T, Y or V
  MISC_FEATURE (107)..(107): X is I or L
EIVLTQSPATLSLSPGERAXLSCSASSSVSSGYLYWYQQKPGQAPXLXIYGTSTLASGXPARFSGSGSGTDYTLTISSXEPEDXAVYYCHQXSSFPYTFGQGTKLEXK

SEQ ID NO: 201 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQWSSFPYTFGQGTKLEIK

SEQ ID NO: 202 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQWSSFPYTFGQGTKLEIK

SEQ ID NO: 203 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQWSSFPYTFGQGTKLEIK

SEQ ID NO: 204 - protein, 5 aa, Artificial Sequence
  CDRH1
EFTMH

SEQ ID NO: 205 - protein, 17 aa, Artificial Sequence
  CDRH2
GLKPDNGGISYNQKFKG

SEQ ID NO: 206 - protein, 10 aa, Artificial Sequence
  CDRH3
GAYYRYDADY

SEQ ID NO: 207 - protein, 11 aa, Artificial Sequence
  CDRL1
KASQDVKTAVA

SEQ ID NO: 208 - protein, 7 aa, Artificial Sequence
  CDRL2
SASYRNT

SEQ ID NO: 209 - protein, 9 aa, Artificial Sequence
  CDRL3
QQHYSTPFT

SEQ ID NO: 210 - protein, 119 aa, Artificial Sequence
  variable heavy parental
EVQLQQSGPELVKPGASVKISCKTSGYTFTEFTMHWVKQSHGKSLEWIGGLKPDNGGISYNQKFKGRATLAVDKSSNTAYMELRSLTSEDSAVYYCARGAYYRYDADYWGQGTTLTVSS

SEQ ID NO: 211 - protein, 107 aa, Artificial Sequence
  variable light parental
DIVLTQSHKFMSTSVGDRVSITCKASQDVKTAVAWYQQKSGQSPKLLIYSASYRNTGVPDRFTGSGSGTDFTFTIDSVQAEDLAVYFCQQHYSTPFTFGTGTKLELK

SEQ ID NO: 212 - protein, 119 aa, Artificial Sequence
  variable heavy consensus
  misc_feature (24)..(24): X is T or V
  misc_feature (34)..(34): X is M, V, L, I, G, A, S, or T
  misc_feature (38)..(38): X is K or R
  MISC_FEATURE (55)..(55): X is N, A, R, D, Q, E, G, H, I, L, K, F, P, S, T, W, Y, or Y
  MISC_FEATURE (56)..(56): X is G, A, R, N, D, W, E, H, I, L, K, F, P, S, T, W, Y or V
  misc_feature (74)..(74): X is K, T, D, or S
  misc_feature (77)..(77): X is N or S
  misc_feature (81)..(81): X is M, V, L, I, G, A, S, or T
  misc_feature (91)..(91): X is T or S
  misc_feature (105)..(105): X is D, A, R, N, Q, E, G, H, I, L, K, F, P, S, T, W, Y or V
  misc_feature (106)..(106): X is A, R, N, D, Q, E, G, H, I, L, K, F, P, S, T, W, Y, V, or M
EVQLVQSGAEVKKPGASVKISCKXSGYTFTEFTXHWVXQAPGKGLEWIGGLKPDXXGISYNQKFKGRATLTVDXSTXTAYXELSSLRSEDXAVYYCARGAYYRYXXDYWGQGTLVTVSS

SEQ ID NO: 213 - protein, 119 aa, Artificial Sequence
  variable heavy
EVQLVQSGAEVKKPGASVKISCKTSGYTFTEFTMHWVKQAPGKGLEWIGGLKPDNGGISYNQKFKGRATLTVDKSTNTAYMELSSLRSEDTAVYYCARGAYYRYDADYWGQGTLVTVSS

SEQ ID NO: 214 - protein, 119 aa, Artificial Sequence
  variable heavy
EVQLVQSGAEVKKPGASVKISCKTSGYTFTEFTMHWVRQAPGKGLEWIGGLKPDNGGISYNQKFKGRATLTVDKSTSTAYMELSSLRSEDTAVYYCARGAYYRYDADYWGQGTLVTVSS

SEQ ID NO: 215 - protein, 119 aa, Artificial Sequence
  variable heavy
EVQLVQSGAEVKKPGASVKISCKVSGYTFTEFTMHWVRQAPGKGLEWIGGLKPDNGGISYNQKFKGRATLTVDTSTSTAYMELSSLRSEDTAVYYCARGAYYRYDADYWGQGTLVTVSS

SEQ ID NO: 216 - protein, 107 aa, Artificial Sequence
  variable light consensus
  MISC_FEATURE (55)..(55): X is N, Q, D, or E
  MISC_FEATURE (56)..(56): X is T, S, or A
  MISC_FEATURE (60)..(60): X is D or S
  MISC_FEATURE (73)..(73): X is F or L
DIQLTQSPSSLSASVGDRVTITCKASQDVKTAVAWYQQKPGKAPKLLIYSASYRXXGVPXRFSGSGSGTDFTXTISSLQPEDFATYYCQQHYSTPFTFGQGTKLEIK

SEQ ID NO: 217 - protein, 107 aa, Artificial Sequence
  variable light
DIQLTQSPSSLSASVGDRVTITCKASQDVKTAVAWYQQKPGKAPKLLIYSASYRNTGVPDRFSGSGSGTDFTFTISSLQPEDFATYYCQQHYSTPFTFGQGTKLEIK

SEQ ID NO: 218 - protein, 107 aa, Artificial Sequence
  variable light
DIQLTQSPSSLSASVGDRVTITCKASQDVKTAVAWYQQKPGKAPKLLIYSASYRNTGVPSRFSGSGSGTDFTFTISSLQPEDFATYYCQQHYSTPFTFGQGTKLEIK

SEQ ID NO: 219 - protein, 107 aa, Artificial Sequence
  variable light
DIQLTQSPSSLSASVGDRVTITCKASQDVKTAVAWYQQKPGKAPKLLIYSASYRNTGVPSRFSGSGSGTDFTLTISSLQPEDFATYYCQQHYSTPFTFGQGTKLEIK

SEQ ID NO: 220 - protein, 10 aa, Artificial Sequence
  CDRH3
ALYEGFYFDY

SEQ ID NO: 221 - protein, 10 aa, Artificial Sequence
  CDRH3
ALYDAFYFDY

SEQ ID NO: 222 - protein, 10 aa, Artificial Sequence
  CDRH3
ALYDSFYFDY

SEQ ID NO: 223 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYEGFYFDYWGQGTLVTVSS

SEQ ID NO: 224 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDAFYFDYWGQGTLVTVSS

SEQ ID NO: 225 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDSFYFDYWGQGTLVTVSS

SEQ ID NO: 226 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDEFYFDYWGQGTLVTVSS

SEQ ID NO: 227 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDAFYFDYWGQGTLVTVSS

SEQ ID NO: 228 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDSFYFDYWGQGTLVTVSS

SEQ ID NO: 229 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYEGFYFDYWGQGTLVTVSS

SEQ ID NO: 230 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDAFYFDYWGQGTLVTVSS

SEQ ID NO: 231 - protein, 121 aa, Artificial Sequence
  variable heavy
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDSFYFDYWGQGTLVTVSS

SEQ ID NO: 232 - protein, 9 aa, Artificial Sequence
  CDRL3
HQASSFPYT

SEQ ID NO: 233 - protein, 9 aa, Artificial Sequence
  CDRL3
HQDSSFPYT

SEQ ID NO: 234 - protein, 9 aa, Artificial Sequence
  CDRL3
HQESSFPYT

SEQ ID NO: 235 - protein, 9 aa, Artificial Sequence
  CDRL3
HQFSSFPYT

SEQ ID NO: 236 - protein, 9 aa, Artificial Sequence
  CDRL3
HQGSSFPYT

SEQ ID NO: 237 - protein, 9 aa, Artificial Sequence
  CDRL3
HQHSSFPYT

SEQ ID NO: 238 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQASSFPYTFGQGTKLEIK

SEQ ID NO: 239 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQDSSFPYTFGQGTKLEIK

SEQ ID NO: 240 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQESSFPYTFGQGTKLEIK

SEQ ID NO: 241 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQFSSFPYTFGQGTKLEIK

SEQ ID NO: 242 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQGSSFPYTFGQGTKLEIK

SEQ ID NO: 243 - protein, 108 aa, Artificial Sequence
  variable light
EIVLTQSPATLSLSPGERATLSCSASSSVSSGYLYWYQQKPGQAPKLWIYGTSTLASGVPARFSGSGSGTDYTLTISSLEPEDFAVYYCHQHSSFPYTFGQGTKLEIK

SEQ ID NO: 244 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQASSFPYTFGQGTKLEIK

SEQ ID NO: 245 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQDSSFPYTFGQGTKLEIK

SEQ ID NO: 246 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQESSFPYTFGQGTKLEIK

SEQ ID NO: 247 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQFSSFPYTFGQGTKLEIK

SEQ ID NO: 248 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQGSSFPYTFGQGTKLEIK

SEQ ID NO: 249 - protein, 108 aa, Artificial Sequence
  variable light
EIVLTQSPATLSLSPGERATLSCSASSSVSSGYLYWYQQKPGQAPRLWIYGTSTLASGVPARFSGSGSGTDYTLTISSLEPEDFAVYYCHQHSSFPYTFGQGTKLEIK

SEQ ID NO: 250 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQASSFPYTFGQGTKLEIK

SEQ ID NO: 251 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQDSSFPYTFGQGTKLEIK

SEQ ID NO: 252 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQESSFPYTFGQGTKLEIK

SEQ ID NO: 253 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQFSSFPYTFGQGTKLEIK

SEQ ID NO: 254 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQGSSFPYTFGQGTKLEIK

SEQ ID NO: 255 - protein, 108 aa, Artificial Sequence
  variable light
EIVLTQSPATLSLSPGERATLSCSASSSVSSGYLYWYQQKPGQAPRLWIYGTSTLASGIPARFSGSGSGTDYTLTISSLEPEDFAVYYCHQHSSFPYTFGQGTKLEIK

SEQ ID NO: 256 - protein, 17 aa, Artificial Sequence
  CDRH2
GLKPDQGGISYNQKFKG

SEQ ID NO: 257 - protein, 17 aa, Artificial Sequence
  CDRH2
GLKPDDGGISYNQKFKG

SEQ ID NO: 258 - protein, 17 aa, Artificial Sequence
  CDRH2
GLKPDNAGISYNQKFKG

SEQ ID NO: 259 - protein, 17 aa, Artificial Sequence
  CDRH2
GLKPDTGGISYNQKFKG

SEQ ID NO: 260 - protein, 17 aa, Artificial Sequence
  CDRH2
GLKPDSGGISYNQKFKG

SEQ ID NO: 261 - protein, 17 aa, Artificial Sequence
  CDRH2
GLKPDGGGISYNQKFKG

SEQ ID NO: 262 - protein, 17 aa, Artificial Sequence
  CDRH2
GLKPDNSGISYNQKFKG

SEQ ID NO: 263 - protein, 17 aa, Artificial Sequence
  CDRH2
GLKPDNTGISYNQKFKG

SEQ ID NO: 264 - protein, 119 aa, Artificial Sequence
  variable heavy
EVQLVQSGAEVKKPGASVKISCKTSGYTFTEFTMHWVKQAPGKGLEWIGGLKPDQGGISYNQKFKGRATLTVDKSTNTAYMELSSLRSEDTAVYYCARGAYYRYDADYWGQGTLVTVSS

SEQ ID NO: 265 - protein, 119 aa, Artificial Sequence
  variable heavy
EVQLVQSGAEVKKPGASVKISCKTSGYTFTEFTMHWVKQAPGKGLEWIGGLKPDDGGISYNQKFKGRATLTVDKSTNTAYMELSSLRSEDTAVYYCARGAYYRYDADYWGQGTLVTVSS

SEQ ID NO: 266 - protein, 119 aa, Artificial Sequence
  variable heavy
EVQLVQSGAEVKKPGASVKISCKTSGYTFTEFTMHWVKQAPGKGLEWIGGLKPDNAGISYNQKFKGRATLTVDKSTNTAYMELSSLRSEDTAVYYCARGAYYRYDADYWGQGTLVTVSS

SEQ ID NO: 267 - protein, 119 aa, Artificial Sequence
  variable heavy
EVQLVQSGAEVKKPGASVKISCKTSGYTFTEFTMHWVKQAPGKGLEWIGGLKPDTGGISYNQKFKGRATLTVDKSTNTAYMELSSLRSEDTAVYYCARGAYYRYDADYWGQGTLVTVSS

SEQ ID NO: 268 - protein, 119 aa, Artificial Sequence
  variable heavy
EVQLVQSGAEVKKPGASVKISCKTSGYTFTEFTMHWVKQAPGKGLEWIGGLKPDSGGISYNQKFKGRATLTVDKSTNTAYMELSSLRSEDTAVYYCARGAYYRYDADYWGQGTLVTVSS

SEQ ID NO: 269 - protein, 119 aa, Artificial Sequence
  variable heavy
EVQLVQSGAEVKKPGASVKISCKTSGYTFTEFTMHWVKQAPGKGLEWIGGLKPDGGGISYNQKFKGRATLTVDKSTNTAYMELSSLRSEDTAVYYCARGAYYRYDADYWGQGTLVTVSS

SEQ ID NO: 270 - protein, 119 aa, Artificial Sequence
  variable heavy
EVQLVQSGAEVKKPGASVKISCKTSGYTFTEFTMHWVKQAPGKGLEWIGGLKPDNSGISYNQKFKGRATLTVDKSTNTAYMELSSLRSEDTAVYYCARGAYYRYDADYWGQGTLVTVSS

SEQ ID NO: 271 - protein, 119 aa, Artificial Sequence
  variable heavy
EVQLVQSGAEVKKPGASVKISCKTSGYTFTEFTMHWVKQAPGKGLEWIGGLKPDNTGISYNQKFKGRATLTVDKSTNTAYMELSSLRSEDTAVYYCARGAYYRYDADYWGQGTLVTVSS

SEQ ID NO: 272 - protein, 119 aa, Artificial Sequence
  variable heavy
EVQLVQSGAEVKKPGASVKISCKTSGYTFTEFTMHWVRQAPGKGLEWIGGLKPDQGGISYNQKFKGRATLTVDKSTSTAYMELSSLRSEDTAVYYCARGAYYRYDADYWGQGTLVTVSS

SEQ ID NO: 273 - protein, 119 aa, Artificial Sequence
  variable heavy
EVQLVQSGAEVKKPGASVKISCKTSGYTFTEFTMHWVRQAPGKGLEWIGGLKPDDGGISYNQKFKGRATLTVDKSTSTAYMELSSLRSEDTAVYYCARGAYYRYDADYWGQGTLVTVSS

SEQ ID NO: 274 - protein, 119 aa, Artificial Sequence
  variable heavy
EVQLVQSGAEVKKPGASVKISCKTSGYTFTEFTMHWVRQAPGKGLEWIGGLKPDNAGISYNQKFKGRATLTVDKSTSTAYMELSSLRSEDTAVYYCARGAYYRYDADYWGQGTLVTVSS

SEQ ID NO: 275 - protein, 119 aa, Artificial Sequence
  variable heavy
EVQLVQSGAEVKKPGASVKISCKTSGYTFTEFTMHWVRQAPGKGLEWIGGLKPDTGGISYNQKFKGRATLTVDKSTSTAYMELSSLRSEDTAVYYCARGAYYRYDADYWGQGTLVTVSS

SEQ ID NO: 276 - protein, 119 aa, Artificial Sequence
  variable heavy
EVQLVQSGAEVKKPGASVKISCKTSGYTFTEFTMHWVRQAPGKGLEWIGGLKPDSGGISYNQKFKGRATLTVDKSTSTAYMELSSLRSEDTAVYYCARGAYYRYDADYWGQGTLVTVSS

SEQ ID NO: 277 - protein, 119 aa, Artificial Sequence
  variable heavy
EVQLVQSGAEVKKPGASVKISCKTSGYTFTEFTMHWVRQAPGKGLEWIGGLKPDGGGISYNQKFKGRATLTVDKSTSTAYMELSSLRSEDTAVYYCARGAYYRYDADYWGQGTLVTVSS

SEQ ID NO: 278 - protein, 119 aa, Artificial Sequence
  variable heavy
EVQLVQSGAEVKKPGASVKISCKTSGYTFTEFTMHWVRQAPGKGLEWIGGLKPDNSGISYNQKFKGRATLTVDKSTSTAYMELSSLRSEDTAVYYCARGAYYRYDADYWGQGTLVTVSS

SEQ ID NO: 279 - protein, 119 aa, Artificial Sequence
  variable heavy
EVQLVQSGAEVKKPGASVKISCKTSGYTFTEFTMHWVRQAPGKGLEWIGGLKPDNTGISYNQKFKGRATLTVDKSTSTAYMELSSLRSEDTAVYYCARGAYYRYDADYWGQGTLVTVSS

SEQ ID NO: 280 - protein, 119 aa, Artificial Sequence
  variable heavy
EVQLVQSGAEVKKPGASVKISCKVSGYTFTEFTMHWVRQAPGKGLEWIGGLKPDQGGISYNQKFKGRATLTVDTSTSTAYMELSSLRSEDTAVYYCARGAYYRYDADYWGQGTLVTVSS

SEQ ID NO: 281 - protein, 119 aa, Artificial Sequence
  variable heavy
EVQLVQSGAEVKKPGASVKISCKVSGYTFTEFTMHWVRQAPGKGLEWIGGLKPDDGGISYNQKFKGRATLTVDTSTSTAYMELSSLRSEDTAVYYCARGAYYRYDADYWGQGTLVTVSS

SEQ ID NO: 282 - protein, 119 aa, Artificial Sequence
  variable heavy
EVQLVQSGAEVKKPGASVKISCKVSGYTFTEFTMHWVRQAPGKGLEWIGGLKPDNAGISYNQKFKGRATLTVDTSTSTAYMELSSLRSEDTAVYYCARGAYYRYDADYWGQGTLVTVSS

SEQ ID NO: 283 - protein, 119 aa, Artificial Sequence
  variable heavy
EVQLVQSGAEVKKPGASVKISCKVSGYTFTEFTMHWVRQAPGKGLEWIGGLKPDTGGISYNQKFKGRATLTVDTSTSTAYMELSSLRSEDTAVYYCARGAYYRYDADYWGQGTLVTVSS

SEQ ID NO: 284 - protein, 119 aa, Artificial Sequence
  variable heavy
EVQLVQSGAEVKKPGASVKISCKVSGYTFTEFTMHWVRQAPGKGLEWIGGLKPDSGGISYNQKFKGRATLTVDTSTSTAYMELSSLRSEDTAVYYCARGAYYRYDADYWGQGTLVTVSS

SEQ ID NO: 285 - protein, 119 aa, Artificial Sequence
  variable heavy
EVQLVQSGAEVKKPGASVKISCKVSGYTFTEFTMHWVRQAPGKGLEWIGGLKPDGGGISYNQKFKGRATLTVDTSTSTAYMELSSLRSEDTAVYYCARGAYYRYDADYWGQGTLVTVSS

SEQ ID NO: 286 - protein, 119 aa, Artificial Sequence
  variable heavy
EVQLVQSGAEVKKPGASVKISCKVSGYTFTEFTMHWVRQAPGKGLEWIGGLKPDNSGISYNQKFKGRATLTVDTSTSTAYMELSSLRSEDTAVYYCARGAYYRYDADYWGQGTLVTVSS

SEQ ID NO: 287 - protein, 119 aa, Artificial Sequence
  variable heavy
EVQLVQSGAEVKKPGASVKISCKVSGYTFTEFTMHWVRQAPGKGLEWIGGLKPDNTGISYNQKFKGRATLTVDTSTSTAYMELSSLRSEDTAVYYCARGAYYRYDADYWGQGTLVTVSS

SEQ ID NO: 288 - protein, 15 aa, Artificial Sequence
  hTIGIT epitope 24-41
SSTTAQVNWEQQDQL

SEQ ID NO: 289 - protein, 9 aa, Artificial Sequence
  hTIGIT epitope 85-93
IYHTYPDGT

SEQ ID NO: 290 - protein, 5 aa, Artificial Sequence
  hTIGIT epitope 96-100
GRIFL

SEQ ID NO: 291 - protein, 330 aa, Artificial Sequence [sequence omitted]

SEQ ID NO: 292 - protein, 325 aa, Artificial Sequence
  Heavy chain constant domain IgG4 S228P
TKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 105 aa, Artificial Sequence [sequence omitted]

(19) United States
(12) Patent Application Publication
(10) Pub. No.: US 2020/0354453 A1
(43) Pub. Date: Nov. 12, 2020

(54) STABLE FORMULATIONS OF ANTI-TIGIT ANTIBODIES ALONE AND IN COMBINATION WITH PROGRAMMED DEATH RECEPTOR 1 (PD-1) ANTIBODIES AND METHODS OF USE THEREOF

(71) Applicant: Merck Sharp & Dohme Corp., Rahway, NJ (US)

(72) Inventors: Arnab De, Edison, NJ (US); Chakravarthy Nachu Narasimhan, Scotch Plains, NJ (US)

(73) Assignee: Merck Sharp & Dohme Corp., Rahway, NJ (US)

(21) Appl. No.: 16/610,188

(22) PCT Filed: May 1, 2018
(86) PCT No.: PCT/US2018/030516

§ 371 Date: Nov. 1, 2019

Related U.S. Application Data
(60) Provisional application No. 62/500,278, filed on May 2, 2017.

(51) Int. Cl. C07K 16/28; A61K 47/54

(52) U.S. Cl. CPC C07K 16/2818; A61K 2039/505; A61K 47/547; C07K 16/2809

(57) ABSTRACT

The present invention relates to stable formulations of antibodies against T cell immunoreceptor with Ig and ITIM domains (TIGIT), optionally further containing an anti-human programmed death receptor 1 (PD-1) antibody or antigen binding fragment thereof. Also provided are methods of treating various cancers and chronic infections with the formulations of the invention.

### CROSS-REFERENCE TO RELATED APPLICATIONS

This application claims the benefit of U.S. Ser. No. 62/500,278, filed May 2, 2017, the contents of which are hereby incorporated by reference in their entirety.

### FIELD OF THE INVENTION

The invention relates to formulations of therapeutic antibodies and their use in treating various disorders. In one aspect, the invention relates to formulations comprising antibodies or antigen binding fragments thereof that bind to T cell immunoreceptor with Ig and ITIM domains (TIGIT). In another aspect, such formulation further comprises an anti-human programmed death receptor 1 (PD-1) antibody or antigen binding fragment thereof. Also provided are methods of treating various cancers and chronic infections with the formulations of the invention.

### REFERENCE TO SEQUENCE LISTING SUBMITTED ELECTRONICALLY

The sequence listing of the present application is submitted electronically via EFS-Web as an ASCII formatted sequence listing with a file name “24453WOPCT-SEQTXT-01MAY2018.TXT”, creation date of May 1, 2018, and a size of 227 Kb. This sequence listing submitted via EFS-Web is part of the specification and is herein incorporated by reference in its entirety.

### BACKGROUND OF THE INVENTION

Antibody drugs for use in humans may differ somewhat in the amino acid sequence of their constant domains, or in their framework sequences within the variable domains, but they typically differ most dramatically in the CDR sequences. Even antibodies binding to the same protein, the same polypeptide, or even potentially the same epitope may comprise entirely different CDR sequences. Therapeutic antibodies for use in human beings can also be obtained from human germline antibody sequence or from non-human (e.g. rodent) germline antibody sequences, such as in humanized antibodies, leading to yet further diversity in potential CDR sequences. These sequence differences result in different stabilities in solution and different responsiveness to solution parameters. In addition, small changes in the arrangement of amino acids or changes in one or a few amino acid residues can result in dramatically different antibody stability and susceptibility to sequence-specific degradation pathways. As a consequence, it is not possible at present to predict the solution conditions necessary to optimize antibody stability. Each antibody must be studied individually to determine the optimum solution formulation. Bhambhani et al. (2012) J. Pharm. Sci. 101:1120.

Antibodies are also relatively high molecular weight proteins (˜150,000 Da), for example as compared with other therapeutic proteins such as hormones and cytokines. As a consequence, it is frequently necessary to dose with relatively high weight amounts of antibody drugs to achieve the desired molar concentrations of drug. In addition, it is often desirable to administer antibody drugs subcutaneously, as this enables self-administration. Self-administration avoids the time and expense associated with visits to a medical facility for administration, e.g., intravenously. Subcutaneous delivery is limited by the volume of solution that can be practically delivered at an injection site in a single injection, which is generally about 1 to 1.5 ml. Subcutaneous self-administration is typically accomplished using a pre-filled syringe or autoinjector filled with a liquid solution formulation of the drug, rather than a lyophilized form, to avoid the need for the patient to re-suspend the drug prior to injection. Antibody drugs must be stable during storage to ensure efficacy and consistent dosing, so it is critical that whatever formulation is chosen supports desirable properties, such as high concentration, clarity and acceptable viscosity, and that also maintains these properties and drug efficacy over an acceptably long shelf-life under typical storage conditions.

TIGIT (T cell immunoreceptor with Ig and ITIM domains) is an immunomodulatory receptor expressed primarily on activated T cells and NK cells. TIGIT is also known as VSIG9; VSTM3; and WUCAM. Its structure shows one extracellular immunoglobulin domain, a type 1 transmembrane region and two ITIM motifs. TIGIT forms part of a co-stimulatory network that consists of positive (CD226) and negative (TIGIT) immunomodulatory receptors on T cells, and ligands expressed on APCs (CD155 and CD112).

An important feature in the structure of TIGIT is the presence of an immunoreceptor tyrosine-based inhibition motif (ITIM) in its cytoplasmic tail domain. As with PD-1 and TIGIT, the ITIM domain in the cytoplasmic region of TIGIT is predicted to recruit tyrosine phosphatases, such as SHP-1 and SHP-2, and subsequent de-phosphorylation of tyrosine residues with in the immunoreceptor tyrosine-base activation motifs (ITAM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity.

The need exists for stable formulations of anti-TIGIT antibodies for pharmaceutical use, e.g., for treating various cancers and infectious diseases, as well as for stable formulations of anti-TIGIT antibodies co-formulated with anti-human PD-1 antibodies. Preferably, such formulations will exhibit a long shelf-life, be stable when stored and transported, and will preferably exhibit stability over months to years under conditions typical for storage of drugs for self-administration, i.e. at refrigerator temperature in a syringe, resulting in a long shelf-life for the corresponding drug product.

### SUMMARY OF THE INVENTION

In one aspect, the invention includes a formulation of an anti-TIGIT antibody, or antigen binding fragment thereof, comprising (i) an anti-TIGIT antibody, or antigen binding fragment thereof; (ii) a buffer, (iii) a non-reducing sugar; (iv) a non-ionic surfactant; and (v) an antioxidant. In an embodiment, the formulation further comprises an anti-PD-1 antibody, e.g., pembrolizumab or nivolumab. In another embodiment, the formulation comprises a chelator.

In an embodiment of the invention, the formulation comprises (i) about 10 mg/ml to about 200 mg/ml of an anti-TIGIT antibody, or antigen binding fragment thereof; (ii) about 5 mM to about 20 mM buffer; (iii) about 6% to about 8% weight/volume (w/v) non-reducing sugar; (iv) about 0.01% to about 0.10% (w/v) non-ionic surfactant; and (v) about 1 mM to about 20 mM anti-oxidant. In an embodiment, the formulation further comprises an anti-PD-1 antibody, e.g., pembrolizumab or nivolumab. In another embodiment, the formulation further comprises a chelator. In one embodiment, the formulation has a pH between 4.5-6.5. In particular embodiments, the pH of the formulation is from about pH 5.5 to about pH 6.2. In a further embodiment, the pH of the formulation is from about pH 5.6 to about pH 6.0. In another embodiment, the pH of the formulation is about 5.7. In another embodiment, the pH of the formulation is about 5.8. In another embodiment, the pH of the formulation is about 5.9. In another embodiment, the pH of the formulation is about 6.0. In another embodiment, the pH of the formulation is about 6.1. In another embodiment, the pH of the formulation is about 6.2.

In one embodiment of the formulation, the buffer is L-histidine buffer or sodium acetate, the non-reducing sugar is sucrose, the non-ionic surfactant is polysorbate 80, and the anti-oxidant is methionine, or a pharmaceutically acceptable salt thereof. In one embodiment, the anti-oxidant is L-methionine. In another embodiment, the anti-oxidant is a pharmaceutically acceptable salt of L-methionine, such as, for example, methionine HCl.

In another embodiment, the formulation comprises (i) about 10 mg/ml to about 200 mg/ml of an anti-TIGIT antibody, or antigen binding fragment thereof; (ii) about 5 mM to about 20 mM of L-histidine buffer or about 5 mM to about 20 mM of sodium acetate buffer; (iii) about 6% to about 8% w/v sucrose; (iv) about 0.01% to about 0.10% (w/v) polysorbate 80; and (v) about 1 mM to about 20 mM L-methionine. In another embodiment, the formulation further comprises an anti-PD-1 antibody, e.g., pembrolizumab or nivolumab. In an embodiment, the formulation further comprises a chelator. In one embodiment, the chelator is present in an amount of about 1 μM to about 50 μM. In one embodiment, the chelator is DTPA. In another embodiment, the chelator is EDTA. In one embodiment, the buffer is L-histidine buffer. In one embodiment, the formulation comprises about 8 mM to about 12 mM of L-histidine buffer. In another embodiment, the formulation comprises about 5 mM to about 10 mM of L-methionine. In a further embodiment, the formulation comprises polysorbate 80 at a weight ratio of approximately 0.02% (w/v). In one embodiment, the anti-TIGIT formulation comprises sucrose at a weight ratio of about 7% (w/v). In any of these embodiments, the methionine is L-methionine.

In embodiments of the formulation, the concentration of the anti-TIGIT antibody or antigen binding fragment thereof is from about 10 mg/ml to about 100 mg/ml. In another embodiment, the concentration of the anti-TIGIT antibody or antigen binding fragment thereof is about 10 mg/ml, 12.5 mg/ml, 15 mg/ml, 20 mg/ml, 25 mg/ml, 50 mg/ml, 75 mg/ml or 100 mg/ml. In one embodiment, the concentration of the anti-TIGIT antibody or antigen binding fragment thereof is about 20 mg/ml. In one embodiment, the concentration of the anti-TIGIT antibody or antigen binding fragment thereof is about 25 mg/ml. In one embodiment, the concentration of the anti-TIGIT antibody or antigen binding fragment thereof is about 50 mg/ml. In one embodiment, the concentration of the anti-TIGIT antibody or antigen binding fragment thereof is about 75 mg/ml. In one embodiment, the concentration of the anti-TIGIT antibody or antigen binding fragment thereof is about 100 mg/ml.

In one aspect, provided is a formulation comprising about 20 mg/ml of an anti-TIGIT antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 25 mg/ml of an anti-TIGIT antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 50 mg/ml of an anti-TIGIT antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 75 mg/ml of an anti-TIGIT antibody or antigen binding fragment thereof, 10 mM L-histidine buffer, about 7% w/v sucrose, about 0.02% w/v polysorbate 80, and about 10 mM L-methionine.

In one aspect, provided is a formulation comprising about 100 mg/ml of an anti-TIGIT antibody or antigen binding fragment thereof, 10 mM L-histidine buffer, about 7% w/v sucrose, about 0.02% w/v polysorbate 80, and about 10 mM L-methionine.

In one aspect of any of the above formulation, the formulation has a pH of about 5.4 to about 6.2. In another aspect, the formulation has a pH of about 5.5-6.2. In another embodiment, the formulation has a pH of about 5.8-6.1. In another embodiment, the pH is about 5.8. In one embodiment, the pH is 5.9. In another embodiment the pH is 6.0. In a further embodiment the pH is 6.1.

In one aspect of any of the above formulations, the formulation comprises an anti-PD1 antibody or antigen binding fragment thereof. In one embodiment, the anti-PD1 antibody is pembrolizumab. In another aspect, the anti-PD1 antibody is nivolumab.

In another aspect, the formulation may further comprise a chelator. In one embodiment, the chelator is DTPA. In one embodiment, the chelator is EDTA. In one aspect, the chelator is present in an amount from about 1 μM to about 50 μM. In one embodiment, the formulation comprises about 5 μM of the chelator. In one embodiment, the formulation comprises about 10 μM of the chelator. In one embodiment, the formulation comprises about 15 μM of the chelator. In one embodiment, the formulation comprises about 20 μM of the chelator. In one embodiment, the formulation comprises about 25 μM of the chelator. In one embodiment, the formulation comprises about 30 μM of the chelator. In one embodiment, the formulation comprises about 35 μM of the chelator. In one embodiment, the formulation comprises about 40 μM of the chelator. In one embodiment, the formulation comprises about 45 μM of the chelator. In one embodiment, the formulation comprises about 50 μM of the chelator. In one embodiment, the chelating agent is DTPA, which is present at any of the amounts stated above. In another embodiment, the chelating agent is EDTA which is present at any of the amounts stated above.

In one embodiment, the formulation is contained in a glass vial. In another embodiment, the formulation is contained in an injection device. In another embodiment, the formulation is a liquid formulation. In one aspect, the formulation is froze to at least below −70° C. In another embodiment, the formulation is a reconstituted solution from a lyophilized formulation.

In certain embodiments, the formulation is stable at refrigerated temperature (2-8° C.) for at least 3 months, preferably 6 months, and more preferably 1 year, and even more preferably up to through 2 years. In one embodiment of the formulation, after 12 months at 5° C. the % monomer of the anti-TIGIT antibody is ≥90% as determined by size exclusion chromatography. In another embodiment of the formulation, after 12 months at 5° C. the % monomer of the anti-TIGIT antibody is ≥95% as determined by size exclusion chromatography. In another embodiment of the formulation, after 12 months at 5° C. the % heavy chain and light chain of the anti-TIGIT antibody is ≥90% as determined by reduced CE-SDS. In another embodiment of the formulation, after 12 months at 5° C. the % heavy chain and light chain of the anti-TIGIT antibody is ≥95% as determined by reduced CE-SDS. In another embodiment of the formulation, after 12 months at 5° C. the % intact IgG of the anti-TIGIT antibody is ≥90% as determined by non-reduced CE-SDS. In another embodiment of the formulation, after 12 months at 5° C. the % intact IgG of the anti-TIGIT antibody is ≥95% as determined by non-reduced CE-SDS.

In one aspect of any of the formulations described above, the formulation comprises an anti-TIGIT antibody or antigen-binding fragment thereof comprising three light chain CDRs and three heavy chain CDRs, wherein the light chain CDRs comprise CDRL1 of SEQ ID NO: 111 or variant thereof, CDRL2 of SEQ ID NO: 112 or variant thereof, CDRL3 of SEQ ID NO: 113 or variant thereof and the heavy chain CDRs comprise CDRH1 of SEQ ID NO: 108 or variant thereof, CDRH2 of SEQ ID NO: 154 or variant thereof, and CDHR3 of SEQ ID NO: 110 or variant thereof. In one aspect of any of the formulations described above, the formulation comprises an anti-TIGIT antibody or antigen-binding fragment thereof comprising three light chain CDRs and three heavy chain CDRs, wherein the light chain CDRs comprise CDRL1 of SEQ ID NO: 111, CDRL2 of SEQ ID NO: 112, CDRL3 of SEQ ID NO: 113 and the heavy chain CDRs comprise CDRH1 of SEQ ID NO: 108, CDRH2 of SEQ ID NO: 154, and CDHR3 of SEQ ID NO: 110. In another aspect, the formulation comprises an anti-TIGIT antibody or antigen binding fragment thereof comprising a heavy chain variable region comprising SEQ ID NO: 148 or variant thereof and a light chain variable region comprising SEQ ID NO: 152 or variant thereof. In another aspect, the formulation comprises an anti-TIGIT antibody or antigen binding fragment thereof comprising a heavy chain variable region comprising SEQ ID NO: 148 and a light chain variable region comprising SEQ ID NO: 152. In one aspect, the anti-TIGIT antibody or antigen binding fragment thereof further comprises a human heavy chain IgG1 constant domain comprising the amino acid sequence of SEQ ID NO:291 or variant thereof and a human kappa light chain constant domain comprising the amino acid sequence of SEQ ID NO:293 or variant thereof. In one aspect, the anti-TIGIT antibody or antigen binding fragment thereof further comprises a human heavy chain IgG1 constant domain comprising the amino acid sequence of SEQ ID NO:291 and a human kappa light chain constant domain comprising the amino acid sequence of SEQ ID NO:293. In another aspect, the anti-TIGIT antibody or antigen binding fragment thereof further comprises a human heavy chain IgG4 constant domain comprising the amino acid sequence of SEQ ID NO:292 and a human kappa light chain constant domain comprising the amino acid sequence of SEQ ID NO:293. In another aspect, the anti-TIGIT antibody or antigen binding fragment thereof further comprises a human heavy chain IgG4 constant domain comprising the amino acid sequence of SEQ ID NO:292 or variant thereof and a human kappa light chain constant domain comprising the amino acid sequence of SEQ ID NO:293 or variant thereof.

In one aspect, the invention provides a co-formulation of an anti-TIGIT antibody, or antigen binding fragment thereof and an anti-human PD-1 antibody, or antigen binding fragment thereof, comprising (i) an anti-TIGIT antibody, or antigen binding fragment thereof; (ii) an anti-human PD-1 antibody, or antigen binding fragment thereof, (ii) a buffer, (iii) a non-reducing sugar; (iv) a non-ionic surfactant; and (v) an antioxidant. In an embodiment, the co-formulation further comprises a chelator. In one embodiment the chelator is EDTA. In another embodiment, the chelator is DTPA. In one embodiment of the co-formulation, the ratio of the anti-human PD-1 antibody to the anti-TIGIT antibody is 1:2. In one embodiment of the co-formulation, the ratio of the anti-human PD-1 antibody to the anti-TIGIT antibody is 1:1. In one embodiment of the co-formulation, the ratio of the anti-human PD-1 antibody to the anti-TIGIT antibody is 2:1.

In an embodiment of the invention, the co-formulation comprises (i) about 1 mg/ml to about 200 mg/ml of an anti-TIGIT antibody, or antigen binding fragment thereof; (ii) about 1 mg/ml to about 200 mg/ml of an anti-human PD-1 antibody (iii) about 5 mM to about 20 mM buffer; (iv) about 6% to about 8% weight/volume (w/v) non-reducing sugar; (v) about 0.01% to about 0.10% (w/v) non-ionic surfactant; and (vi) about 1 mM to about 20 mM anti-oxidant.

In an embodiment, the co-formulation further comprises a chelator. In one embodiment, the chelator is present in an amount of about 1 μM to about 50 μM. In one embodiment, the chelator is DTPA. In another embodiment, the chelator is EDTA. In one embodiment of the co-formulation, the ratio of the anti-human PD-1 antibody to the anti-TIGIT antibody is 1:2. In one embodiment of the co-formulation, the ratio of the anti-human PD-1 antibody to the anti-TIGIT antibody is 1:1. In one embodiment of the co-formulation, the ratio of the anti-human PD-1 antibody to the anti-TIGIT antibody is 2:1. In one embodiment, the co-formulation has a pH between 4.5-6.5. In particular embodiments, the pH of the formulation is from about pH 5.5 to about pH 6.2. In a further embodiment, the pH of the formulation is from about pH 5.8-6.0.

In one embodiment of the co-formulation, the buffer is L-histidine buffer or sodium acetate buffer, the non-reducing sugar is sucrose, the non-ionic surfactant is polysorbate 80, and the anti-oxidant is L-methionine. In another embodiment, the co-formulation comprises (i) about 1 mg/ml to about 100 mg/ml of an anti-TIGIT antibody, or antigen binding fragment thereof; (ii) about 1 mg/ml to about 100 mg/ml of an anti-human PD-1 antibody or antigen binding fragment thereof; (iii) about 5 mM to about 20 mM of L-histidine or about 5 mM to about 20 mM of sodium acetate buffer; (iv) about 6% to about 8% w/v sucrose; (v) about 0.01% to about 0.10% (w/v) polysorbate 80; and (vi) about 1 mM to about 20 mM L-methionine. In an embodiment, the co-formulation optionally comprises a chelator. In one embodiment, the chelator is present in an amount of about 1 μM to about 50 μM. In one embodiment, the chelator is DTPA. In another embodiment, the chelator is EDTA. In one embodiment of the co-formulation, the buffer is L-histidine buffer. In one embodiment, the co-formulation comprises about 8 mM to about 12 mM of L-histidine buffer. In another embodiment, the co-formulation comprises about 5 mM to about 10 mM of L-methionine. In a further embodiment, the co-formulation comprises polysorbate 80 at a weight ratio of approximately 0.02% w/v. In one embodiment, co-formulation comprises sucrose at a weight ratio of about 7% (w/v).

In embodiments of the co-formulation, the concentration of the anti-TIGIT antibody or antigen binding fragment thereof is from about 1 mg/ml to about 100 mg/ml. In embodiments of the co-formulation, the concentration of the anti-TIGIT antibody or antigen binding fragment thereof is from about 10 mg/ml to about 100 mg/ml. In another embodiment, the concentration of the anti-TIGIT antibody or antigen binding fragment thereof is about 10 mg/ml. In another embodiment, the concentration of the anti-TIGIT antibody or antigen binding fragment thereof is about 12.5 mg/ml. In another embodiment, the concentration of the anti-TIGIT antibody or antigen binding fragment thereof is about 20 mg/ml. In another embodiment, the concentration of the anti-TIGIT antibody or antigen binding fragment thereof is about 25 mg/ml. In another embodiment, the concentration of the anti-TIGIT antibody or antigen binding fragment thereof is about 50 mg/ml. In another embodiment, the concentration of the anti-TIGIT antibody or antigen binding fragment thereof is about 75 mg/ml. In another embodiment, the concentration of the anti-TIGIT antibody or antigen binding fragment thereof is about or 100 mg/ml.

In some embodiments of the co-formulation, the concentration of the anti-human PD-1 antibody is from about 1 mg/ml to about 100 mg/ml. In one embodiments of the co-formulation, the concentration of the anti-human PD-1 antibody is from about 10 mg/ml to about 100 mg/ml. In another embodiment, the concentration of the anti-human PD-1 antibody is 20 mg/ml. In another embodiment, the concentration of the anti-human PD-1 antibody is 25 mg/ml.

In one embodiment, the co-formulation comprises about 20 mg/ml of the anti-PD1 antibody, about 20 mg/ml of the anti-TIGIT antibody, 10 mM L-histidine buffer, about 7% w/v sucrose, about 0.02% w/v polysorbate 80, and about 10 mM L-methionine.

In one embodiment, the co-formulation comprises about 25 mg/ml of the anti-PD1 antibody, about 25 mg/ml of the anti-TIGIT antibody, 10 mM L-histidine buffer, about 7% w/v sucrose, about 0.02% w/v polysorbate 80, and about 10 mM L-methionine.

In one embodiment, the co-formulation comprises about 50 mg/ml of the anti-PD1 antibody, about 50 mg/ml of the anti-TIGIT antibody, 10 mM L-histidine buffer, about 7% w/v sucrose, about 0.02% w/v polysorbate 80, and about 10 mM L-methionine.

In one aspect of any of the formulations described above, the formulation comprises an anti-TIGIT antibody or antigen-binding fragment thereof comprising three light chain CDRs and three heavy chain CDRs, wherein the light chain CDRs comprise CDRL1 of SEQ ID NO: 111 or variant thereof, CDRL2 of SEQ ID NO: 112 or variant thereof, CDRL3 of SEQ ID NO: 113 or variant thereof and the heavy chain CDRs comprise CDRH1 of SEQ ID NO: 108 or variant thereof, CDRH2 of SEQ ID NO: 154 or variant thereof, and CDHR3 of SEQ ID NO: 110 or variant thereof. In one aspect of any of the formulations described above, the formulation comprises an anti-TIGIT antibody or antigen-binding fragment thereof comprising three light chain CDRs and three heavy chain CDRs, wherein the light chain CDRs comprise CDRL1 of SEQ ID NO: 111, CDRL2 of SEQ ID NO: 112, CDRL3 of SEQ ID NO: 113 and the heavy chain CDRs comprise CDRH1 of SEQ ID NO: 108, CDRH2 of SEQ ID NO: 154, and CDHR3 of SEQ ID NO: 110. In another aspect, the formulation comprises an anti-TIGIT antibody or antigen binding fragment thereof comprising a heavy chain variable region comprising SEQ ID NO: 148 or variant thereof and a light chain variable region comprising SEQ ID NO: 152 or variant thereof. In another aspect, the formulation comprises an anti-TIGIT antibody or antigen binding fragment thereof comprising a heavy chain variable region comprising SEQ ID NO: 148 and a light chain variable region comprising SEQ ID NO: 152. In one aspect, the anti-TIGIT antibody or antigen binding fragment thereof further comprises a human heavy chain IgG1 constant domain comprising the amino acid sequence of SEQ ID NO:291 or variant thereof and a human kappa light chain constant domain comprising the amino acid sequence of SEQ ID NO:293 or variant thereof. In one aspect, the anti-TIGIT antibody or antigen binding fragment thereof further comprises a human heavy chain IgG1 constant domain comprising the amino acid sequence of SEQ ID NO:291 and a human kappa light chain constant domain comprising the amino acid sequence of SEQ ID NO:293. In another aspect, the anti-TIGIT antibody or antigen binding fragment thereof further comprises a human heavy chain IgG4 constant domain comprising the amino acid sequence of SEQ ID NO:292 or variant thereof and a human kappa light chain constant domain comprising the amino acid sequence of SEQ ID NO:293 or variant thereof. In another aspect, the anti-TIGIT antibody or antigen binding fragment thereof further comprises a human heavy chain IgG4 constant domain comprising the amino acid sequence of SEQ ID NO:292 and a human kappa light chain constant domain comprising the amino acid sequence of SEQ ID NO:293.

In one aspect of any of the formulations described above, the anti-human PD-1 antibody or antigen binding fragment thereof comprises three light chain CDRs and three heavy chain CDRs, wherein the light chain CDRs comprise CDRL1 of SEQ ID NO: 1 or variant thereof, CDRL2 of SEQ ID NO:2 or variant thereof, CDRL3 of SEQ ID NO:3 or variant thereof and the heavy chain CDRs comprise CDRH1 of SEQ ID NO: 6 or variant thereof, CDRH2 of SEQ ID NO: 7 or variant thereof, and CDHR3 of SEQ ID NO: 8 or variant thereof. In one aspect of any of the formulations described above, the anti-human PD-1 antibody or antigen binding fragment thereof comprises three light chain CDRs and three heavy chain CDRs, wherein the light chain CDRs comprise CDRL1 of SEQ ID NO: 1, CDRL2 of SEQ ID NO:2, CDRL3 of SEQ ID NO:3 and the heavy chain CDRs comprise CDRH1 of SEQ ID NO: 6, CDRH2 of SEQ ID NO: 7, and CDHR3 of SEQ ID NO: 8. In another aspect, the formulations comprise an anti-human PD1 antibody or antigen binding fragment thereof comprising a light chain variable region comprising SEQ ID NO: 4 or variant thereof and a heavy chain variable region comprising SEQ ID NO: 9 or variant thereof. In another aspect, the formulations comprise an anti-human PD1 antibody or antigen binding fragment thereof comprising a light chain variable region comprising SEQ ID NO: 4 and a heavy chain variable region comprising SEQ ID NO: 9. In another aspect, the formulations comprise an anti-human PD1 antibody or antigen binding fragment thereof comprising a light chain comprising SEQ ID NO: 5 and a heavy chain comprising SEQ ID NO: 10. In another aspect, the formulations comprise an anti-human PD1 antibody or antigen binding fragment thereof comprising a light chain comprising SEQ ID NO: 5 or variant thereof and a heavy chain comprising SEQ ID NO: 10 or variant thereof. In one aspect of any of the formulations described above, the anti-human PD-1 antibody or antigen binding fragment thereof is pembrolizumab. In another aspect, the anti-human PD-1 antibody or antigen binding fragment thereof is nivolumab.

In one aspect of any of the co-formulations described above, the formulation comprises (i) an anti-TIGIT antibody or antigen-binding fragment thereof comprising three light chain CDRs and three heavy chain CDRs, wherein the light chain CDRs comprise CDRL1 of SEQ ID NO: 111, CDRL2 of SEQ ID NO:112, CDRL3 of SEQ ID NO:113 and the heavy chain CDRs comprise CDRH1 of SEQ ID NO: 108, CDRH2 of SEQ ID NO: 154, and CDHR3 of SEQ ID NO: 110 and (ii) an anti-human PD-1 antibody or antigen binding fragment thereof comprising three light chain CDRs and three heavy chain CDRs, wherein the light chain CDRs comprise CDRL1 of SEQ ID NO: 1, CDRL2 of SEQ ID NO:2, CDRL3 of SEQ ID NO:3 and the heavy chain CDRs comprise CDRH1 of SEQ ID NO: 6, CDRH2 of SEQ ID NO: 7, and CDHR3 of SEQ ID NO: 8.

In one aspect of any of the above co-formulations, the formulation comprises (i) an anti-TIGIT antibody or antigen binding fragment thereof comprising a heavy chain variable region comprising SEQ ID NO: 148 and a light chain variable region comprising SEQ ID NO: 152 and (ii) an anti-human PD1 antibody or antigen binding fragment thereof comprising a light chain variable region comprising SEQ ID NO: 4 and a heavy chain variable region comprising SEQ ID NO: 9.

In another aspect of any of the above co-formulations, the formulation comprises (i) an anti-TIGIT antibody or antigen binding fragment thereof comprising a heavy chain variable region comprising SEQ ID NO: 148 and further comprising a human heavy chain IgG1 constant domain comprising the amino acid sequence of SEQ ID NO:291 and a light chain variable region comprising SEQ ID NO: 152 and further comprising a human kappa light chain constant domain comprising the amino acid sequence of SEQ ID NO:293 and (ii) an anti-human PD1 antibody or antigen binding fragment thereof comprising a light chain comprising SEQ ID NO: 5 and a heavy chain comprising SEQ ID NO: 10.

In another aspect of any of the above co-formulations, the formulation comprises (i) an anti-TIGIT antibody or antigen binding fragment thereof comprising a heavy chain variable region comprising SEQ ID NO: 148 and further comprising a human heavy chain IgG1 constant domain comprising the amino acid sequence of SEQ ID NO:292 and a light chain variable region comprising SEQ ID NO: 152 and further comprising a human kappa light chain constant domain comprising the amino acid sequence of SEQ ID NO:293 and (ii) an anti-human PD1 antibody or antigen binding fragment thereof comprising a light chain comprising SEQ ID NO: 5 and a heavy chain comprising SEQ ID NO: 10.

In one embodiment of any of the formulations described above, the formulation is contained in a glass vial. In another embodiment, the formulation is contained in an injection device. In another embodiment, the formulation is a liquid formulation. In one aspect, the formulation is frozen to at least below −70° C. In another embodiment, the formulation is a reconstituted solution from a lyophilized formulation.

The present invention provides a method of treating chronic infection or cancer in a mammalian subject (e.g. a human) in need thereof comprising: administering an effective amount of the anti-TIGIT formulation or the co-formulation set forth herein.

### BRIEF DESCRIPTION OF THE DRAWINGS

FIG. 1 shows the pH stability of the formulations over 9 months at various storage conditions.

FIG. 2 shows the polysorbate 80 concentration stability of the formulations over 9 months at various storage conditions.

FIG. 3 shows the potency by ELISA stability data for the formulations over 9 months at various storage conditions.

FIG. 4 shows the monomer (%) by UP-SEC stability data for the formulations over 9 months at various storage conditions.

FIG. 5 shows the high molecular weight (HMW) species (%) by UP-SEC stability data for the formulations over 9 months at various storage conditions.

FIG. 6 shows the low molecular weight (LMW) species (%) by UP-SEC stability data for the formulations over 9 months at various storage conditions.

FIG. 7 shows the purity heavy chain+light chain (%) by CE-SDS Reducing stability data for the formulations over 9 months at various storage conditions.

FIG. 8 shows the purity intact IgG (%) by CE-SDS Non-reducing stability data for the formulations over 9 months at various storage conditions.

### DETAILED DESCRIPTION OF THE INVENTION

In one aspect, the invention provides formulations comprising anti-TIGIT antibodies and antigen-binding fragments thereof comprising methionine. Also provided are co-formulations of an anti-TIGIT antibody or antigen binding fragment thereof and an anti-human PD-1 antibody or antigen binding fragment thereof comprising methionine. In each case, the formulation and co-formulation optionally comprises a chelating agent.

### I. DEFINITIONS AND ABBREVIATIONS

As used throughout the specification and appended claims, the following abbreviations apply:
API active pharmaceutical ingredient
CDR complementarity determining region in the immunoglobulin variable regions, defined using the Kabat numbering system,
unless otherwise indicated
CHO Chinese hamster ovary
CI confidence interval
DTPA diethylenetriaminepentaacetic acid
EC50 concentration resulting in 50% efficacy or binding
ELISA enzyme-linked immunosorbant assay
FFPE formalin-fixed, paraffin-embedded
FR framework region
HRP horseradish peroxidase
HNSCC head and neck squamous cell carcinoma
IC50 concentration resulting in 50% inhibition
IgG immunoglobulin G
IHC immunohistochemistry or immunohistochemical
mAb monoclonal antibody
MES 2-(N-morpholino)ethanesulfonic acid
NCBI National Center for Biotechnology Information
NSCLC non-small cell lung cancer
PCR polymerase chain reaction
PD-1 programmed death 1 (a.k.a. programmed cell death-1 and programmed death receptor 1)
PD-L1 programmed cell death 1 ligand 1
PD-L2 programmed cell death 1 ligand 2
PS80 polysorbate 80
TNBC triple negative breast cancer
V_{H} immunoglobulin heavy chain variable region
VK immunoglobulin kappa light chain variable region
V_{L} immunoglobulin light chain variable region
v/v volume per volume
WFI water for injection
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a formulation of the invention to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, inhibiting the growth or survival of tumor cells, eliminating or reducing the size of one or more cancerous lesions or tumors, decreasing the level of one or more tumor markers, ameliorating, reducing the severity or duration of cancer, prolonging the survival of a patient relative to the expected survival in a similar untreated patient.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

Positive therapeutic effects in cancer can be measured in a number of ways (See, W. A. Weber, J Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by administration of a formulation of the invention is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the formulations, treatment methods, and uses of the present invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient.

The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic antibody.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th}ed.; NIH Publ. No. 91-3242 (1991); Kabat (1978) Adv. Prot. Chem. 32:1-75; Kabat, et al., (1977) J Biol. Chem. 252:6609-6616; Chothia, et al., (1987) J Mol. Biol. 196:901-917 or Chothia, et al., (1989) Nature 342:878-883.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human TIGIT or human PD-1, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

The term “pharmaceutically effective amount” or “effective amount” means an amount whereby sufficient therapeutic composition or formulation is introduced to a patient to treat a diseased or condition. One skilled in the art recognizes that this level may vary according the patient's characteristics such as age, weight, etc.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

As used herein, “x % (w/v)” is equivalent to x g/100 ml (for example 5% w/v equals 50 mg/ml).

Formulations of the present invention include antibodies and fragments thereof that are biologically active when reconstituted or in liquid form.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Chothia” means an antibody numbering system described in Al-Lazikani et al., JMB 273:927-948 (1997).

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

A “growth inhibitory agent” when used herein refers to a compound or composition which inhibits growth of a cell, especially cancer cell over expressing any of the genes identified herein, either in vitro or in vivo. Thus, the growth inhibitory agent is one which significantly reduces the percentage of cells over expressing such genes in S phase. Examples of growth inhibitory agents include agents that block cell cycle progression (at a place other than S phase), such as agents that induce Gi arrest and M-phase arrest. Classical M-phase blockers include the vincas (vincristine and vinblastine) taxanes, and topo II inhibitors such as doxorubicin, epirubicin, daunorubicin, and etoposide. Those agents that arrest Gi also spill over into S-phase arrest, for example, DNA alkylating agents such as dacarbazine, mechlorethamine, and cisplatin. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995).

The terms “TIGIT binding fragment,” “antigen binding fragment thereof,”, “binding fragment thereof” or “fragment thereof” encompass a fragment or a derivative of an antibody that still substantially retains its biological activity of binding to antigen (human TIGIT) and inhibiting its activity (e.g., blocking the binding of human TIGIT to its native ligands).

Therefore, the term “antibody fragment” or TIGIT binding fragment refers to a portion of a full length antibody, generally the antigen binding or variable region thereof. Examples of TIGIT antibody fragments include Fab, Fab′, F(ab′)₂, and Fv fragments. Typically, a binding fragment or derivative retains at least 10% of its TIGIT inhibitory activity. In some embodiments, a binding fragment or derivative retains at least 25%, 50%, 60%, 70%, 80%, 90%, 95%, 99% or 100% (or more) of its TIGIT inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 10⁹ liters/mol, as determined, e.g., by Scatchard analysis. Munsen et al. (1980) Analyt. Biochem. 107:220-239. It is also intended that a TIGIT binding fragment can include variants having conservative amino acid substitutions that do not substantially alter its biologic activity.

The terms “PD-1 binding fragment,” “antigen binding fragment thereof,” “binding fragment thereof” or “fragment thereof” encompass a fragment or a derivative of an antibody that still substantially retains its biological activity of binding to antigen (human PD-1) and inhibiting its activity (e.g., blocking the binding of PD-1 to PDL1 and PDL2). Therefore, the term “antibody fragment” or PD-1 binding fragment refers to a portion of a full length antibody, generally the antigen binding or variable region thereof. Examples of antibody fragments include Fab, Fab′, F(ab′)₂, and Fv fragments. Typically, a binding fragment or derivative retains at least 10% of its PD-1 inhibitory activity. In some embodiments, a binding fragment or derivative retains at least 25%, 50%, 60%, 70%, 80%, 90%, 95%, 99% or 100% (or more) of its PD-1 inhibitory activity, although any binding fragment with sufficient affinity to exert the desired biological effect will be useful. In some embodiments, an antigen binding fragment binds to its antigen with an affinity that is at least two fold greater, preferably at least ten times greater, more preferably at least 20-times greater, and most preferably at least 100-times greater than the affinity with unrelated antigens. In one embodiment the antibody has an affinity that is greater than about 10⁹ liters/mol, as determined, e.g., by Scatchard analysis. Munsen et al. (1980) Analyt. Biochem. 107:220-239. It is also intended that a PD-1 binding fragment can include variants having conservative amino acid substitutions that do not substantially alter its biologic activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Cin. Immunol. 116:731 at 734-35.

“Fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only. A fully human antibody may be generated in a human being, in a transgenic animal having human immunoglobulin germline sequences, by phage display or other molecular biological methods.

“Hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J Mol. Biol. 196: 901-917). As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

“Conservatively modified variants” or “conservative substitution” refers to substitutions of amino acids are known to those of skill in this art and may be made generally without altering the biological activity of the resulting molecule, even in essential regions of the polypeptide. Such exemplary substitutions are preferably made in accordance with those set forth in Table 1 as follows:

TABLE 1
Exemplary Conservative Amino Acid Substitutions

| Original | Conservative |
|---|---|
| residue | substitution |
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J Allergy Clin. Immunol. 116:731.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

The term “buffer” encompasses those agents which maintain the solution pH of the formulations of the invention in an acceptable range, or, for lyophilized formulations of the invention, provide an acceptable solution pH prior to lyophilization.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A “reconstituted” formulation is one that has been prepared by dissolving a lyophilized protein formulation in a diluent such that the protein is dispersed in the reconstituted formulation. The reconstituted formulation is suitable for administration, e.g. parenteral administration), and may optionally be suitable for subcutaneous administration.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e. % high molecular weight species as determined, for example, by HP-SEC.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

### II. FORMULATIONS AND CO-FORMULATIONS OF THE INVENTION

In one aspect, the invention provides biological formulations comprising anti-TIGIT antibodies or antigen binding fragments thereof which specifically bind to human TIGIT as the active pharmaceutical ingredient. Inclusion of methionine in such formulations reduces the oxidation of the methionine residues in the Fc region of the anti-TIGIT antibody and, in the example of an anti-TIGIT antibody comprising a CDRH3 of SEQ ID NO: 110, the tryoptophan. Such formulations may further comprise a chelator, such as, DTPA, which can further reduce oxidation.

In one aspect, the invention also provides a co-formulation of an anti-TIGIT antibody with an anti-PD-1 antibody. The major degradation pathways of pembrolizumab included oxidation of methionine 105 (Met105) in the heavy chain CDR3 (e.g., M105 of SEQ ID NO: 10) upon peroxide stress and oxidation of Met105 and Fc methionine residues when exposed to light. Pembrolizumab maintained its bioactivity under most stress conditions for the degradation levels tested. However, reduction in affinity to PD-1 was observed for peroxide stressed samples by Surface Plasmon Resonance (SPR). An exposed methionine residue or a methionine residue in the CDR of an antibody has the potential of impacting the biological activity of the antibody through oxidation. The addition of methionine is able to reduce oxidation of Met105 within the pembrolizumab heavy chain CDR.

### Anti-PD-1 Antibodies and Antigen-Binding Fragments Thereof

In one aspect, the invention provides stable biological formulations comprising anti-TIGIT antibodies or antigen binding fragments thereof, co-formulated with an anti-human PD-1 antibodies or antigen binding fragments thereof which specifically bind to human PD-1 (e.g. a human or humanized anti-PD-1 antibody) as the active pharmaceutical ingredient (PD-1 API), as well as methods for using the formulations of the invention. Any anti-PD-1 antibody or antigen binding fragment thereof can be used in the co-formulations and methods of the invention. In particular embodiments, the PD-1 API is an anti-PD-1 antibody, which is selected from pembrolizumab and nivolumab. In specific embodiments, the anti-PD-1 antibody is pembrolizumab. In alternative embodiments, the anti-PD-1 antibody is nivolumab. Table 2 provides amino acid sequences for exemplary anti-human PD-1 antibodies pembrolizumab and nivolumab. Alternative PD-1 antibodies and antigen-binding fragments that are useful in the co-formulations and methods of the invention are shown in Table 3.

In some embodiments, an anti-human PD-1 antibody or antigen binding fragment thereof for use in the co-formulations of the invention comprises three light chain CDRs of CDRL1, CDRL2 and CDRL3 and/or three heavy chain CDRs of CDRH1, CDRH2 and CDRH3.

In one embodiment of the invention, CDRL1 is SEQ ID NO:1 or a variant of SEQ ID NO:1, CDRL2 is SEQ ID NO:2 or a variant of SEQ ID NO:2, and CDRL3 is SEQ ID NO:3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO:8.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO:13.

In one embodiment, CDRH1 is SEQ ID NO:16 or a variant of SEQ ID NO:16, CDRH2 is SEQ ID NO:17 or a variant of SEQ ID NO:17, and CDRH3 is SEQ ID NO:18 or a variant of SEQ ID NO:18.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO:8.

In an alternative embodiment, the three light chain CDRs are SEQ ID NO:11, SEQ ID NO:12, and SEQ ID NO:13 and the three heavy chain CDRs are SEQ ID NO:16, SEQ ID NO:17 and SEQ ID NO:18.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO:24, CDRH2 is SEQ ID NO: 25 or a variant of SEQ ID NO:25, and CDRH3 is SEQ ID NO:26 or a variant of SEQ ID NO:26.

In another embodiment, the three light chain CDRs are SEQ ID NO:21, SEQ ID NO:22, and SEQ ID NO:23 and the three heavy chain CDRs are SEQ ID NO:24, SEQ ID NO:25 and SEQ ID NO:26.

Some anti-human PD-1 antibody and antigen binding fragments of the invention comprise alight chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO:14 or a variant of SEQ ID NO:14, and the heavy chain variable region comprises SEQ ID NO:19 or a variant of SEQ ID NO:19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO:29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the co-formulations of the invention, the anti-human PD-1 antibody or antigen binding fragment comprises a light chain variable region comprising or consisting of SEQ ID NO:4 and a heavy chain variable region comprising or consisting SEQ ID NO:9. In a further embodiment, the anti-human PD-1 antibody or antigen binding fragment comprises a light chain variable region comprising or consisting of SEQ ID NO:14 and a heavy chain variable region comprising or consisting of SEQ ID NO:19. In one embodiment of the formulations of the invention, the anti-human PD-1 antibody or antigen binding fragment comprises a light chain variable region comprising or consisting of SEQ ID NO:28 and a heavy chain variable region comprising or consisting SEQ ID NO:27. In a further embodiment, the anti-human PD-1 antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO:27.

In another embodiment, the co-formulations of the invention comprise an anti-human PD-1 antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment, the anti-human PD-1 antibody or antigen binding protein of the co-formulations of the invention comprises V_{L} and V_{H} domains having up to 1, 2, 3, 4, or 5 or more amino acid substitutions, and exhibits specific binding to PD-1.

In any of the embodiments above, the PD-1 API may be a full-length anti-PD-1 antibody or an antigen binding fragment thereof that specifically binds human PD-1. In certain embodiments, the PD-1 API is a full-length anti-PD-1 antibody selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the PD-1 API is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the PD-1 API is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 API is an anti-PD-1 antibody comprising a light chain comprising or consisting of a sequence of amino acid residues as set forth in SEQ ID NO:32 and a heavy chain comprising or consisting of a sequence of amino acid residues asset forth in SEQ ID NO:31. In additional embodiments, the PD-1 API is an anti-PD-1 antibody comprising a light chain comprising or consisting of a sequence of amino acid residues as set forth in SEQ ID NO:33 and a heavy chain comprising or consisting of a sequence of amino acid residues asset forth in SEQ ID NO:31. In yet additional embodiments, the PD-1 API is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some co-formulations of the invention, the PD-1 API is pembrolizumab or a pembrolizumab biosimilar. In some co-formulations of the invention, the PD-1 API is nivolumab or a nivolumab biosimilar.

Ordinarily, amino acid sequence variants of the anti-PD-1 antibodies and antigen binding fragments of the invention and the anti-TIGIT antibodies and antigen binding fragments will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 2
Exemplary PD-1 Antibody Sequences

| | | |
|---|---|---|
| Antibody | | |
| Feature | Amino Acid Sequence | SEQ ID NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWYQQKPGQA | 4 |
| Region | PRLLIYLASYLESGVPARFSGSGSGTDFTLTISSLEPEDFAVYYCQH | |
| | SRDLPLTFGGGTKVEIK | |
| Light Chain | EIVLTQSPATLSLSPGERATLSCRASKGVSTSGYSYLHWYQQKPGQA | 5 |
| | PRLLIYLASYLESGVPARFSGSGSGTDFTLTISSLEPEDFAVYYCQH | |
| | SRDLPLTFGGGTKVEIKRTVAAPSVFIFPPSDEQLKSGTASVVCLLN | |
| | NFYPREAKVQWKVDNALQSGNSQESVTEQDSKDSTYSLSSTLTLSKA | |
| | DYEKHKVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWVRQAPGQGLEW | 9 |
| Region | MGGINPSNGGTNFNEKFKNRVTLTTDSSTTTAYMELKSLQFDDTAVY | |
| | YCARRDYRFDMGFDYWGQGTTVTVSS | |
| Heavy Chain | QVQLVQSGVEVKKPGASVKVSCKASGYTFTNYYMYWVRQAPGQGLEW | 10 |
| | MGGINPSNGGTNFNEKFKNRVTLTTDSSTTTAYMELKSLQFDDTAVY | |
| | YCARRDYRFDMGFDYWGQGTTVTVSSASTKGPSVFPLAPCSRSTSES | |
| | TAALGCLVKDYFPEPVTVSWNSGALTSGVHTFPAVLQSSGLYSLSSV | |
| | VTVPSSSLGTKTYTCNVDHKPSNTKVDKRVESKYGPPCPPCPAPEFL | |
| | GGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLP | |
| | SSIEKTISKAKGQPREPQVYTLPPSQEEMTKNQVSLTCLVKGFYPSD | |
| | IAVEWESNGQPENNYKTTPPVLDSDGSFFLYSRLTVDKSRWQEGNVF | |
| | SCSVMHEALHNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWYQQKPGQAPRLL | 14 |
| Region | IYDASNRATGIPARFSGSGSGTDFTLTISSLEPEDFAVYYCQQSSNW | |
| | PRTFGQGTKVEIK | |
| Light Chain | EIVLTQSPATLSLSPGERATLSCRASQSVSSYLAWYQQKPGQAPRLL | 15 |
| | IYDASNRATGIPARFSGSGSGTDFTLTISSLEPEDFAVYYCQQSSNV | |
| | VPRTFGQGTKVEIKRTVAAPSVFIFPPSDEQLKSGTASVVCLLNNFY | |
| | PREAKVQWKVDNALQSGNSQESVTEQDSKDSTYSLSSTLTLSKADYE | |
| | KHKVYACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHWVRQAPGKGLEW | 19 |
| Region | VAVIWYDGSKRYYADSVKGRFTISRDNSKNTLFLQMNSLRAEDTAVY | |
| | YCATNDDYWGQGTLVTVSS | |
| Heavy Chain | QVQLVESGGGVVQPGRSLRLDCKASGITFSNSGMHWVRQAPGKGLEW | 20 |
| | VAVIWYDGSKRYYADSVKGRFTISRDNSKNTLFLQMNSLRAEDTAVY | |
| | YCATNDDYWGQGTLVTVSSASTKGPSVFPLAPCSRSTSESTAALGCL | |
| | VKDYFPEPVTVSWNSGALTSGVHTFPAVLQSSGLYSLSSVVTVPSSS | |
| | LGTKTYTCNVDHKPSNTKVDKRVESKYGPPCPPCPAPEFLGGPSVFL | |
| | FPPKPKDTLMISRTPEVTCVVVDVSQEDPEVQFNWYVDGVEVHNAKT | |
| | KPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMHE | |
| | ALHNHYTQKSLSLSLGK | |

TABLE 3
Additional PD-1 Antibodies and Antigen Binding Fragments Useful
in the Co-Formulations, Methods and Uses of the Invention.
A. Antibodies and antigen binding fragments comprising
light and heavy chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

C. Antibodies and antigen binding fragments comprising
the mature h109A heavy chain variable region and one of
the mature K09A light chain variable regions in WO 2008/156712

| | |
|---|---|
| Heavy chain VR | SEQ ID NO: 27 |
| Light chain VR | SEQ ID NO: 28 or SEQ ID NO: 29 or SEQ ID NO: 30 |

D. Antibodies and antigen binding fragments comprising
the mature 409 heavy chain and one of the mature
K09A light chains in WO 2008/156712

| | |
|---|---|
| Heavy chain | SEQ ID NO: 31 |
| Light chain | SEQ ID NO: 32 or SEQ ID NO: 33 or SEQ ID NO: 34 |

In some embodiments of the co-formulation of the invention, the PD-1 API (i.e. the anti-PD-1 antibody or antigen binding fragment thereof) is present in a concentration of from about 25 mg/mL to about 100 mg/mL. In alternative embodiments, the API is present in a concentration of about 10 mg/mL, about 25 mg/mL, about 50 mg/mL, about 75 mg/mL, or about 100 mg/mL.

### Anti-TIGIT Antibodies and Antigen-Binding Fragment Thereof

In one aspect, the invention provides biological formulations comprising anti-TIGIT antibodies or antigen binding fragments thereof which specifically bind to human TIGIT (e.g. a human or humanized anti-TIGIT antibody) as the active pharmaceutical ingredient (TIGIT API), as well as methods for using the formulations of the invention.

In another aspect, the invention also provides biological co-formulations comprising (i) anti-TIGIT antibody or antigen binding fragment thereof which specifically bind to human TIGIT (e.g. a human or humanized anti-TIGIT antibody) and (ii) an anti-human PD-1 antibody or antigen binding fragment thereof which specifically binds to human PD-1. Any anti-TIGIT antibody or antigen binding fragment thereof can be used in the formulation, including the co-formulation, and methods of the invention. Exemplary anti-TIGIT antibody sequences are set forth below in Tables 4 and 5.

TABLE 4
Exemplary anti-TIGIT antibodies

| Description | SEQ ID NO: | SEQUENCE |
|---|---|---|
| 14A6 H-CDR1 | 35 | SDYWG |
| 14A6 H-CDR2 | 36 | FITYSGSTSYNPSLKS |
| 14A6 H-CDR3 | 37 | MPSFITLASLSTWEGYFDF |
| 14A6 L-CDR1 | 38 | KASQSIHKNLA |
| 14A6 L-CDR2 | 39 | YANSLQT |
| 14A6 L-CDR3 | 40 | QQYYSGWT |
| 14A6 PARENTAL VH | 41 | EVQLQESGPGLVKPSQSLSLTCSVTGSSIASDYWGWIR |
| | | KFPGNKMEWMGFITYSGSTSYNPSLKSRISITRDTSKN |
| | | QFFLQLHSVTTDDTATYSCARMPSFITLASLSTWEGYF |
| | | DFWGPGTMVTVSS |
| 14A6 PARENTAL VL | 42 | DIQMTQSPSLLSASVGDRVTLNCKASQSIHKNLAWYQQ |
| | | KLGEAPKFLIYYANSLQTGIPSRFSGSGSGTDFTLTIS |
| | | GLQPEDVATYFCQQYYSGWTFGGGTKVELK |
| Hu14A6VH.1 | 43 | EVQLQESGPGLVKPSETLSLTCTVSGGSISSDYWGWIR |
| | | QPPGKGLEWIGFITYSGSTSYNPSLKSRVTISVDTSKN |
| | | QFSLKLSSVTAADTAVYYCARMPSFITLASLSTWEGYF |
| | | DFWGQGTMVTVSS |
| Hu14A6VH.1a | 44 | EVQLQESGPGLVKPSETLSLTCTVSGGSISSDYWGWIR |
| | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| Hu14A6VH.1b | 45 | EVQLQESGPGLVKPSETLSLTCTVSGGSISSDYWGWIR |
| | | QPPGKGLEWIGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.1c | 46 | EVQLQESGPGLVKPSETLSLTCTVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.1d | 47 | EVQLQESGPGLVKPSETLSLTCTVSGGSISSDYWGWIR |
| | | QPPGKGLEWIGFITYSGSTSYNPSLKSRVTISRDTSKN |
| | | QFSLKLHSVTAADTAVYYCARMPSFITLASLSTWEGYF |
| | | DFWGQGTMVTVSS |
| Hu14A6VH.1e | 48 | EVQLQESGPGLVKPSETLSLTCTVSGGSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.1f | 49 | EVQLQESGPGLVKPSETLSLTCTVSGSSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.1g | 50 | EVQLQESGPGLVKPSETLSLTCTVSGSSISSDYWGWIR |
| | | QPPGKGLEWMGFITYSGSTSYNPSLKSRITISVDTSKN |
| | | QFSLKLHSVTAADTAVYYCARMPSFITLASLSTWEGYF |
| | | DFWGQGTMVTVSS |
| Hu14A6VH.2 | 51 | EVQLQESGPGLVKPSETLSLTCAVSGYSISSDYWGWIR |
| | | QPPGKGLEWIGFITYSGSTSYNPSLKSRVTISVDTSKN |
| | | QFSLKLSSVTAADTAVYYCARMPSFITLASLSTWEGYF |
| | | DFWGQGTMVTVSS |
| Hu14A6VH.2a | 52 | EVQLQESGPGLVKPSETLSLTCAVSGYSISSDYWGWIR |
| | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| Hu14A6VH.2b | 53 | EVQLQESGPGLVKPSETLSLTCAVSGYSISSDYWGWIR |
| | | QPPGKGLEWIGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.2c | 54 | EVQLQESGPGLVKPSETLSLTCAVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.2d | 55 | EVQLQESGPGLVKPSETLSLTCAVSGYSISSDYWGWIR |
| | | QPPGKGLEWIGFITYSGSTSYNPSLKSRVTISRDTSKN |
| | | QFSLKLHSVTAADTAVYYCARMPSFITLASLSTWEGYF |
| | | DFWGQGTMVTVSS |
| Hu14A6VH.2e | 56 | EVQLQESGPGLVKPSETLSLTCAVSGYSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.2f | 57 | EVQLQESGPGLVKPSETLSLTCAVSGSSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.2e | 58 | EVQLQESGPGLVKPSETLSLTCAVSGSSISSDYWGWIR |
| | | QPPGKGLEWMGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6Vk.1 | 59 | DIQMTQSPSSLSASVGDRVTITCKASQSIHKNLAWYQQ |
| | | KPGKAPKLLIYYANSLQTGVPSRFSGSGSGTDFTLTIS |
| | | SLQPEDFATYYCQQYYSGWTFGGGTKVEIK |
| Hu14A6Vk.1a | 60 | DIQMTQSPSSLSASVGDRVTITCKASQSIHKNLAWYQQ |
| | | KPGKAPKFLIYYANSLQTGVPSRFSGSGSGTDFTLTIS |
| | | SLQPEDFATYYCQQYYSGWTFGGGTKVEIK |
| Hu14A6Vk.1b | 61 | DIQMTQSPSSLSASVGDRVTITCKASQSIHKNLAWYQQ |
| | | KPGKAPKFLIYYANSLQTGIPSRFSGSGSGTDFTLTIS |
| | | SLQPEDFATYYCQQYYSGWTFGGGTKVEIK |
| Hu14A6Vk.2 | 62 | DIQMTQSPSSLSASVGDRVTITCKASQSIHKNLAWYQQ |
| | | KPGKVPKLLIYYANSLQTGVPSRFSGSGSGTDFTLTIS |
| | | SLQPEDVATYYCQQYYSGWTFGGGTKVEIK |
| Hu14A6Vk.2a | 63 | DIQMTQSPSSLSASVGDRVTITCKASQSIHKNLAWYQQ |
| | | KPGKVPKFLIYYANSLQTGVPSRFSGSGSGTDFTLTIS |
| | | SLQPEDVATYYCQQYYSGWTFGGGTKVEIK |
| Hu14A6Vk.2b | 64 | DIQMTQSPSSLSASVGDRVTITCKASQSIHKNLAWYQQ |
| | | KPGKVPKFLIYYANSLQTGIPSRFSGSGSGTDFTLTIS |
| | | SLQPEDVATYYCQQYYSGWTFGGGTKVEIK |
| 16AHA_tigit_14a6_humanized_VH1 | 65 | EVQLQESGPGLVKPSETLSLTCTVSGSSIASDYWGWIR |
| LB155.14A6.G2.A8_VH1 | | QPPGKGLEWIGFITYSGSTSYNPSLKSRVTISVDTSKN |
| | | QFSLKLSSVTAADTAVYYCARMPSFITLASLSTWEGYF |
| | | DFWGQGTMVTVSSAS |
| 18AHA_tigit_14a6_humanized_VH2 | 66 | EVQLQESGPGLVKPSETLSLTCTVSGSSIASDYWGWIR |
| LB155.14A6.G2.A8_VH2 | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 20AHA_tigit_14a6_humanized_VH3 | 67 | EVQLQESGPGLVKPSETLSLTCTVSGSSIASDYWGWIR |
| LB155.14A6.G2.A8_VH3 | | KPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 21AHA_tigit_14a6_humanized_VH4 | 68 | EVQLQESGPGLVKPSETLSLTCTVSGSSIASDYWGWIR |
| LB155.14A6.G2.A8_VH4 | | QPPGKKLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 19AHA_tigit_14a6_humanized_VH5 | 69 | EVQLQESGPGLVKPSETLSLTCTVSGSSIASDYWGWIR |
| LB155.14A6.G2.A8_VH5 | | QPPGKGMEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 22AHA_tigit_14a6_humanized_VH6 | 70 | EVQLQESGPGLVKPSETLSLTCTVSGSSIASDYWGWIR |
| LB155.14A6.G2.A8_VH6 | | KPPGKKMEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 23AHA_tigit_14a6_humanized_VH7 | 71 | EVQLQESGPGLVKPSETLSLTCTVSGSSIASDYWGWIR |
| LB155.14A6.G2.A8_VH7 | | QFPGKGLEWIGFITYSGSTSYNPSLKSRVTISRDTSKN |
| | | QFSLKLSSVTADDTAVYYCARMPSFITLASLSTWEGYF |
| | | DFWGQGTMVTVSS |
| 24AHA_tigit_14a6_humanized_VH8 | 72 | EVQLQESGPGLVKPSETLSLTCTVSGSSIASDYWGWIR |
| LB155.14A6.G2.A8_VH8 | | KPPGKKMEWIGFITYSGSTSYNPSLKSRVTISVDTSKN |
| | | QFSLKLSSVTAADTAVYYCARMPSFITLASLSTWEGYF |
| | | DFWGQGTMVTVSS |
| 25AHA_tigit_14a6_humanized_VH9 | 73 | EVQLQESGPGLVKPSETLSLTCSVTGSSIASDYWGWIR |
| LB155.14A6.G2.A8_VH9 | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 26AHA_tigit_14a6_humanized_VH10 | 74 | EVQLQQSGAGLLKPSETLSLTCSVTGSSIASDYWGWIR |
| LB155.14A6.G2.A8_VH10 | | QPPGKGLEWIGFITYSGSTSYNPSLKSRVTISVDTSKN |
| | | QFSLKLSSVTAADTAVYYCARMPSFITLASLSTWEGYF |
| | | DFWGQGTMVTVSS |
| 27AHA_tigit_14a6_humanized_VH11 | 75 | EVQLQESGPGLVKPPGTLSLTCSVTGSSIASDYWGWVR |
| LB155.14A6.G2.A8_VH11 | | QPPGKGLEWIGFITYSGSTSYNPSLKSRVTISVDTSKN |
| | | QFSLKLSSVTAADTAVYYCARMPSFITLASLSTWEGYF |
| | | DFWGQGTMVTVSS |
| 09AHA_tigit_14a6_humanized_VL1 | 76 | DIQMTQSPSSLSASVGDRVTITCKASQSIHKNLAWYQQ |
| LB155.14A6.G2.A8_VL1 | | KPGKAPKLLIYYANSLQTGVPSRFSGSGSGTDFTLTIS |
| | | SLQPEDFATYYCQQYYSGWTFGGGTKVEIK |
| 11AHA_tigit_14a6_humanized_VL2 | 77 | DIQMTQSPSSLSASVGDRVITCKASQSIHKNLAWYQQK |
| LB155.14A6.G2.A8_VL2 | | PGKAPKFLIYYANSLQTGVPSRFSGSGSGTDFTLTISS |
| | | LQPEDFATYYCQQYYSGWTFGGGTKVEIK |
| 12AHA_tigit_14a6_humanized_VL3 | 78 | DIQMTQSPSSLSASVGDRVTITCKASQSIHKNLAWYQQ |
| LB155.14A6.G2.A8_VL3 | | KPGKAPKLLIYYANSLQTGVPSRFSGSGSGTDFTLTIS |
| | | SLQPEDFATYFCQQYYSGWTFGGGTKVEIK |
| 13AHA_tigit_14a6_humanized_VL4 | 79 | DIQMTQSPSSLSASVGDRVTITCKASQSIHKNLAWYQQ |
| LB155.14A6.G2.A8_VL4 | | KPGKAPKFLIYYANSLQTGVPSRFSGSGSGTDFTLTIS |
| | | SLQPEDFATYFCQQYYSGWTFGGGTKVEIK |
| 15AHA_tigit_14a6_humanized_VL5 | 80 | DIQMTQSPSSLSASVGDRVTITCKASQSIHKNLAWYQQ |
| LB155.14A6.G2.A8_VL5 | | KPGKAPKLLIYYANSLQTGIPSRFSGSGSGTDFTLTIS |
| | | SLQPEDFATYYCQQYYSGWTFGGGTKVEIK |
| 28H5 H-CDR1 | 81 | GYSITSDYAWN |
| 28H5 H-CDR2 | 82 | YISNSGSASYNPSLKS |
| 28H5 H-CDR3 | 83 | LIYYDYGGAMNF |
| 28H5 L-CDR1 | 84 | KASQGVSTTVA |
| 28H5 L-CDR2 | 85 | SASYRYT |
| 28H5 L-CDR3 | 86 | QHYYSTPWT |
| 28H5 PARENTAL VH | 87 | DVQLQESGPGLVKPSQSLSLTCTVTGYSITSDYAWNWI |
| | | RQFPGNKLEWMGYISNSGSASYNPSLKSRISITRDTSK |
| | | NQFFLQLNSVTTEDTATYYCATLIYYDYGGAMNFWGQG |
| | | TSVTVSS |
| 28H5 PARENTAL VL | 88 | DIVMTQSHKFMSTSVGDRVSITCKASQGVSTTVAWYQQ |
| | | KPGQSPKLLIYSASYRYTGVPDRFTGSGSGTDFTFTIS |
| | | SVQSEDLAVYYCQHYYSTPWTFGGGTKLEIK |
| 14H6 L-CDR2 variant | 89 | YASNLQT |
| 14H6 L-CDR2 variant | 90 | YASSLQT |
| 14H6 L-CDR2 variant | 91 | YASTLQT |
| 14H6 L-CDR2 variant | 92 | YATTLQT |
| 14H6 L-CDR2 variant | 93 | YASYLQT |
| 14H6 L-CDR2 variant | 94 | YANQLQT |
| 14H6 L-CDR2 variant | 95 | YAGSLQT |
| 14H6 L-CDR2 variant | 96 | YASQLQT |
| 14H6 L-CDR2 variant | 97 | YADSLQT |
| 14H6 L-CDR3 variant | 98 | QQYYSGFT |
| 14H6 L-CDR3 variant | 99 | QQYYSGYT |
| 14H6 L-CDR3 variant | 100 | QQYYSGIT |
| 14H6 L-CDR3 variant | 101 | QQYYSGVT |
| 14H6 L-CDR3 variant | 102 | QQYYSGLT |
| 14H6 H-CDR3 variant | 103 | MPSFITLASLSTFEGYFDF |
| 14H6 H-CDR3 variant | 104 | MPSFITLASLSTYEGYFDF |
| 14H6 H-CDR3 variant | 105 | MPSFITLASLSTIEGYFDF |
| 14H6 H-CDR3 variant | 106 | MPSFITLASLSTVEGYFDF |
| 14H6 H-CDR3 variant | 107 | MPSFITLASLSTLEGYFDF |
| 31C6 H-CDR1 | 108 | SYVMH |
| 31C6 H-CDR2 | 109 | YIDPYNDGAKYNEKFKG |
| 31C6 H-CDR3 | 110 | GGPYGWYFDV |
| 31C6 L-CDRI | 111 | RASEHIYSYLS |
| 31C6 L-CDR2 | 112 | NAKTLAE |
| 31C6 L-CDR3 | 113 | QHHFGSPLT |
| 31C6 PARENTAL VH (with | 114 | EVQLQQSGPELVKPGSSVKMSCKASGYTFSSYVMHWVK |
| CDRs underlined) | | QKPGQGLEWIGYIDPYNDGAKYNEKFKGKATLTSDKSS |
| | | STAYMELSSLTSEDSAVYYCARGGPYGWYFDVWGAGTT |
| | | VTVSS |
| 31C6 PARENTAL VL (with | 115 | DIQMTQSPASLSASVGETVTITCRASEHIYSYLSWYQQ |
| CDRs underlined) | | KQGKSPQLLVYNAKTLAEGVPSRFSGSGSGTQFSLKIN |
| | | SLQPEDFGTYYCQHHFGSPLTFGAGTTLELK |
| 31C6 H-CDR2 VARIANT | 116 | YIDPYNrGAKYNEKFG |
| (D56R) | | |
| 31C6 H-CDR2 VARIANT | 117 | YIDPYNlGAKYNEKGF |
| (D56L) | | |
| 31C6 H-CDR2 VARIANT | 118 | YIDPYNkGAKYNEKFG |
| (D56K) | | |
| 31C6 H-CDR2 VARIANT | 119 | YIDPYNfGAKYNEKFG |
| (D56F) | | |
| 31C6 H-CDR2 VARIANT | 120 | YIDPYNsGAKYNEKFG |
| (D56S) | | |
| 31C6 H-CDR2 VARIANT | 121 | YIDPYNyGAKYNEKFG |
| (D56Y) | | |
| 31C6 H-CDR2 VARIANT | 122 | YIDPYNvGAKYNEKFG |
| (D56V) | | |
| 31C6 H-CDR2 VARIANT | 123 | YIDPYNDrAKYNEKFKG |
| (G57R) | | |
| 31C6 H-CDR2 VARIANT | 124 | YIDPYNDnAKYNEKFKG |
| (G57N) | | |
| 31C6 H-CDR2 VARIANT | 125 | YIDPYNDqAKYNEKFKG |
| (G57Q) | | |
| 31C6 H-CDR2 VARIANT | 126 | YIDPYNDeAKYNEKFKG |
| (G57E) | | |
| 31C6 H-CDR2 VARIANT | 127 | YIDPYND1AKYNEKFKG |
| (G57L) | | |
| 31C6 H-CDR2 VARIANT | 128 | YIDPYNDkAKYNEKFKG |
| (G57K) | | |
| 31C6 H-CDR2 VARIANT | 129 | YIDPYNDsAKYNEKFKG |
| (G57S) | | |
| 31C6 H-CDR2 VARIANT | 130 | YIDPYNDyAKYNEKFKG |
| (G57Y) | | |
| 31C6 H-CDR2 VARIANT | 131 | YIDPYNDvAKYNEKFKG |
| (G57V) | | |
| 31C6 L-CDR2 variant | 132 | AAKTLAE |
| (N50A) | | |
| 31C6 L-CDR2 variant | 133 | YAKTLAE |
| (N50Y) | | |
| 31C6 L-CDR2 variant | 134 | WAKTLAE |
| (N50W) | | |
| 31C6 L-CDR2 variant | 135 | SAKTLAE |
| (N50S) | | |
| 31C6 L-CDR2 variant | 136 | TAKTLAE |
| (N50T) | | |
| 31C6 L-CDR2 variant | 137 | IAKTLAE |
| (N50I) | | |
| 31C6 L-CDR2 variant | 138 | VAKTLAE |
| (N50V) | | |
| 31C6 L-CDR2 variant | 139 | NNKTLAE |
| (A51N) | | |
| 31C6 L-CDR2 variant | 140 | NIKTLAE |
| (A51I) | | |
| 31C6 L-CDR2 variant | 141 | NLLTLAE |
| (A51L) | | |
| 31C6 L-CDR2 variant | 142 | NTKTLAE |
| (A51T) | | |
| 31C6 L-CDR2 variant | 143 | NVKTLAE |
| (A51V) | | |
| 31C6_HUMZ_VH1 | 144 | EVQLVQSGAEVKKPGASVKVSCKASGYTFSSYVMHWVR |
| (with CDRs underlined) | | QAPGQRLEWIGYIDPYNDGAKYSQKFQGRVTLTRDTSA |
| | | STAYMELSSLRSEDTAVYYCARGGPYGWYFDVWGQGTT |
| | | VTVSS |
| 31C6_HUMZ_VH2 | 145 | EVQLVQSGAEVKKPGASVKVSCKASGYTFSSYVMHWVR |
| (with CDRs underlined) | | QAPGQRLEWIGYIDPYNDGAKYSQKFQGRVTLTSDKSA |
| | | STAYMELSSLRSEDTAVYYCARGGPYGWYFDVWGQGTT |
| | | VTVSS |
| 31C6_HUMZ_VH3 | 146 | EVQLVQSGAEVKKPGASVKVSCKASGYTFSSYVMHWVR |
| (with CDRs underlined) | | QAPGQGLEWIGYIDPYNDGAKYAQKFQGRVTLTRDTST |
| | | STVYMELSSLRSEDTAVYYCARGGPYGWYFDVWGQGTT |
| | | VTVSS |
| 31C6_HUMZ_VH4 | 147 | EVQLVQSGAEVKKPGASVKVSCKASGYTFSSYVMHWVR |
| (with CDRs underlined) | | QAPGQGLEWIGYIDPYNDGAKYAQKFQGRVTLTSDKST |
| | | STVYMELSSLRSEDTAVYYCARGGPYGWYFDVWGQGTT |
| | | VTVSS |
| 31C6_HUMZ_VH5 | 148 | EVQLVQSGAEVKKPGSSVKVSCKASGYTFSSYVMHWVR |
| (with CDRs underlined) | | QAPGQGLEWIGYIDPYNDGAKYAQKFQGRVTLTSDKST |
| | | STAYMELSSLRSEDTAVYYCARGGPYGWYFDVWGQGTT |
| | | VTVSS |
| 31C6_HUMZ_VH6 | 149 | EVQLVQSGAEVKKPGASVKVSCKASGYTFSSYVMHWVR |
| (with CDRs underlined) | | QAPGQGLEWIGYIDPYNDGAKYAQKFQGRVTLTSDKSI |
| | | STAYMELSRLRSDDTVVYYCARGGPYGWYFDVWGQGTT |
| | | VTVSS |
| 31C6_Humz_L1 | 150 | DIQMTQSPSSLSASVGDRVTITCRASEHIYSYLSWYQQ |
| (with CDRs underlined) | | KPGKAPKLLIYNAKTLAEGVPSRFSGSGSGTDFTLTIS |
| | | SLQPEDFATYYCQHHFGSPLTFGQGTRLEIK |
| 31C6_Humz_L2 | 151 | DIQMTQSPSSLSASVGDRVTITCRASEHIYSYLSWYQQ |
| (with CDRs underlined) | | KPGKAPKLLIYNAKTLAEGVPSRFSGSGSGTQFTLTIS |
| | | SLQPEDFATYYCQHHFGSPLTFGQGTRLEIK |
| 31C6_Humz_L3 | 152 | DIQMTQSPSSLSASVGDRVTITCRASEHIYSYLSWYQQ |
| (with CDRs underlined) | | KPGKVPKLLIYNAKTLAEGVPSRFSGSGSGTDFTLTIS |
| | | SLQPEDVATYYCQHHFGSPLTFGQGTRLEIK |
| 31C6_Humz_L4 | 153 | DIQMTQSPSSLSASVGDRVTITCRASEHIYSYLSWYQQ |
| (with CDRs underlined) | | KPGKVPKLLIYNAKTLAEGVPSRFSGSGSGTQFTLTIS |
| | | SLQPEDVATYYCQHHFGSPLTFGQGTRLEIK |
| 31C6 H-CDR2 variant | 154 | YIDPYNDGAKYAQKFQG |
| 31C6 H-CDR2 variant | 155 | YIDPYNDGAKYSQKFQG |
| 18G10-VH sequence | 156 | QVQLMESGPGLVQPSQTLSLTCTVSGFPLTSYTVHWVR |
| | | QPPGKGLEWIGAIWSSGSTDYNSALKSRLNINRDSSKS |
| | | QVFLKMNSLQTEDTAIYFCTKSGWAFFDYWGQGVMVTV |
| | | SS |
| 18G10-VL sequence | 157 | DIQMTQSPSLLSASVGDRVTLNCIASQNIYKSLAWYQL |
| | | KLGEAPKLLIYNANSLQAGIPSRFSGSGSGTDFALTIS |
| | | GLQPEDVATYFCQQYSGGYTFGAGTKLELK |
| 11A11-VH sequence | 158 | EVQLVESGGDLVQPGRSLKISCVASGFTFSDYYMAWVR |
| | | LAPQKGLEWVASISYEGSRTHYGDSVRGRFIISRDNPK |
| | | NILYLQMNSLGSEDTATYFCARHTGTLDWLVYWGQGTL |
| | | VIVSS |
| 11A11-VL sequence | 159 | NIVMAQSPKSMSISAGDRVTMNCKASQNVDNNIAWYQQ |
| | | KPGQSPKLLIFYASNRYSGVPDRFTGGGYGTDFTLTIK |
| | | SVQAEDAAFYYCQRIYNFPTFGSGTKLEIK |
| 14A6 H-CDR3 | 160 | MPSFITLASLSTXEGYFDF |
| CONSENSUS | | X = W, F, Y, I, V, L |
| 14A6 L-CDR2 | 161 | YAX₁X₂LQT |
| CONSENSUS | | X₁ N, S, T, G, D |
| | | X₂ S, N, S, T, Y, Q |
| 14A6 L-CDR3 | 162 | QQYYSGXT |
| CONSENSUS | | X = W, F, Y, I, V, L |
| 14A6 VH | 163 | EVQLQX₁SGX₂GLX₃KPX₄X₅X₆LSLTCX₇VX₈GX₃₀SIX₃₁ |
| PARENTAL | | SDYWGWX₉RX₁₀X₁₁PGX₁₂X₁₃X₁₄EWX₁₅GFITYSGSTSY |
| CONSENSUS | | NPSLKSRX₁₆X₁₇IX₁₈X₁₉DTSKNQFX₂₀LX₂₁LX₂₂SVT |
| | | X₂₃X₂₄DT |
| | | AX₂₅YX₂₆CARMPSFITLASLSTX₂₇EGYFDFWGX₃₂GT |
| | | X₂₈X₂₉TVSS |
| | | X₁ = E or Q |
| | | X₂ = P or A |
| | | X₃ = V or L |
| | | X₄ = S or P |
| | | X₅ = Q or E or G |
| | | X₆ = S or T |
| | | X₇ = S or T or A |
| | | X₈ = T or S |
| | | X₉ = I or V |
| | | X₁₀ = K or Q |
| | | X₁₁ = F or P |
| | | X₁₂ = N or K |
| | | X₁₃ = K or G |
| | | X₁₄ = M or L |
| | | X₁₅ = M or I |
| | | X₁₆ = I or V |
| | | X₁₇ = S or T |
| | | X₁₈ = T or S |
| | | X₁₉ = R or V |
| | | X20 = F or S |
| | | X21 = Q or K |
| | | X22 = H or S |
| | | X23 = T or A |
| | | X24 = D or A |
| | | X25 = T or V |
| | | X26 = S or Y, |
| | | X27 = W, F, Y, I, V or L |
| | | X28 = M, V, L, A, R, N, P, Q, E, G, I, |
| | | H, K, F, S, T, W or Y |
| | | X29 = V, T or L |
| | | X₃₀ = S or G or Y |
| | | X₃₁ = A or S |
| | | X₃₁ = P or Q |
| 14A6 VH HUMANIZED | 164 | EVQLQX₁SGX₂GLX₃KPX₄X₅TLSLTCX₆VX₇GX₈SIX₉SD |
| CONSENSUS | | YWGWX₁₀RX₁₁X₁₂PGKX₁₃X₁₄EWX₁₅GFITYSGSTSYNPS |
| | | LKSRX₁₆TISX₁₇DTSKNQFSLKLX₁₈SVTAX₁₉DTAVYYC |
| | | ARMPSFITLASLSTX₂₀EGYFDFWGQGTX₂₁X₂₂TVSS |
| | | X₁ = E or Q |
| | | X₂ = P or A |
| | | X₃ = V or L |
| | | X₄ = S or P |
| | | X₅ = E or G |
| | | X₆ = T or A or S |
| | | X₇ = S or T |
| | | X₈ = G or S or Y |
| | | X₉ = S or A |
| | | X₁₀ = I or V |
| | | X₁₁ = Q or K |
| | | X₁₂ = P or F |
| | | X₁₃ = G or K |
| | | X₁₄ = L or M |
| | | X₁₅ = I or M |
| | | X₁₆ = V or I |
| | | X₁₇ = V or R |
| | | X₁₈ = S or H |
| | | X₁₉ = A or D |
| | | X₂₀ = W, F, Y, I, V, L |
| | | X₂₁ = M, V, L, A, R, N, P, Q, E, G, I, |
| | | H, K, F, S, T, W or Y |
| | | X₂₂ = V, T or L |
| 14A6 VL | 165 | DIQMTQSPSX₁LSASVGDRVTX₂X₃CKASQSIHKNLAWY |
| PARENTAL | | QQKX₄GX₅X₁₅PKX₆LIYYAX₇X₈LQTGX₉PSRFSGSGSGT |
| CONSENSUS | | DFTLTISX₁₀LQPEDX₁₁ATYX₁₂CQQYYSGX₁₃TFGGGTK |
| | | VEX₁₄K |
| | | X₁ = L or S |
| | | X₂ = L or I |
| | | X₃ = N or T |
| | | X₄ = L or P |
| | | X₅ = E or K |
| | | X₆ = F or L |
| | | X₇ = N, S, T, G or D |
| | | X₈ = S, N, T, Y or Q |
| | | X₉ = I or V |
| | | X₁₀ = G or S |
| | | X₁₁ = V or F |
| | | X₁₂ = F or Y |
| | | X₁₃ = W, F, Y, I, V or L |
| | | X₁₄ = L or I |
| | | X₁₅ = A or V |
| 14A6 VL HUMANIZED | 166 | DIQMTQSPSSLSASVGDRVTITCKASQSIHKNLAWYQQ |
| CONSENSUS | | KPGKX₆PKX₁LIYYAX₂X₃LQTGX₄PSRFSGSGSGTDFTL |
| | | TISSLQPEDX₇ATYYCQQYYSGX₅TFGGGTKVEIK |
| | | X₁ = L or F |
| | | X₂ = N, S, T, G or D |
| | | X₃ = S, N, T, Y or Q |
| | | X₄ = V or I |
| | | X₅ = W, F, Y, I, V or L |
| | | X₆ = A or V |
| | | X₇ = F or V |
| 31C6 H-CDR2 | 167 | YIDPYNX₁X₂AKYX₃X₄KFX₅G |
| CONSENSUS | | X₁ = D, R, L, K, F, S, Y or V |
| | | X₂ = G, R, N, Q, E, L K, S, Y or V |
| | | X₃ = N, A or S |
| | | X₄ = E or Q |
| | | X₅ = K or Q |
| 31C6 L-CDR2 | 168 | X₁X₂KTLAE |
| CONSENSUS | | X₁ N, A, V, W, S, T, R, H G, I or V |
| | | X₂ A, N, I, L, T or V |
| 31C6 VH | 169 | EVQLX₁QSGX₂EX₃X₄KPGX₅SVKX₆SCKASGYTFSSYVM |
| PARENTAL | | HWVX₇QX₈PGQX₉LEWIGYIDPYNX₁₀X₁₁AKYX₁₂X₁₃KF |
| CONSENSUS | | X₁₄GX₁₅X₁₆TLTX₁₇DX₁₈SX₁₉STX₂₀YMELSX₂₁LX₂₂SX₂₃ |
| | | DX₂₄X₂₅VYYCARGGPYGX₂₆YFDVWGX₂₇GTTVTVSS |
| | | X₁ = Q or V |
| | | X₂ = P or A |
| | | X₃ = V or L |
| | | X₄ = V or K |
| | | X₅ = S or A |
| | | X₆ = M or V |
| | | X₇ = K or R |
| | | X₈ = K or A |
| | | X₉ = G or R |
| | | X₁₀ = D, R, L, K, F, S, Y or V |
| | | X₁₁ = G, R, N, Q, E, L K, S, Y or V |
| | | X₁₂ = N, A or S |
| | | X₁₃ = E or Q |
| | | X₁₄ = K or Q |
| | | X₁₅ = R or K |
| | | X₁₆ = A or V |
| | | X₁₇ = S or R |
| | | X₁₈ = K or T |
| | | X₁₉ = S, I, A or T |
| | | X₂₀ = A or V |
| | | X₂₁ = R or S |
| | | X₂₂ = T or R |
| | | X₂₃ = D or E |
| | | X₂₄ = S or T |
| | | X₂₅ = A or V |
| | | X₂₆ = W, A, D, E, F, G, I, K, N, Q, R, |
| | | S, T, V or Y |
| | | X₂₇ = A or Q |
| 31C6 VH | 170 | EVQLVQSGAEVKKPGX₁SVKVSCKASGYTFSSYVMHWV |
| HUMANIZED | | RQAPGQX₂LEWIGYIDPYNX₃X₄AKYX₅X₅KFX₇GRVTLT |
| CONSENSUS | | X₈DX₉SX₁₀STXIIYMELSX₁₂LRSX₁₃DTX₁₄VYYCARGG |
| | | PYGX₁₅YFDVWGQGTTVTVSS |
| | | X₁ = A or S |
| | | X₂ = R or G |
| | | X₃ = D, R, L, K, F, S, Y or V |
| | | X₄ = G, R, N, Q, E, L K, S, Y or V |
| | | X₅ = N, A or S |
| | | X₆ = E or Q |
| | | X₇ = K or Q |
| | | X₈ = R or S |
| | | X₉ = T or K |
| | | X₁₀ = A, T or I |
| | | X₁₁ = A or V |
| | | X₁₂ = S or R |
| | | X₁₃ = E or D |
| | | X₁₄ = A or V |
| | | X₁₅ = W, A, D, E, F, G, I, K, N, Q, R, |
| | | S, T, V or Y |
| 31C6 VL | 171 | DIQMTQSPX₁SLSASVGX₂X₃VTITCRASEHIYSYLSWY |
| PARENTAL | | QQKX₄GKX₅PX₆LLX₇YX₈X₉KTLAEGVPSRFSGSGSGT |
| CONSENSUS | | X₁₀FX₁₁LX₁₂IX₁₃SLQPEDX₁₄X₁₅TYYCQHHFGSPLTFG |
| | | X₁₆GTX₁₇LEX₁₈K |
| | | X₁ = A or S |
| | | X₂ = E or D |
| | | X₃ = T or R |
| | | X₄ = Q or P |
| | | X₅ = S, A or V |
| | | X₆ = Q or K |
| | | X₇ = V or I |
| | | X₈ = N, A, Y, W, S, T, I or V |
| | | X₉ = A, N, I, L, T or V |
| | | X₁₀ = Q or D |
| | | X₁₁ = S or T |
| | | X₁₂ = K or T |
| | | X₁₃ = N or S |
| | | X₁₄ = F or V |
| | | X₁₅ = G or A |
| | | X₁₆ = A or Q |
| | | X₁₇ = T or R |
| | | X₁₈ = L or I |
| 31C6 L-VL | 172 | DIQMTQSPSSLSASVGDRVTITCRASEHIYSYLSWYQQ |
| HUMANIZED | | KPGKX₁PKLLIYX₂X₃KTLAEGVPSRFSGSGSGTX₄FTL |
| CONSENSUS | | TISSLQPEDX₅ATYYCQHHFGSPLTFGQGTRLEIK |
| | | X₁ = A or V |
| | | X₂ = N, A, Y, W, S, T, I or V |
| | | X₃ = A, N, I, L, T or V |
| | | X₄ = D or Q |
| | | X₅ = F or V |
| 31C6 H-CDR3 | 173 | GGPYGXYFDV |
| CONSENSUS | | X₁₅ W, A, D, E, F, G, I, K, N, Q, |
| | | R, S, T, V or Y |
| 31C6 H-CDR3 | 174 | GGPYGAYFDV |
| VARIANT | | |
| 31C6 H-CDR3 | 175 | GGPYGDYFDV |
| VARIANT | | |
| 31C6 H-CDR3 | 176 | GGPYGEYFDV |
| VARIANT | | |
| 31C6 H-CDR3 | 177 | GGPYGFYFDV |
| VARIANT | | |
| 31C6 H-CDR3 | 178 | GGPYGGYFDV |
| VARIANT | | |
| 31C6 H-CDR3 | 179 | GGPYGIYFDV |
| VARIANT | | |
| 31C6 H-CDR3 | 180 | GGPYGKYFDV |
| VARIANT | | |
| 31C6 H-CDR3 | 181 | GGPYGNYFDV |
| VARIANT | | |
| 31C6 H-CDR3 | 182 | GGPYGQYFDV |
| VARIANT | 183 | GGPYGRYFDV |
| 31C6 H-CDR3 | 184 | GGPYGSYFDV |
| VARIANT | 185 | GGPYGTYFDV |
| 31C6 H-CDR3 | 186 | GGPYGVYFDV |
| VARIANT | 187 | GGPYGYYFDV |

In some embodiments, an anti-TIGIT antibody or antigen binding fragment thereof comprises three light chain CDRs of CDRL1, CDRL2, and CDRL3 and/or three heavy chain CDRs of CDRH1, CDRH2, and CDRH3.

In one embodiment, the anti-TIGIT antibody or antigen binding fragment thereof comprises a CDRH1 comprising SEQ ID NO:35, a CDRH2 comprising SEQ ID NO:36, a CDRH3 comprising any of SEQ ID NOs:37, 103, 104, 105, 106, 107, or 160, a CDRL1 comprising SEQ ID NO: 38, a CDRL2 comprising any of SEQ ID NOs:39, 89, 90, 91, 92, 93, 94, 95, 96, 97, or 69, and a CDRL3 comprising any of SEQ ID NOs:40, 98, 99, 100, 101, 102, or 162.

In another embodiment, the anti-TIGIT antibody or antigen binding fragment thereof comprises a CDRH1 comprising SEQ ID NO:81, a CDRH2 comprising SEQ ID NO:82, a CDRH3 comprising SEQ ID NO:83, a CDRL1 comprising SEQ ID NO:84, a CDRL2 comprising SEQ ID NO:85, and a CDRL3 comprising SEQ ID NO:86.

In another embodiment, the anti-TIGIT antibody or antigen binding fragment thereof comprises a CDRH1 comprising SEQ ID NO:108, a CDRH2 comprising any of SEQ ID NOs:109, 116, 117, 118, 119, 120, 121, 122, 123, 124, 125, 126, 127, 128, 129, 130, 131, 154, 155 or 167, a CDRH3 comprising any of SEQ ID NOs:110, 173, 174, 175, 176, 177, 178, 179, 180, 181, 182, 183, 184, 185, 186 or 187, a CDRL1 comprising SEQ ID NO:111, a CDRL2 comprising any of SEQ ID NOs:112, 132, 133, 134, 135, 136, 137, 138, 139, 140, 141, 142 or 168, and a CDRL3 comprising the amino acid sequence of SEQ ID NO:113.

In one embodiment, the anti-TIGIT antibody or antigen binding fragment thereof comprises a CDRH1 comprising SEQ ID NO:35, a CDRH2 comprising of SEQ ID NO:36, a CDRH3 comprising SEQ ID NO:37, a CDRL1 comprising SEQ ID NO:38, a CDRL2 comprising SEQ ID NO:39, and a CDRL3 comprising the amino acid sequence of SEQ ID NO:40.

In one embodiment, the anti-TIGIT antibody or antigen binding fragment thereof comprises CDRH1 comprising SEQ ID NO:108, a CDRH2 comprising any one of SEQ ID NO:109, 154 or 145, a CDRH3 comprising SEQ ID NO:110, a CDRL1 comprising SEQ ID NO:111, a CDRL2 comprising SEQ ID NO:112, and a CDRL3 comprising the amino acid sequence of SEQ ID NO:113.

In another embodiment, the anti-TIGIT antibody or antigen binding fragment thereof comprises a CDRH1 comprising the amino acid sequence of SEQ ID NO:108, a CDRH2 comprising SEQ ID NO: 154, a CDRH3 comprising SEQ ID NO:110, a CDRL1 comprising SEQ ID NO:111, a CDRL2 comprising SEQ ID NO:112, and a CDRL3 comprising SEQ ID NO:113.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising the SEQ ID NO:41 and a variable light chain region comprising SEQ ID NO:42.

In one embodiment, the anti-TIGIT antibody or antigen binding fragment thereof comprises a variable heavy chain region comprising SEQ ID NO:87 and a variable light chain region comprising SEQ ID NO:88.

In one embodiment, the anti-TIGIT antibody or antigen binding fragment thereof comprises a variable heavy chain region comprising SEQ ID NO: 114 and a variable light chain region comprising SEQ ID NO:115.

In one embodiment, the anti-TIGIT antibody or antigen binding fragment thereof comprises a variable heavy chain region comprising any of SEQ ID NOs: 43-58, 65-75 and 87 and a variable light chain region comprising of any one of SEQ ID NOs: 59-64, 76-80 and 88.

In one embodiment, the anti-TIGIT antibody or antigen binding fragment thereof comprises a variable heavy chain region comprising any of SEQ ID NOs: 144-149 and a variable light chain region comprising any of SEQ ID NOs: 150-153.

In one embodiment, the anti-TIGIT antibody or antigen binding fragment thereof comprises a variable heavy chain region comprising a variable heavy chain region comprising SEQ ID NO:148 and a variable light chain region comprising SEQ ID NO:152.

In one embodiment, the anti-TIGIT antibody or antigen binding fragment thereof comprises a variable heavy chain region comprising SEQ ID NO:147 and a variable light chain region comprising SEQ ID NO:150.

In one embodiment, the anti-TIGIT antibody or antigen binding fragment thereof comprises a variable heavy chain region comprising SEQ ID NO:148 and a variable light chain region comprising SEQ ID NO:153.

In one embodiment, the anti-TIGIT antibody or antigen binding fragment thereof comprises a variable heavy chain region comprising SEQ ID NO:163 and a variable light chain region comprising SEQ ID NO:165.

In one embodiment, the anti-TIGIT antibody or antigen binding fragment thereof comprises a variable heavy chain region comprising SEQ ID NO:169 and a variable light chain region comprising SEQ ID NO:171.

In one embodiment, the anti-TIGIT antibody or antigen binding fragment thereof comprises a variable heavy chain region comprising SEQ ID NO:164 and a variable light chain region comprising SEQ ID NO:166.

In one embodiment, the anti-TIGIT antibody or antigen binding fragment thereof comprises a variable heavy chain region comprising SEQ ID NO:170 and a variable light chain region comprising SEQ ID NO:172.

TABLE 5
Exemplary sequences of anti-TIGIT antibodies

| | SEQ ID | |
|---|---|---|
| Description | NO | SEQUENCE |
| 14D7 H- | 188 | GAWMD |
| CDR1 | | |
| 14D7 H- | 189 | EIRTKVNNHATNYGESVKG |
| CDR2 | | |
| 14D7 H- | 190 | ALYDGFYFDY |
| CDR3 | | |
| 14D7 L- | 191 | SASSSVSSGYLY |
| CDR1 | | |
| 14D7 L- | 192 | GTSTLAS |
| CDR2 | | |
| 14D7 L- | 193 | HQWSSFPYT |
| CDR3 | | |
| 14D7 VH | 194 | EVKLEESGGGLVQPGGSMKLSCVASGFTFSGAWMDWVRQSP |
| PARENTAL | | EKGLEWVAEIRTKVNNHATNYGESVKGRFTISRDDSKSSVYLQ |
| | | MNNLRAEDSGIYYCRGALYDGFYFDYWGQGTTLTVSS |
| 14D7 VL | 195 | QIVLTQSPAIMSASPGEKVNLTCSASSSVSSGYLYWYQQKPGSS |
| PARENTAL | | PKLWIYGTSTLASGVPARFSGSGSGTSYSLTISNMEAEDAASYF |
| | | CHQWSSFPYTFGGGTKLEMK |
| Hu14D7 VH | 196 | EVQLVESGGGLVQPGGSLKLSCAASGFTFSGAX₁X₂DWVRQAP |
| humanized | | GKGLEWVAEIRTKVNNHATNYGESVKGRFTISRDX₃SKX₄X₅V |
| consensus | | YLQX₆X₇X₈LRAEDX₉AVYYCRGALYX₁₀X₁₁FYFDYWGQGTLVT |
| sequence | | VSS |
| | | X₁ = W, A, R, N, D, Q, E, G, H, I, L, K, F, P, S, T, Y, V |
| | | X₂ = M, V, L, I, G, A, S, T |
| | | X₃ = D, A, R, N, Q, E, G, H, I, L, K, F, S, T, Y, V |
| | | X₄ = S, N |
| | | X₅ = T, S |
| | | X₆ = M, L |
| | | X₇ = N, S |
| | | X₈ = S, N |
| | | X₉ = T, S |
| | | X₁₀ = D, A, R, N, Q, E, G, H, I, L, K, F, P, S, T, W, Y, V |
| | | X₁₁ = G, A, R, N, D, Q, E, H, I, L, K, F, P, S, T, W, Y, V |
| Hu14D7 VH1 | 197 | EVQLVESGGGLVQPGGSLKLSCAASGFTFSGAWMDWVRQAP |
| (Humanized | | GKGLEWVAEIRTKVNNHATNYGESVKGRFTISRDDSKSTVYL |
| VH chain) | | QMNSLRAEDTAVYYCRGALYDGFYFDYWGQGTLVTVSS |
| Hu14D7 VH2 | 198 | EVQLVESGGGLVQPGGSLKLSCAASGFTFSGAWMDWVRQAP |
| (Humanized | | GKGLEWVAEIRTKVNNHATNYGESVKGRFTISRDDSKSSVYL |
| VH chain) | | QMNSLRAEDTAVYYCRGALYDGFYFDYWGQGTLVTVSS |
| Hu14D7 VH3 | 199 | EVQLVESGGGLVQPGGSLKLSCAASGFTFSGAWMDWVRQAP |
| (Humanized | | GKGLEWVAEIRTKVNNHATNYGESVKGRFTISRDDSKNTVYL |
| VH chain) | | QMNSLRAEDTAVYYCRGALYDGFYFDYWGQGTLVTVSS |
| Hu14D7 VL | 200 | EIVLTQSPATLSLSPGERAX₁LSCSASSSVSSGYLYWYQQKPGQ |
| humanized | | APX₇LX₂IYGTSTLASGX₈PARFSGSGSGTDYTLTISSX₃EPEDX₄A |
| consensus | | VYYCHQX₅SSFPYTFGQGTKLEX₆K |
| Sequence | | X₁ = T, S |
| | | X₂ = W, A, R, N, D, Q, E, G, H, I, L, K, F, P, S, T, Y, V |
| | | X₃ = L, V, I |
| | | X₄ = F, V, L, I, T |
| | | X₅ = W, A, R, N, D, Q, E, G, H, I, L, K, F, P, S, T, Y, V |
| | | X₆ = I, L |
| | | X₇ = K, R |
| | | X₈ = V, I |
| Hu14D7V L1 | 201 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQWSSFPYTFGQGTKLEIK |
| Hu14D7V L2 | 202 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQWSSFPYTFGQGTKLEIK |
| Hu14D7V L3 | 203 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQWSSFPYTFGQGTKLEIK |
| 26B10 H- | 204 | EFTMH |
| CDR1 | | |
| 26B10 H- | 205 | GLKPDNGGISYNQKFKG |
| CDR2 | | |
| 26B10 H- | 206 | GAYYRYDADY |
| CDR3 | | |
| 26B10 L- | 207 | KASQDVKTAVA |
| CDR1 | | |
| 26B10 L- | 208 | SASYRNT |
| CDR2 | | |
| 26B10 L- | 209 | QQHYSTPFT |
| CDR3 | | |
| 26B10 VH | 210 | EVQLQQSGPELVKPGASVKISCKTSGYTFTEFTMHWVKQSHG |
| PARENTAL | | KSLEWIGGLKPDNGGISYNQKFKGRATLAVDKSSNTAYMELR |
| | | SLTSEDSAVYYCARGAYYRYDADYWGQGTTLTVSS |
| 26B10 VL | 211 | DIVLTQSHKFMSTSVGDRVSITCKASQDVKTAVAWYQQKSGQ |
| PARENTAL | | SPKLLIYSASYRNTGVPDRFTGSGSGTDFTFTIDSVQAEDLAVY |
| | | FCQQHYSTPFTFGTGTKLELK |
| 26B10 VH | 212 | EVQLVQSGAEVKKPGASVKISCKX₁SGYTFTEFTX₂HWVX₃QAP |
| HUMANIZED | | GKGLEWIGGLKPDX₄X₅GISYNQKFKGRATLTVDX₆STX₇TAYX₈ |
| CONSESUS | | ELSSLRSEDX₉AVYYCARGAYYRYX₁₀X₁₁DYWGQGTLVTVSS |
| Sequence | | X₁ = T, V |
| | | X₂ = M, V, L, I, G, A, S, T |
| | | X₃ = K, R |
| | | X₄ = N, A, R, D, Q, E, G, H, I, L, K, F, P, S, T, W, Y, V |
| | | X₅ = G, A, R, N, D, Q, E, H, I, L, K, F, P, S, T, W, Y, V |
| | | X₆ = k, t, d, s |
| | | X₇ = N, S |
| | | X₈ = M, V, L, I, G, A, S, T |
| | | X₉ = T, S |
| | | X₁₀ = D, A, R, N, Q, E, G, H, I, L, K, F, P, S, T, W, Y, V |
| | | X₁₁ = A, R, N, D, Q, E, G, H, I, L, K, F, P, S, T, W, Y, V, M |
| Hu26B10 VH1 | 213 | EVQLVQSGAEVKKPGASVKISCKTSGYTFTEFTMHWVKQAPG |
| (Humanized | | KGLEWIGGLKPDNGGISYNQKFKGRATLTVDKSTNTAYMELS |
| VH chain) | | SLRSEDTAVYYCARGAYYRYDADYWGQGTLVTVSS |
| Hu26B10 VH2 | 214 | EVQLVQSGAEVKKPGASVKISCKTSGYTFTEFTMHWVRQAPG |
| (Humanized | | KGLEWIGGLKPDNGGISYNQKFKGRATLTVDKSTSTAYMELSS |
| VH chain) | | LRSEDTAVYYCARGAYYRYDADYWGQGTLVTVSS |
| Hu26B10 VH3 | 215 | EVQLVQSGAEVKKPGASVKISCKVSGYTFTEFTMHWVRQAPG |
| (Humanized | | KGLEWIGGLKPDNGGISYNQKFKGRATLTVDTSTSTAYMELSS |
| VH chain) | | LRSEDTAVYYCARGAYYRYDADYWGQGTLVTVSS |
| 26B10 VL | 216 | DIQLTQSPSSLSASVGDRVTITCKASQDVKTAVAWYQQKPGKA |
| HUMANIZED | | PKLLIYSASYRX₁X₂GVP |
| CONSESUS | | X₃RFSGSGSGTDFTX₄TISSLQPEDFATYYCQQHYSTPFTFGQGT |
| sequence | | KLEIK |
| | | X₁ = N, Q, D, E |
| | | X₂ = T, S, A |
| | | X₃ = D, S |
| | | X₄ = F, L |
| Hu26B10 VL1 | 217 | DIQLTQSPSSLSASVGDRVTITCKASQDVKTAVAWYQQKPGKA |
| (Humanized | | PKLLIYSASYRNTGVPDRFSGSGSGTDFTFTISSLQPEDFATYYC |
| VL chain) | | QQHYSTPFTFGQGTKLEIK |
| Hu26B10 VL2 | 218 | DIQLTQSPSSLSASVGDRVTITCKASQDVKTAVAWYQQKPGKA |
| (Humanized | | PKLLIYSASYRNTGVPSRFSGSGSGTDFTFTISSLQPEDFATYYC |
| VL chain) | | QQHYSTPFTFGQGTKLEIK |
| Hu26B10 VL3 | 219 | DIQLTQSPSSLSASVGDRVTITCKASQDVKTAVAWYQQKPGKA |
| (Humanized | | PKLLIYSASYRNTGVPSRFSGSGSGTDFTLTISSLQPEDFATYYC |
| VL chain) | | QQHYSTPFTFGQGTKLEIK |
| 14D7 H- | 220 | ALYEGFYFDY |
| CDR3(D104E) | | |
| 14D7 H- | 221 | ALYDAFYFDY |
| CDR3 | | |
| (G105A) | | |
| 14D7 H- | 222 | ALYDSFYFDY |
| CDR3(G105S) | | |
| Hu14D7 VH1 | 223 | EVQLVESGGGLVQPGGSLKLSCAASGFTFSGAWMDWVRQAP |
| (Humanized | | GKGLEWVAEIRTKVNNHATNYGESVKGRFTISRDDSKSTVYL |
| VH chain) | | QMNSLRAEDTAVYYCRGALYEGFYFDYWGQGTLVTVSS |
| (D104E) | | |
| Hu14D7 VH1 | 224 | EVQLVESGGGLVQPGGSLKLSCAASGFTFSGAWMDWVRQAP |
| (Humanized | | GKGLEWVAEIRTKVNNHATNYGESVKGRFTISRDDSKSTVYL |
| VH chain) | | QMNSLRAEDTAVYYCRGALYDAFYFDYWGQGTLVTVSS |
| (G105A) | | |
| Hu14D7 VH1 | 225 | EVQLVESGGGLVQPGGSLKLSCAASGFTFSGAWMDWVRQAP |
| (Humanized | | GKGLEWVAEIRTKVNNHATNYGESVKGRFTISRDDSKSTVYL |
| VH chain) | | QMNSLRAEDTAVYYCRGALYDSFYFDYWGQGTLVTVSS |
| (G105S) | | |
| Hu14D7 VH2 | 226 | EVQLVESGGGLVQPGGSLKLSCAASGFTFSGAWMDWVRQAP |
| (Humanized | | GKGLEWVAEIRTKVNNHATNYGESVKGRFTISRDDSKSSVYL |
| VH chain) | | QMNSLRAEDTAVYYCRGALYDEFYFDYWGQGTLVTVSS |
| (D104E) | | |
| Hu14D7 VH2 | 227 | EVQLVESGGGLVQPGGSLKLSCAASGFTFSGAWMDWVRQAP |
| (Humanized | | GKGLEWVAEIRTKVNNHATNYGESVKGRFTISRDDSKSSVYL |
| VH chain) | | QMNSLRAEDTAVYYCRGALYDAFYFDYWGQGTLVTVSS |
| (G105A) | | |
| Hu14D7 VH2 | 228 | EVQLVESGGGLVQPGGSLKLSCAASGFTFSGAWMDWVRQAP |
| (Humanized | | GKGLEWVAEIRTKVNNHATNYGESVKGRFTISRDDSKSSVYL |
| VH chain) | | QMNSLRAEDTAVYYCRGALYDSFYFDYWGQGTLVTVSS |
| (G105S) | | |
| Hu14D7 VH3 | 229 | VQLVESGGGLVQPGGSLKLSCAASGFTFSGAWMDWVRQAP |
| (Humanized | | GKGLEWVAEIRTKVNNHATNYGESVKGRFTISRDDSKNTVYL |
| VH chain) | | QMNSLRAEDTAVYYCRGALYEGFYFDYWGQGTLVTVSS |
| (D104E) | | |
| Hu14D7 VH3 | 230 | EVQLVESGGGLVQPGGSLKLSCAASGFTFSGAWMDWVRQAP |
| (Humanized | | GKGLEWVAEIRTKVNNHATNYGESVKGRFTISRDDSKNTVYL |
| VH chain) | | QMNSLRAEDTAVYYCRGALYDAFYFDYWGQGTLVTVSS |
| (G105A) | | |
| Hu14D7 VH3 | 231 | EVQLVESGGGLVQPGGSLKLSCAASGFTFSGAWMDWVRQAP |
| (Humanized | | GKGLEWVAEIRTKVNNHATNYGESVKGRFTISRDDSKNTVYL |
| VH chain) | | QMNSLRAEDTAVYYCRGALYDSFYFDYWGQGTLVTVSS |
| (G105S) | | |
| 14D7 L- | 232 | HQASSFPYT |
| CDR3 | | |
| (W92A) | | |
| 14D7 L- | 233 | HQDSSFPYT |
| CDR3(W92D) | | |
| 14D7 L- | 234 | HQESSFPYT |
| CDR3(W92E) | | |
| 14D7 L- | 235 | HQFSSFPYT |
| CDR3(W92F) | | |
| 14D7 L- | 236 | HQGSSFPYT |
| CDR3(W92G) | | |
| 14D7 L - | 237 | HQHSSFPYT |
| CDR3 | | |
| (W92H) | | |
| Hu14D7V L1 | 238 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQASSFPYTFGQGTKLEIK |
| (W92A) | | |
| Hu14D7V L1 | 239 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQDSSFPYTFGQGTKLEIK |
| (W92D) | | |
| Hu14D7V L1 | 240 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQESSFPYTFGQGTKLEIK |
| (W92E) | | |
| Hu14D7V L1 | 241 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQFSSFPYTFGQGTKLEIK |
| (W92F) | | |
| Hu14D7V L1 | 242 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQGSSFPYTFGQGTKLEIK |
| (W92G) | | |
| Hu14D7V L1 | 243 | EIVLTQSPATLSLSPGERATLSCSASSSVSSGYLYWYQQKPGQA |
| (Humanized | | PKLWIYGTSTLASGVPARFSGSGSGTDYTLTISSLEPEDFAVYY |
| VL chain) | | CHQHSSFPYTFGQGTKLEIK |
| (W92H) | | |
| Hu14D7V L2 | 244 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQASSFPYTFGQGTKLEIK |
| (W92A) | | |
| Hu14D7V L2 | 245 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQDSSFPYTFGQGTKLEIK |
| (W92D) | | |
| Hu14D7V L2 | 246 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQESSFPYTFGQGTKLEIK |
| (W92E) | | |
| Hu14D7V L2 | 247 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQFSSFPYTFGQGTKLEIK |
| (W92F) | | |
| Hu14D7V L2 | 248 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQGSSFPYTFGQGTKLEIK |
| (W92G) | | |
| Hu14D7V L2 | 249 | EIVLTQSPATLSLSPGERATLSCSASSSVSSGYLYWYQQKPGQA |
| (Humanized | | PRLWIYGTSTLASGVPARFSGSGSGTDYTLTISSLEPEDFAVYY |
| VL chain) | | CHQHSSFPYTFGQGTKLEIK |
| (W92H) | | |
| Hu14D7V L3 | 250 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQASSFPYTFGQGTKLEIK |
| (W92A) | | |
| Hu14D7V L3 | 251 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQDSSFPYTFGQGTKLEIK |
| (W92D) | | |
| Hu14D7V L3 | 252 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQESSFPYTFGQGTKLEIK |
| (W92E) | | |
| Hu14D7V L3 | 253 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQFSSFPYTFGQGTKLEIK |
| (W92H) | | |
| Hu14D7V L3 | 254 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQGSSFPYTFGQGTKLEIK |
| (W92G) | | |
| Hu14D7V L3 | 255 | EIVLTQSPATLSLSPGERATLSCSASSSVSSGYLYWYQQKPGQA |
| (Humanized | | PRLWIYGTSTLASGIPARFSGSGSGTDYTLTISSLEPEDFAVYYC |
| VL chain) | | HQHSSFPYTFGQGTKLEIK |
| (W92H) | | |
| 26B10 H- | 256 | GLKPDQGGISYNQKFKG |
| CDR2 (N55Q) | | |
| 26B10 H- | 257 | GLKPDDGGISYNQKFKG |
| CDR2(N55D) | | |
| 26B10 H- | 258 | GLKPDNAGISYNQKFKG |
| CDR2(N56A) | | |
| 26B10 H- | 259 | GLKPDTGGISYNQKFKG |
| CDR2(N55T) | | |
| 26B10 H- | 260 | GLKPDSGGISYNQKFKG |
| CDR2(N55S) | | |
| 26B10 H- | 261 | GLKPDGGGISYNQKFKG |
| CDR2(N55G) | | |
| 26B10 H- | 262 | GLKPDNSGISYNQKFKG |
| CDR2(G56S) | | |
| 26B10 H- | 263 | GLKPDNTGISYNQKFKG |
| CDR2(G56T) | | |
| Hu26B10 VH1 | 264 | EVQLVQSGAEVKKPGASVKISCKTSGYTFTEFTMHWVKQAPG |
| (Humanized | | KGLEWIGGLKPDQGGISYNQKFKGRATLTVDKSTNTAYMELS |
| VH | | SLRSEDTAVYYCARGAYYRYDADYWGQGTLVTVSS |
| chain)(N55Q) | | |
| Hu26B10 VH1 | 265 | EVQLVQSGAEVKKPGASVKISCKTSGYTFTEFTMHWVKQAPG |
| (Humanized | | KGLEWIGGLKPDDGGISYNQKFKGRATLTVDKSTNTAYMELS |
| VH | | SLRSEDTAVYYCARGAYYRYDADYWGQGTLVTVSS |
| chain)(N55D) | | |
| Hu26B10 VH1 | 266 | EVQLVQSGAEVKKPGASVKISCKTSGYTFTEFTMHWVKQAPG |
| (Humanized | | KGLEWIGGLKPDNAGISYNQKFKGRATLTVDKSTNTAYMELS |
| VH | | SLRSEDTAVYYCARGAYYRYDADYWGQGTLVTVSS |
| chain)(G56A) | | |
| Hu26B10 VH1 | 267 | EVQLVQSGAEVKKPGASVKISCKTSGYTFTEFTMHWVKQAPG |
| (Humanized | | KGLEWIGGLKPDTGGISYNQKFKGRATLTVDKSTNTAYMELS |
| VH | | SLRSEDTAVYYCARGAYYRYDADYWGQGTLVTVSS |
| chain)(N55T) | | |
| Hu26B10 VH1 | 268 | EVQLVQSGAEVKKPGASVKISCKTSGYTFTEFTMHWVKQAPG |
| (Humanized | | KGLEWIGGLKPDSGGISYNQKFKGRATLTVDKSTNTAYMELSS |
| VH | | LRSEDTAVYYCARGAYYRYDADYWGQGTLVTVSS |
| chain)(N55S) | | |
| Hu26B10 VH1 | 269 | EVQLVQSGAEVKKPGASVKISCKTSGYTFTEFTMHWVKQAPG |
| (Humanized | | KGLEWIGGLKPDGGGISYNQKFKGRATLTVDKSTNTAYMELS |
| VH | | SLRSEDTAVYYCARGAYYRYDADYWGQGTLVTVSS |
| chain)(N55G) | | |
| Hu26B10 VH1 | 270 | EVQLVQSGAEVKKPGASVKISCKTSGYTFTEFTMHWVKQAPG |
| (Humanized | | KGLEWIGGLKPDNSGISYNQKFKGRATLTVDKSTNTAYMELSS |
| VH | | LRSEDTAVYYCARGAYYRYDADYWGQGTLVTVSS |
| chain)(G56S) | | |
| Hu26B10 VH1 | 271 | EVQLVQSGAEVKKPGASVKISCKTSGYTFTEFTMHWVKQAPG |
| (Humanized | | KGLEWIGGLKPDNTGISYNQKFKGRATLTVDKSTNTAYMELS |
| VH | | SLRSEDTAVYYCARGAYYRYDADYWGQGTLVTVSS |
| chain)(G56T) | | |
| Hu26B10 VH2 | 272 | EVQLVQSGAEVKKPGASVKISCKTSGYTFTEFTMHWVRQAPG |
| (Humanized | | KGLEWIGGLKPDQGGISYNQKFKGRATLTVDKSTSTAYMELSS |
| VH | | LRSEDTAVYYCARGAYYRYDADYWGQGTLVTVSS |
| chain)(N55Q) | | |
| Hu26B10 VH2 | 273 | EVQLVQSGAEVKKPGASVKISCKTSGYTFTEFTMHWVRQAPG |
| (Humanized | | KGLEWIGGLKPDDGGISYNQKFKGRATLTVDKSTSTAYMELSS |
| VH | | LRSEDTAVYYCARGAYYRYDADYWGQGTLVTVSS |
| chain)(N55D) | | |
| Hu26B10 VH1 | 274 | EVQLVQSGAEVKKPGASVKISCKTSGYTFTEFTMHWVRQAPG |
| (Humanized | | KGLEWIGGLKPDNAGISYNQKFKGRATLTVDKSTSTAYMELSS |
| VH | | LRSEDTAVYYCARGAYYRYDADYWGQGTLVTVSS |
| chain)(G56A) | | |
| Hu26B10 VH2 | 275 | EVQLVQSGAEVKKPGASVKISCKTSGYTFTEFTMHWVRQAPG |
| (Humanized | | KGLEWIGGLKPDTGGISYNQKFKGRATLTVDKSTSTAYMELSS |
| VH | | LRSEDTAVYYCARGAYYRYDADYWGQGTLVTVSS |
| chain)(N55T) | | |
| Hu26B10 VH2 | 276 | EVQLVQSGAEVKKPGASVKISCKTSGYTFTEFTMHWVRQAPG |
| (Humanized | | KGLEWIGGLKPDSGGISYNQKFKGRATLTVDKSTSTAYMELSS |
| VH | | LRSEDTAVYYCARGAYYRYDADYWGQGTLVTVSS |
| chain)(N55S) | | |
| Hu26B10 VH2 | 277 | EVQLVQSGAEVKKPGASVKISCKTSGYTFTEFTMHWVRQAPG |
| (Humanized | | KGLEWIGGLKPDGGGISYNQKFKGRATLTVDKSTSTAYMELSS |
| VH | | LRSEDTAVYYCARGAYYRYDADYWGQGTLVTVSS |
| chain)(N55G) | | |
| Hu26B10 VH2 | 278 | EVQLVQSGAEVKKPGASVKISCKTSGYTFTEFTMHWVRQAPG |
| (Humanized | | KGLEWIGGLKPDNSGISYNQKFKGRATLTVDKSTSTAYMELSS |
| VH | | LRSEDTAVYYCARGAYYRYDADYWGQGTLVTVSS |
| chain)(G56S) | | |
| Hu26B10 VH2 | 279 | EVQLVQSGAEVKKPGASVKISCKTSGYTFTEFTMHWVRQAPG |
| (Humanized | | KGLEWIGGLKPDNTGISYNQKFKGRATLTVDKSTSTAYMELSS |
| VH | | LRSEDTAVYYCARGAYYRYDADYWGQGTLVTVSS |
| chain)(G56T) | | |
| Hu26B10 VH3 | 280 | EVQLVQSGAEVKKPGASVKISCKVSGYTFTEFTMHWVRQAPG |
| (Humanized | | KGLEWIGGLKPDQGGISYNQKFKGRATLTVDTSTSTAYMELSS |
| VH | | LRSEDTAVYYCARGAYYRYDADYWGQGTLVTVSS |
| chain)(N55Q) | | |
| Hu26B10 VH3 | 281 | EVQLVQSGAEVKKPGASVKISCKVSGYTFTEFTMHWVRQAPG |
| (Humanized | | KGLEWIGGLKPDDGGISYNQKFKGRATLTVDTSTSTAYMELSS |
| VH | | LRSEDTAVYYCARGAYYRYDADYWGQGTLVTVSS |
| chain)(N55D) | | |
| Hu26B10 VH3 | 282 | EVQLVQSGAEVKKPGASVKISCKVSGYTFTEFTMHWVRQAPG |
| (Humanized | | KGLEWIGGLKPDNAGISYNQKFKGRATLTVDTSTSTAYMELSS |
| VH | | LRSEDTAVYYCARGAYYRYDADYWGQGTLVTVSS |
| chain)(G56A) | | |
| Hu26B10 VH3 | 283 | EVQLVQSGAEVKKPGASVKISCKVSGYTFTEFTMHWVRQAPG |
| (Humanized | | KGLEWIGGLKPDTGGISYNQKFKGRATLTVDTSTSTAYMELSS |
| VH | | LRSEDTAVYYCARGAYYRYDADYWGQGTLVTVSS |
| chain)(N55T) | | |
| Hu26B10 VH3 | 284 | EVQLVQSGAEVKKPGASVKISCKVSGYTFTEFTMHWVRQAPG |
| (Humanized | | KGLEWIGGLKPDSGGISYNQKFKGRATLTVDTSTSTAYMELSS |
| VH | | LRSEDTAVYYCARGAYYRYDADYWGQGTLVTVSS |
| chain)(N55S) | | |
| Hu26B10 VH3 | 285 | EVQLVQSGAEVKKPGASVKISCKVSGYTFTEFTMHWVRQAPG |
| (Humanized | | KGLEWIGGLKPDGGGISYNQKFKGRATLTVDTSTSTAYMELSS |
| VH | | LRSEDTAVYYCARGAYYRYDADYWGQGTLVTVSS |
| chain)(N55G) | | |
| Hu26B10 VH3 | 286 | EVQLVQSGAEVKKPGASVKISCKVSGYTFTEFTMHWVRQAPG |
| (Humanized | | KGLEWIGGLKPDNSGISYNQKFKGRATLTVDTSTSTAYMELSS |
| VH | | LRSEDTAVYYCARGAYYRYDADYWGQGTLVTVSS |
| chain)(G56S) | | |
| Hu26B10 VH3 | 287 | EVQLVQSGAEVKKPGASVKISCKVSGYTFTEFTMHWVRQAPG |
| (Humanized | | KGLEWIGGLKPDNTGISYNQKFKGRATLTVDTSTSTAYMELSS |
| VH | | LRSEDTAVYYCARGAYYRYDADYWGQGTLVTVSS |
| chain)(G56T) | | |
| hTIGIT | 288 | SSTTAQVNWEQQDQL |
| epitope(24-41) | | |
| hTIGIT | 289 | IYHTYPDGT |
| epitope(85-93) | | |
| hTIGIT | 290 | GRIFL |
| epitope(96-100) | | |

In one embodiment, the anti-TIGIT antibody or antigen binding fragment comprises a CDRH1 comprising SEQ ID NO:188, a CDRH2 comprising SEQ ID NO: 189, a CDRH3 comprising any of SEQ ID NOs: 190, 220, 221, or 222, a CDRL1 comprising SEQ ID NO: 191, a CDRL2 comprising SEQ ID NO: 192, and a CDRL3 comprising any of SEQ ID NOs:193, 232, 233, 234, 235, 236, or 237.

In one embodiment, the anti-TIGIT antibody or antigen binding fragment thereof comprises a CDRH1 comprising SEQ ID NO:204, a CDRH2 comprising any of SEQ ID NOs: 205, 256, 257, 258, 259, 260, 261, 262, or 263, a CDRH3 comprising SEQ ID NO:206, a CDRL1 comprising SEQ ID NO:207, a CDRL2 comprising SEQ ID NO:208, and a CDRL3 comprising SEQ ID NO:209.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising SEQ ID NO:194 and a variable light chain region comprising SEQ ID NO:195.

In one embodiment, the anti-TIGIT antibody or antigen binding fragment thereof comprises a variable heavy chain region comprising SEQ ID NO:196 and a variable light chain region comprising SEQ ID NO:200.

In one embodiment, the anti-TIGIT antibody or antigen binding fragment thereof comprises a variable heavy chain region comprising SEQ ID NO: 210 and a variable light chain region comprising SEQ ID NO:211.

In one embodiment, the anti-TIGIT antibody or antigen binding fragment thereof comprises a variable heavy chain region comprising SEQ ID NO: 212 and a variable light chain region comprising SEQ ID NO:216.

In one embodiment, the anti-TIGIT antibody or antigen binding fragment thereof comprises a variable heavy chain region comprising any of SEQ ID NOs: 197, 198, 199, 223, 224, 225, 226, 227, 228, 229, 230, and 231 and a variable light chain region comprising any of SEQ ID NOs: 201, 202, 203, 238, 239, 240, 241, 242, 243, 244, 245, 246, 247, 248, 249, 250, 251, 252, 253, 254, and 255.

In one embodiment, the anti-TIGIT antibody or antigen binding fragment thereof comprises a variable heavy chain region comprising any of SEQ ID NOs: 213, 214, 215, 264, 265, 266, 267, 268, 269, 270, 271, 272, 273, 274, 275, 276, 277, 278, 279, 280, 281, 282, 283, 284, 285, and 286 and a variable light chain region comprising any of SEQ ID NOs: 217, 218, and 219.

Additional anti-TIGIT antibodies which may be used in the formulations described herein include those disclosed, for example, in PCT International Application No. WO 2016/106302; WO 2016/011264; and WO 2009/126688.

TABLE 6
Exemplary Heavy Chain Sequences

| | | |
|---|---|---|
| Heavy | 291 | ASTKGPSVFPLAPSSKSTSGGTAALGCLVKDYFPE |
| chain | | PVTVSWNSGALTSGVHTFPAVLQSSGLYSLSSVVT |
| constant | | VPSSSLGTQTYICNVNHKPSNTKVDKKVEPKSCDK |
| domain- | | THTCPPCPAPELLGGPSVFLFPPKPKDTLMISRTP |
| IgG1 | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSRDELTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| Heavy | 292 | TKGPSVFPLAPCSRSTSESTAALGCLVKDYFPEPV |
| chain | | TVSWNSGALTSGVHTFPAVLQSSGLYSLSSVVTVP |
| constant | | SSSLGTKTYTCNVDHKPSNTKVDKRVESKYGPPCP |
| domain- | | PCPAPEFLGGPSVFLFPPKPKDTLMISRTPEVTCV |
| IgG4 | | VVDVSQEDPEVQFNWYVDGVEVHNAKTKPREEQFN |
| S228P | | STYRVVSVLTVLHQDWLNGKEYKCKVSNKGLPSSI |
| | | EKTISKAKGQPREPQVYTLPPSQEEMTKNQVSLTC |
| | | LVKGFYPSDIAVEWESNGQPENNYKTTPPVLDSDG |
| | | SFFLYSRLTVDKSRWQEGNVFSCSVMHEALHNHYT |
| | | QKSLSLSLGK |
| Kappa | 293 | VAAPSVFIFPPSDEQLKSGTASVVCLLNNFYPREA |
| light | | KVQWKVDNALQSGNSQESVTEQDSKDSTYSLSSTL |
| chain | | TLSKADYEKHKVYACEVTHQGLSSPVTKSFNRGEC |
| constant | | |
| domain | | |

In any of the above mentioned embodiments, the anti-TIGIT antibody or antigen binding fragment thereof is an antibody comprising any of the variable heavy chains described above and any human heavy chain constant domain. In one embodiment, the antibody or antigen binding fragment thereof of the invention is of the IgG isotype, and comprises a human IgG1, IgG2, IgG3 or IgG4 human heavy chain constant domain. In one embodiment, the antibody or antigen binding fragment thereof of the invention comprises a human heavy chain IgG1 constant domain (SEQ ID NO: 291) or a variant thereof, wherein the variant comprises up to 20 modified amino acid substitutions. In one embodiment, the antibody or antigen binding fragment thereof of the invention is an antibody comprising a human heavy chain IgG1 constant domain comprising the amino acid sequence of SEQ ID NO: 291. In one embodiment, the antibody or antigen binding fragment thereof of the invention comprises a human heavy chain IgG1 constant domain wherein the IgG1 constant domain is afucosylated. In one embodiment, the antibody or antigen binding fragment thereof of the invention comprises a human heavy chain IgG4 constant domain or a variant thereof, wherein the variant comprises up to 20 modified amino acid substitutions. In another embodiment, the antibody or antigen binding fragment thereof of the invention comprises a human heavy chain IgG4 constant domain, wherein the amino acid at position 228 (using EU numbering scheme) has been substituted from Ser to Pro. In one embodiment, the antibody or antigen binding fragment thereof of the invention comprises a human heavy chain IgG4 constant domain comprising the amino acid sequence of SEQ ID NO: 292.

In any of the above mentioned embodiments, the anti-TIGIT antibody or antigen binding fragment thereof can comprise any of the variable light chains described above and human light chain constant domain. In one embodiment, the antibody or antigen binding fragment thereof of the invention comprises a human kappa light chain constant domain or a variant thereof, wherein the variant comprises up to 20 modified amino acid substitutions. In another embodiment, the antibody or antigen binding fragment thereof of the invention comprises a human lambda light chain constant domain or a variant thereof, wherein the variant comprises up to 20 modified amino acid substitutions. In one embodiment, the antibody or antigen binding fragment thereof of the invention comprises a human kappa light chain constant domain comprising the amino acid sequence of SEQ ID NO: 293.

### Formulations

The formulations of the invention minimize the formation of antibody aggregates (high molecular weight species) and particulates, high and low molecular weight species, minimize oxidation of methionine residues, and insure that the antibody retains biological activity over time.

In one aspect, the present invention includes various formulations of an anti-TIGIT antibody, or antigen binding fragment thereof. For example, the present invention includes formulations comprising (i) an anti-TIGIT antibody or antigen binding fragment thereof, (ii) a buffer (e.g., L-histidine or acetate), (iii) a non-reducing sugar (e.g., sucrose); (iv) a non-ionic surfactant (e.g., polysorbate 80); and (v) an antioxidant (e.g., L-methionine). In one embodiment, the formulation further comprises an anti-PD1 antibody. In one embodiment, the formulation further comprises a chelator. In one embodiment, the chelator is present in an amount of about 1 μM to about 50 μM. In one embodiment, the chelator is diethylenetriaminepentaacetic acid (DTPA). In another embodiment, the chelator is EDTA.

In another aspect, the present invention also includes various co-formulations of an anti-TIGIT antibody, or antigen binding fragment thereof and an anti-human PD-1 antibody, or antigen binding fragment thereof. In one embodiment the formulation comprises (i) an anti-TIGIT antibody, or antigen binding fragment thereof, (ii) an anti-human PD-1 antibody or antigen binding fragment thereof, (iii) a buffer (e.g., L-histidine or acetate), (iv) a non-reducing sugar (e.g., sucrose), (v) a non-ionic surfactant (e.g., polysorbate 80), and (vi) an antioxidant (e.g., L-methionine). In one embodiment, the formulation further comprises a chelator In one embodiment, the chelator is present in an amount of about 1 μM to about 50 μM. In one embodiment, the chelator is diethylenetriaminepentaacetic acid (DTPA). In another embodiment, the chelator is EDTA.

Pharmaceutical formulations of the present invention may include buffers.

Buffers that are useful in the pharmaceutical formulations and methods of the invention include succinate (sodium or potassium), L-histidine, phosphate (sodium or potassium), Tris (tris (hydroxymethyl) aminomethane), diethanolamine, citrate (sodium), acetate (sodium) and the like. In an embodiment of the invention, buffer is present in the formulation at a concentration of about 1-20 mM (1, 2, 3, 4, 5, 6, 7, 8, 9, 10, 11, 12, 13, 14, 15, 16, 17, 18, 19 and 20 mM). In specific embodiments of the invention, the buffer is histidine, e.g., L-histidine buffer.

The buffer of this invention has a pH in the range from about 4.5 to about 6.5; about 5.0-6.2; about 5.5-6.0; and preferably has a pH of about 5.8. In arriving at the exemplary formulation, histidine, and acetate buffers in the pH range of 5.0-6.0 were explored for suitability. When a range of pH values is recited, such as “a pH between pH 5.5 and 6.0,” the range is intended to be inclusive of the recited values. For example, a range from about 5.0 to about 6.0 includes 5.0, 5.1, 5.2, 5.3, 5.4, 5.5, 5.6, 5.7, 5.8, 5.9, and 6.0. Unless otherwise indicated, the pH refers to the pH after reconstitution of the lyophilized formulations of the present invention. pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

In an embodiment of the invention, the anti-TIGIT formulation and the co-formulation of anti-TIGIT and anti-human PD-1 comprises a non-reducing sugar. As used herein, “non-reducing sugar” is a sugar not capable of acting as a reducing agent because it does not contain or cannot be converted to contain a free aldehyde group or a free ketone group. Examples of non-reducing sugars include but are not limited to dissacharrides such as sucrose and trehalose. In an embodiment of the invention, the non-reducing sugar is present in an amount of from about 1-10% (w/v) (1, 2, 3, 4, 5, 6, 7, 8, 9 or 10%). In another embodiment, the non-reducing sugar is present in an amount from about 6% to about 8% (w/v) (6, 7, or 8%). In a further embodiment, the non-reducing sugar is present in an amount of about 6% (w/v). In a further embodiment, the non-reducing sugar is present in an amount of about 7% (w/v). In a further embodiment, the non-reducing sugar is present in an amount of about 8% (w/v). In one embodiment, the non-reducing sugar sucrose, trehalose, or raffinose. In another embodiment, the non-reducing sugar is sucrose. In a further embodiment, the sucrose is present at 6-8% w/v. In one embodiment, the sucrose is present at about 6% (w/v). In one embodiment, the sucrose is present at about 7% (w/v). In one embodiment, the sucrose is present at about 8% (w/v).

The formulations of the invention also comprise a surfactant. As used herein, a surfactant is a surface active agent that is amphipathic in nature. Surfactants may be added to the formulations herein to provide stability, reduce and/or prevent aggregation or to prevent and/or inhibit protein damage during processing conditions such as purification, filtration, freeze-drying, transportation, storage, and delivery. In the present invention, a surfactant may be useful for providing additional stability to the active ingredient(s).

Non-ionic surfactants that may be useful in the formulations, including the co-formulation, of the invention includes, but are not limited to, polyoxyethylene sorbitan fatty acid esters (Polysorbates, sold under the trade name Tween® (Uniquema Americas LLC, Wilmington, Del.)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate); polyoxyethylene alkyl ethers such as Brij@ 58 (Uniquema Americas LLC, Wilmington, Del.) and Brij@ 35; poloxamers (e.g., poloxamer 188); Triton® X-100 (Union Carbide Corp., Houston, Tex.) and Triton@ X-114; NP40; Span 20, Span 40, Span 60, Span 65, Span 80 and Span 85; copolymers of ethylene and propylene glycol (e.g., the Pluronic® series of nonionic surfactants such as Pluronic® F68, Pluronic® 10R5, Pluronic® F108, Pluronic® F127, Pluronic® F38, Pluronic® L44, Pluronic® L62 (BASF Corp., Ludwigshafen, Germany); and sodium dodecyl sulfate (SDS). In one embodiment, the non-ionic surfactant is polysorbate 80 or polysorbate 20. In one embodiment, the non-ionic surfactant is polysorbate 20. In another embodiment, the non-ionic surfactant is polysorbate 80.

The amount of non-ionic surfactant to be included in the formulations of the invention is an amount sufficient to perform the desired function, i.e. a minimal amount necessary to stabilize the active pharmaceutical ingredient (i.e. the anti-TIGIT antibody or antigen binding fragment thereof, or both the anti-TIGIT antibody or antigen binding fragment thereof and the anti-human PD-1 antibody or antigen binding fragment thereof) in the formulation. All percentages for the non-ionic surfactant are listed as w/v %. Typically, the surfactant is present in a concentration of from about 0.008% to about 0.1% w/v. In some embodiments of this aspect of the invention, the surfactant is present in the formulation in an amount from about 0.01% to about 0.1%; from about 0.01% to about 0.09%; from about 0.01% to about 0.08%; from about 0.01% to about 0.07%; from about 0.01% to about 0.06%; from about 0.01% to about 0.05%; from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03%. In specific embodiments, the surfactant is present in an amount of about 0.02%. In alternative embodiments, the surfactant is present in an amount of about 0.01%, about 0.015%, about 0.025%, about 0.03%, about 0.035%, or about 0.04%.

In exemplary embodiments of the invention, the surfactant is a nonionic surfactant selected from the group consisting of: Polysorbate 20 and Polysorbate 80. In preferred embodiments, the surfactant is Polysorbate 80.

In specific embodiments, the formulations, including the co-formulations, of the invention comprise about 0.01% to about 0.04% w/v polysorbate 80. In further embodiments, the formulations described herein comprise polysorbate 80 in an amount of about 0.008% w/v, about 0.01% w/v. In one embodiment, the amount of polysorbate 80 is about 0.015 w/v %. In another embodiment, the amount of polysorbate 80 is about 0.02% w/v. In a further embodiment, the amount of polysorbate 80 is about 0.025% w/v. In another embodiment, the amount of polysorbate 80 is about 0.03% w/v. In a further embodiment, the amount of polysorbate 80 is about 0.035% w/v. In another embodiment, the amount of polysorbate 80 is about 0.04% w/v. In a further embodiment, the amount of polysorbate 80 is about 0.045% w/v. In particular embodiments, the formulations of the invention comprise about 0.02% w/v polysorbate 80.

The formulations, including the co-formulations, of the present invention also comprise methionine, or pharmaceutically acceptable salt thereof as an anti-oxidant. In one embodiment, the methionine is L-methionine. In another embodiment, the methionine is a pharmaceutically acceptable salt of L-methionine, such as, for example, methionine HCl. In an embodiment of the invention, methionine is present in the formulation at a concentration of about 1-20 mM (1, 2, 3, 4, 5, 6, 7, 8, 9, 10, 11, 12, 13, 14, 15, 16, 17, 18, 19 and 20 mM). In another embodiment, the methionine is present from about 5 mM to about 10 mM (5, 6, 7, 8, 9 and 10 mM). In another embodiment, the methionine is present at about 10 mM.

The formulations, including the co-formulations, of the present invention may also further comprise a chelating agent. In an embodiment of the invention, chelating agent is present in the formulation at a concentration of about 5-30 μM (e.g., 5, 10, 15, 20, 25, or 30 μM). In one embodiment, the chelating agent is DTPA. In another embodiment, the chelating agent is EDTA.

### Lyophilized Pharmaceutical Compositions

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP, in an exemplary embodiment humanized anti-PD-1 antibody pembrolizumab, or antigen binding fragment thereof), i.e. in anticipation of reconstitution in a low volume of water. Subsequent dilution with water or isotonic buffer can then readily be used to dilute the DP to a lower concentration. Typically, excipients are included in a lyophilized formulation of the present invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous, administration. If isotonicity is desired at lower DP concentration, the lyophilized powder may be reconstituted in the standard low volume of water and then further diluted with isotonic diluent, such as 0.9% sodium chloride.

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the present invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. In one embodiment, the protein concentration after reconstitution is about 10-300 mg/ml. In one embodiment, the protein concentration after reconstitution is about 20-250 mg/ml. In one embodiment, the protein concentration after reconstitution is about 150-250 mg/ml. In one embodiment, the protein concentration after reconstitution is about 180-220 mg/ml. In one embodiment, the protein concentration after reconstitution is about 50-150 mg/ml. In one embodiment, the protein concentration after reconstitution is about 100 mg/ml. In one embodiment, the protein concentration after reconstitution is about 75 mg/ml. In one embodiment, the protein concentration after reconstitution is about 50 mg/ml. In one embodiment, the protein concentration after reconstitution is about 25 mg/ml. High protein concentrations are particularly useful where subcutaneous delivery of the reconstituted formulation is intended. However, for other routes of administration, such as intravenous administration, lower concentrations of the protein may be desired (e.g. from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

### Liquid Pharmaceutical Compositions

A liquid antibody formulation can be made by taking the drug substance (e.g., anti-humanized PD-1) which is in liquid form (e.g., pembrolizumab in an aqueous pharmaceutical formulation) and buffer exchanging it into the desired buffer as the last step of the purification process. There is no lyophilization step in this embodiment. The drug substance in the final buffer is concentrated to a desired concentration. Excipients such as sucrose and polysorbate 80 are added to the drug substance and it is diluted using the appropriate buffer to final protein concentration. The final formulated drug substance is filtered using 0.22 μm filters and filled into a final container (e.g. glass vials).

### III. METHODS OF USE

The invention also relates to a method of treating cancer in a subject, the method comprising administering an effective amount of any of the formulations of the invention; i.e., any formulation described herein, to the subject. In some specific embodiments of this method, the formulation is administered to the subject via intravenous administration. In other embodiments, the formulation is administered to the subject by subcutaneous administration. In one embodiment, the invention comprises a method of treating cancer in a human patient comprising administering any formulation of the invention to the patient.

In any of the methods of the invention, the cancer can be selected from the group consisting of melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g. hormone refractory prostate adenocarcinoma), pancreatic cancer, colon cancer, esophageal cancer, liver cancer, thyroid cancer, glioblastoma, glioma, and other neoplastic malignancies.

In some embodiments the lung cancer in non-small cell lung cancer.

In alternate embodiments, the lung cancer is small-cell lung cancer.

In some embodiments, the lymphoma is Hodgkin lymphoma.

In other embodiments, the lymphoma is non-Hodgkin lymphoma. In particular embodiments, the lymphoma is mediastinal large B-cell lymphoma.

In some embodiments, the breast cancer is triple negative breast cancer.

In further embodiments, the breast cancer is ER+/HER2− breast cancer.

In some embodiments, the bladder cancer is urothelial cancer.

In some embodiments, the head and neck cancer is nasopharyngeal cancer. In some embodiments, the cancer is thyroid cancer. In other embodiments, the cancer is salivary cancer. In other embodiments, the cancer is squamous cell carcinoma of the head and neck.

In one embodiment, the invention comprises a method of treating metastatic non-small cell lung cancer (NSCLC) in a human patient comprising administering a formulation of the invention to the patient. In specific embodiments, the patient has a tumor with high PD-L1 expression [(Tumor Proportion Score (TPS) ≥50%)] and was not previously treated with platinum-containing chemotherapy. In other embodiments, the patient has a tumor with PD-L1 expression (TPS ≥1%) and was previously treated with platinum-containing chemotherapy. In still other embodiments, the patient has a tumor with PD-L1 expression (TPS ≥1%) and was not previously treated with platinum-containing chemotherapy. In specific embodiments, the patient had disease progression on or after receiving platinum-containing chemotherapy. In certain embodiments, the PD-L1 TPS is determined by an FDA-approved test. In certain embodiments, the patient's tumor has no EGFR or ALK genomic aberrations. In certain embodiments, the patient's tumor has an EGFR or ALK genomic aberration and had disease progression on or after receiving treatment for the EGFR or ALK aberration(s) prior to receiving the anti-PD-1 antibody, or antigen binding fragment thereof.

In some embodiments, the cancer is metastatic colorectal cancer with high levels of microsatellite instability (MSI-H).

In some embodiments, the cancer is metastatic colorectal cancer with high levels of microsatellite instability (MSI-H).

In some embodiments, the cancer is a solid tumor with a high level of microsatellite instability (MSI-H).

In some embodiments, the cancer is a solid tumor with a high mutational burden.

In some embodiments, the cancer is selected from the group consisting of: melanoma, non-small cell lung cancer, relapsed or refractory classical Hodgkin lymphoma, head and neck squamous cell carcinoma, urothelial cancer, esophageal cancer, gastric cancer, and hepatocellular cancer.

In other embodiments of the above treatment methods, the cancer is a Heme malignancy.

In certain embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin lymphoma (NHL), or small lymphocytic lymphoma (SLL).

Malignancies that demonstrate improved disease-free and overall survival in relation to the presence of tumor-infiltrating lymphocytes in biopsy or surgical material, e.g. melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

Additional cancers that can benefit from treatment with the formulations described herein include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile and oral cancers.

The formulations can also be used to prevent or treat infections and infectious disease. Thus, the invention provides a method for treating chronic infection in a mammalian subject comprising administering an effective amount of a formulation of the invention to the subject. In some specific embodiments of this method, the formulation is administered to the subject via intravenous administration. In other embodiments, the formulation is administered to the subject by subcutaneous administration.

These agents can be used alone, or in combination with vaccines, to stimulate the immune response to pathogens, toxins, and self-antigens. The antibodies or antigen-binding fragment thereof can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-CTL4 antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist.

Suitable routes of administration may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g. subcutaneous administration) require a lyophilized formulation to enable reconstitution at high concentration.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g. biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J Med. 348:601-608; Milgrom et al. (1999) New Engl. J Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J Med. 348:24-32; Lipsky et al. (2000) New Engl. J Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, Pa. (1984); Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

A pharmaceutical antibody formulation can be administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002)New Engl. J. Med. 346:1692-1698; Liu et al. (1999) J Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

Embodiments of the invention also include one or more of the biological formulations described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of PD-1-related disease or an anti-TIGIT related disease; (g) halting or delaying the progression cancer; (h) stabilization of PD-1-related disease or an anti-TIGIT disease; (i) inhibiting the growth or survival of tumor cells; (j) eliminating or reducing the size of one or more cancerous lesions or tumors; (k) reduction of the progression, onset or severity of PD-1-related disease or an anti-TIGIT disease; (1) reducing the severity or duration of the clinical symptoms of PD-1-related or anti-TIGIT related disease such as cancer (m) prolonging the survival of a patient relative to the expected survival in a similar untreated patient n) inducing complete or partial remission of a cancerous condition or other PD-1 related or anti-TIGIT related disease, o) treatment of cancer, or p) treatment of chronic infections.

### GENERAL METHODS

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391).

Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J Immunol. 165:6205; He, et al. (1998) J Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J Immunol. 160:3891-3898; Hsing and Bishop (1999) J Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

### Example 1

### Anti-TIGIT Formulation Buffer Screening

High throughput formulation development study was performed for three anti-TIGIT antibodies, each anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113, to evaluate (1) biophysical/biochemical liabilities; (2) pre-formulation (pH, salt and buffer) conditions and (3) compatibility with platform formulation across. The samples were analyzed by UV/Vis spectrophotometry for turbidity (A350) as surrogate of larger aggregates, size exclusion chromatography (UP-SEC) to detect the formation of high molecular weight species, capillary isoelectric focusing (cIEF) to measure the effect of stress on distribution of charges at the surface of the molecule, reducing sodium dodecyl sulfate capillary electrophoresis (CE-SDS) to detect proteolytic cleavage of the heavy or light chains, sub-visible particle analysis to detect sub-visible aggregates.

The high throughput formulation screen comprised of 1 mg/mL anti-TIGIT antibody formulated in a selection of three buffer species: Acetate buffer with pH values ranging from 5.0 to 6.2, Citrate buffer with pH values ranging from 5.6 to 6.8 and L-histidine buffer with pH values ranging from 5.0 to 6.8. Thus, pH values ranging from 5.0 to 6.8 and ionic strength from 0-150 mM NaCl was examined. The samples were stressed at 50° C. for 10 days and analyzed for thermal stability using Differential Scanning Fluorescence (DSF), colloidal stability using Size-exclusion chromatography (UP-SEC), aggregation propensity using Guava (a flow-cytometry based sub-visible characterization assay), turbidity (A350 measurements), charge variants profile (cIEF), and fragmentation profile using Caliper.

Based on the results obtained from the study, the formulation, which imparted the maximum stability to the protein was 10 mM L-Histidine in the pH range of 5.6 to 6.2. 10 mM L-Histidine in the pH range of 5.6 to 6.2 showed minimal aggregation monitored by UP-SEC with ASEC Main ranging between −1.63 to −1.85% (compared to SEC Main ranging from −2.0 to −5.0% in other buffer and pH conditions) (data not shown). cIEF profile showed decrease of relative peak area of the main and basic species and increase of the relative peak area of the acidic variants was noted for samples after 10 days at 50° C. (cIEF Main ranging between −9.0 to −11.3%) (data not shown). A decrease of basic variants and increase of acidic variants upon exposure to elevated temperature is a common occurrence for mAbs. Addition of salt reduced the stability of the protein across studied compositions.

### Example 2

### Anti-TIGIT Formulation pH Ranging Study

In this study, an anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113, was tested at 50 mg/mL concentration in 10 mM L-histidine buffer. 7% (w/v) Sucrose was added to the formulation to increase the bulk stability (as stabilizer and non-ionic tonicity modifier) of the molecule. The anti-TIGIT antibody was formulated in 10 mM L-Histidine buffer, 7% Sucrose at pH 5.5, pH 6.0 and pH 6.5. Stability of such formulations was evaluated as follows:
(1) The stability of the molecule was monitored under accelerated thermal and storage stability conditions (5° C., 25° C. and 40° C. for up to 6 months), protected from light.
(2) Stability studies for freeze-thaw stress and agitation stress were also conducted.
(3) Agitation study was conducted in formulations containing varying concentrations of polysorbate 80 (PS-80) to assess the concentration of PS-80 in the formulation.
(4) Light stress study was conducted to evaluate the requirement of L-methionine in the formulations.

### Materials and Methods

### Thermal Stability Study (3 Months)

50 mg/mL anti-TIGIT antibody was formulated in 10 mM L-Histidine buffer, 7% Sucrose, 0.2 mg/mL polysorbate 80 at pH 5.5, pH 6.0 or pH 6.5. The resulting formulations were sterile filtered and filled in 2R vials, stoppered with chlorobutyl stoppers and capped with aluminum caps with seals. The stability study was staged at 5° C. (ambient humidity), 25° C. (60% relative humidity) and 40° C./(75% relative humidity). The samples were analyzed using UP-SEC, HP-IEX, cIEF (select samples), MFI, CE-SDS (non-reduced “NR” and reduced “R”), reduced peptide mapping (MFI and reduced peptide mapping was performed at select time points).

A 1 month thermal stability study was set up for 25 mg/ml anti-TIGIT antibody formulated in 10 mM L-histidine buffer, 7% sucrose, 0.2 mg/ml PS-80, pH 6.0. The resulting formulation was sterile filtered and filled in 2R vials, stoppered with chlorobutyl stoppers and capped with aluminum seals. The stability study was staged at 5° C. (ambient humidity), 25° C. (60% relative humidity) and 40° C./(75% relative humidity) for one month. The samples were analyzed using UP-SEC, cIEF, and CE-SDS (NR and R).

### Agitation Stability Study

50 mg/mL anti-TIGIT antibody was formulated in 10 mM L-Histidine buffer, 7% Sucrose, pH 6.0 with various concentration of polysorbate 80 (0, 0.1, and 0.2 mg/mL). The resulting formulations were sterile filtered and filled in 2R vials (1.2 mL fill volume), stoppered with chlorobutyl stoppers and capped with aluminum caps with seals. The samples were agitated in a horizontal position at 300 RPM for up to 7 days at 18-22° C. The samples were analyzed using UP-SEC, MFI, CE-SDS (NR and R).

### Freeze-Thaw Stability

50 mg/mL anti-TIGIT antibody was formulated in 10 mM L-Histidine buffer, 7% Sucrose, 0.2 mg/mL polysorbate 80, pH 6.0. The resulting formulation was sterile filtered and filled in 2R vials, stoppered with chlorobutyl stoppers and capped with aluminum caps with seals. The samples were subjected to 5 freeze-thaw cycle at −80° C. to 18-22° C. (at least 24 hours at frozen conditions and at room temperature until thawed completely). The samples were analyzed using UP-SEC, MFI, CE-SDS (NR and R).

### Light Stress Stability Study

Early development studies indicated the presence of an exposed tryptophan residue as well as some methionines that were liable for oxidation under light stress. Studies were set up under ICH light stress conditions visible light (CWF, 0.1×ICH, 0.2×ICH, 0.5×ICH, 1×ICH) in formulations with and without L-methionine (Formulation 1: 10 mM L-Histidine buffer, 7% Sucrose, 0.2 mg/mL polysorbate 80, pH 6.0 and Formulation 2: 10 mM L-Histidine buffer, 10 mM L-methionine, 7% Sucrose, 0.2 mg/mL polysorbate 80, pH 6.0). The samples were analyzed using UP-SEC, cIEF, CE-SDS (NR and R) and reduced peptide mapping.

### Results

### Thermal Stability Results

50 mg/ml formulation: For all the tested stability indicating assays, no significant changes were observed at 5° C. at all the pH values after 3 months (data not shown). At 25° C. and 40° C., pH 5.5 and pH 6.0 showed similar stability and these conditions were more stable than pH 6.5 (data not shown). The degradation rates at pH 6.5 for UP-SEC, CE-SDS (NR) and cIEF assays at 25° C. and 40° C. were relatively higher than that seen at pH 5.5 and pH 6.0 and also when compared to the benchmark molecule. Hence, a pH range of 5.5 to 6.0 (pI of 8.7) was considered suitable. No oxidation, deamidation or isomerization was observed for the anti-TIGIT antibody after 3 months at all temperatures and pH values.

25 mg/ml formulation: Degradation was observed at 25° C. and 40° C. for all the tested assays. Degradation rates were found to be similar to 50 mg/ml conditions (see above).

### Agitation Stability Study

Formulations which did not contain polysorbate 80 showed visible particles at the end of 7 days. Subvisible particle analysis showed that particles 10 μm or greater were significantly reduced in the formulation containing 0.2 mg/mL polysorbate 80 concentration. Significant differences between samples were not seen using the other assays.

### Freeze-Thaw Stability

No changes were seen in the stability of the molecule after 5 cycles of freeze-thaw in all of the tested assays.

### Light Stress Stability Study

Degradation was observed when samples of both formulations were exposed to light stress at or above 0.5×ICH when tested by UP-SEC, cIEF, CE-SDS (NR and R). Conditions setup below 0.5×ICH did not show significant degradation for both formulations. Reduced peptide mapping data under light stress conditions 0.5× and above showed oxidation of tryptophan and methionine residues. 10 mM L-methionine in the formulation reduced the levels of oxidation of the methionine residues but did not impact the oxidation levels of tryptophan.

### Conclusion

Based on the foregoing, 10 mM L-Histidine buffer, 10 mM L-methionine, 7% Sucrose, 0.2 mg/mL polysorbate 80 pH 5.5-6.0 is considered adequate to impart stability to support shelf life under refrigerated conditions.

### Example 3

### Additional pH Studies

An anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113 and on an IgG1 backbone was formulated in six 10 mM histidine buffers with different pH (ranging from 5.0 to 6.5). The thermal stability in different formulations was studied at 2-8° C., 25° C. and 40° C. over 8 weeks.

| | Anti- | | | | | |
|---|---|---|---|---|---|---|
| Formu- | TIGIT | | | | Sucrose | Polysorbate |
| lation | antibody | | Buffer | pH | (% w/v) | 80 (% w/v) |
| 1 | 50 | mg/Ml | 10 mM | pH = 5.0 | 7% | 0.02% |
| | | | L-Histidine | | | |
| 2 | 50 | mg/mL | 10 mM | pH = 5.3 | 7% | 0.02% |
| | | | L-Histidine | | | |
| 3 | 50 | mg/mL | 10 mM | pH = 5.6 | 7% | 0.02% |
| | | | L-Histidine | | | |
| 4 | 50 | mg/mL | 10 mM | pH = 5.9 | 7% | 0.02% |
| | | | L-Histidine | | | |
| 5 | 50 | mg/mL | 10 mM | pH = 6.2 | 7% | 0.02% |
| | | | L-Histidine | | | |
| 6 | 50 | mg/mL | 10 mM | pH = 6.5 | 7% | 0.02% |
| | | | L-Histidine | | | |

Histidine buffers of different pH (5.0˜6.5) were prepared by titrating 10 mM L-histidine buffer into 10 mM L-histidine-HCl buffer. The anti-TIGIT antibody was buffer exchanged into six different histidine buffers with different pH through four to five rounds of ultrafiltration using the centrifuge device under the condition of 4° C. and 4500 rpm-5000 rpm (105-260 min in each round). After buffer exchange, the specific amount of sucrose and polysorbate 80 stock solution (1%, w/w) was added to solutions of different pH to reach the target amount and appropriate amount of corresponding histidine buffer was added as well to adjust the antibody concentration to around 50 mg/ml.

The formulations were then aseptically filtered with 0.22-μm membrane filter. 3 mL of each sample was aseptically filled into 6-mL glass vials for the T0, 4 week (4 W) and 8 week (8 W) thermal stability study. 1 mL of each sample was aseptically filled into 6-mL glass vials for the two week (2 W) thermal stability study. The filled vials were stoppered and crimp-over-sealed immediately after the filling. All the above steps were performed in bio-safety hood.

Those vials were put into covered boxes and stored in different temperature conditions for thermal stability study.

### Results and Discussion

The appearance of all samples remained the same within four weeks at all conditions. However, after 8 weeks, samples at 2-8° C. and 25° C. showed slightly yellowish and samples at 40° C. showed deeper yellowish. All samples were slightly opalescent and free of visible particles during the study period. A considerable change of protein concentration of all samples was not seen during study.

The colloidal stability of the samples were assessed by size exclusion chromatography (SEC) for purity in which the percentage of monomer, the percentages of high molecular weight species (HMW), and late eluting peaks (LMW species). The analysis was performed using an Agilent 1260 Infinity system with the TSKGel G3000SWXL size exclusion chromatography column (300×7.8 mm, 5 μm). The mobile phase was 50 mM PB, 300 mM NaCl, pH 7.0±0.2 and the flow rate was set at 1.0 mL/min. Samples were diluted to 10 mg/mL for injection and detected at 280 nm with a UV detector.

The UPSEC data is set forth in the table below:

| | | | 2-8° C. | | | 25° C. | | | 40° C. | | |
|---|---|---|---|---|---|---|---|---|---|---|---|
| | pH | T0 | 2 W | 4 W | 8 W | 2 W | 4 W | 8 W | 2 W | 4 W | 8 W |
| Main | 5 | 97.7 | 97.5 | 97.4 | 97.5 | 97.7 | 97.4 | 97 | 97.2 | 96 | 94.5 |
| Peak | 5.3 | 97.7 | 97.5 | 97.7 | 97.4 | 97.6 | 97.2 | 96.8 | 96.5 | 96.2 | 94.6 |
| % | 5.6 | 97.6 | 97.4 | 97.6 | 97.3 | 97.1 | 97.1 | 96.8 | 97 | 96.1 | 94.8 |
| | 5.9 | 97.5 | 97.3 | 97.5 | 97.3 | 97.3 | 97 | 96.5 | 96.2 | 96 | 94.7 |
| | 6.2 | 97.3 | 97.2 | 97.5 | 97.1 | 97.1 | 96.8 | 96.5 | 96.4 | 95.7 | 94.3 |
| | 6.5 | 97.3 | 97.1 | 97.4 | 97.1 | 96.9 | 96.6 | 96.2 | 95.8 | 95.5 | 94.6 |
| % | 5 | 2.3 | 2.5 | 2.6 | 2.5 | 2.3 | 2.4 | 2.8 | 2.4 | 2.8 | 3.4 |
| HMW | 5.3 | 2.3 | 2.5 | 2.3 | 2.6 | 2.4 | 2.5 | 2.9 | 2.8 | 2.9 | 3.7 |
| | 5.6 | 2.4 | 2.6 | 2.4 | 2.7 | 2.7 | 2.7 | 3 | 2.8 | 3.2 | 3.9 |
| | 5.9 | 2.5 | 2.7 | 2.5 | 2.7 | 2.7 | 2.8 | 3.2 | 3.3 | 3.3 | 4.1 |
| | 6.2 | 2.7 | 2.8 | 2.5 | 2.9 | 2.9 | 3 | 3.3 | 3.3 | 3.6 | 4.6 |
| | 6.5 | 2.7 | 2.9 | 2.6 | 2.9 | 3.1 | 3.2 | 3.5 | 3.7 | 3.9 | 4.3 |
| % | 5 | N.D. | N.D. | N.D. | N.D. | N.D. | 0.2 | 0.3 | 0.4 | 1.3 | 2.1 |
| LMW | 5.3 | N.D. | N.D. | N.D. | N.D. | N.D. | 0.2 | 0.2 | 0.6 | 0.8 | 1.7 |
| | 5.6 | N.D. | N.D. | N.D. | N.D. | 0.2 | 0.2 | 0.2 | 0.2 | 0.7 | 1.3 |
| | 5.9 | N.D. | N.D. | N.D. | N.D. | N.D. | 0.2 | 0.3 | 0.5 | 0.7 | 1.2 |
| | 6.2 | N.D. | N.D. | N.D. | N.D. | N.D. | 0.2 | 0.2 | 0.3 | 0.7 | 1.1 |
| | 6.5 | N.D. | N.D. | N.D. | N.D. | N.D. | 0.2 | 0.2 | 0.5 | 0.7 | 1.1 |

As shown in the table above, the SEC main peak % was stable at 2-8° C. in all samples, however, at 25° C. and 40° C., significant main peak % decrease was observed. The rate for main peak % decrease was faster in samples at 40° C. than that at 25° C. At 40° C. for eight weeks, the HMW % was larger in pH6.2 and 6.5 samples, while the LMW % was larger in pH5.0 and 5.3 samples.

To evaluate the chemical stability of the formulations, capillary isoelectric focusing (cIEF) was performed to evaluate the chemical stability and to monitor the change in the charge variant profile over time. In brief, 20 μL (2.0 mg/mL) of reference standard or the sample was mixed with 0.5 μL of pI 5.85 marker, 0.5 μL of pI 9.77 marker, 1 μL of Pharmalyte 3-10, 0.5 μL of Pharmalyte 5-8, 0.5 μL of Pharmalyte 8-10.5, 35 μL of 1% methyl cellulose, 37.5 μL of 8 M urea. Purified water was added to make up a final volume of 100. The mixture was then analyzed with iCE-3 capillary isoelectric focusing analyzer equipped with a fluorocarbon-coated whole-column detection capillary. The focusing was carried out by two steps: (1) 1.5 kV for 1 min, and (2) 3 kV for 8 min. During the experiment, the auto-sampler tray was maintained at 5° C.

The cIEF data to evaluate the levels of Acidic Variants, % Main Peak and % Basic Variants is in the Table below.

| | | | 2-8° C. | | | 25° C. | | | 40° C. | | |
|---|---|---|---|---|---|---|---|---|---|---|---|
| | pH | T0 | 2 W | 4 W | 8 W | 2 W | 4 W | 8 W | 2 W | 4 W | 8 W |
| Main | 5 | 62.2 | 63 | 60 | 62.6 | 62.8 | 57.6 | 58.4 | 50.2 | 37.5 | 29.1 |
| Peak | 5.3 | 62.2 | 63 | 59.9 | 62.4 | 62.8 | 57.7 | 58.6 | 50.6 | 39.1 | 30.2 |
| % | 5.6 | 62.3 | 62.8 | 59.9 | 62.8 | 62.6 | 58.1 | 58.7 | 51.9 | 41.4 | 31 |
| | 5.9 | 62 | 62.9 | 59.7 | 62.5 | 62.8 | 58.1 | 58.4 | 51.7 | 41.2 | 32.4 |
| | 6.2 | 62.1 | 62.8 | 59.6 | 62.2 | 62.5 | 57.5 | 58.6 | 51.7 | 41.8 | 31.8 |
| | 6.5 | 62.2 | 62.9 | 59.4 | 62.5 | 61.9 | 56.1 | 57.4 | 51.1 | 40.4 | 33.2 |
| Acidic | 5 | 31.7 | 30.3 | 32.7 | 30.2 | 30.5 | 33.5 | 33.9 | 39.7 | 49.2 | 58.2 |
| % | 5.3 | 31.8 | 30.6 | 32.3 | 30.6 | 30.5 | 34 | 34.1 | 40.3 | 49.5 | 59.4 |
| | 5.6 | 31.4 | 30.8 | 32.6 | 30.3 | 30.8 | 34.2 | 34.3 | 39.8 | 48.9 | 59.5 |
| | 5.9 | 31.9 | 30.7 | 33.1 | 30.6 | 30.9 | 34.3 | 34.8 | 40.7 | 48.8 | 59.1 |
| | 6.2 | 32.1 | 30.8 | 33.5 | 31.2 | 31.3 | 34.5 | 34.6 | 41 | 49 | 59.7 |
| | 6.5 | 31.5 | 31 | 33.3 | 31 | 31.9 | 35.5 | 35.8 | 41.5 | 50.3 | 58.5 |
| Basic | 5 | 6 | 6.7 | 7.3 | 7.2 | 6.8 | 8.9 | 7.7 | 10 | 13.3 | 12.6 |
| % | 5.3 | 6 | 6.4 | 7.7 | 7 | 6.7 | 8.3 | 7.3 | 9.1 | 11.3 | 10.3 |
| | 5.6 | 6.2 | 6.5 | 7.5 | 6.8 | 6.5 | 7.6 | 7.2 | 8.2 | 9.6 | 9.5 |
| | 5.9 | 6.1 | 6.4 | 7.3 | 6.9 | 6.3 | 7.5 | 6.7 | 7.6 | 9.9 | 8.4 |
| | 6.2 | 5.9 | 6.4 | 6.9 | 6.6 | 6.3 | 8 | 6.8 | 7.2 | 9.3 | 8.6 |
| | 6.5 | 6.3 | 6.2 | 7.2 | 6.4 | 6.2 | 8.4 | 6.8 | 7.3 | 9.4 | 8.3 |

As seen in the table above, at 2-8° C., the cIEF main peak %, acid peak % and basic peak % were relatively stable and comparable in all samples.

At 2-8° C., the cIEF main basic peak % was also increased; the increase in pH 5.0 and 5.3 buffer samples was greater than those in other samples. At 25° C., the main peak %, acid peak % and basic peak % was stable within the first two weeks but slightly changed after four weeks where the main peak % decreased while the acid peak % increased correspondingly). The change rate in different formulation was comparable. At 40° C., significant decrease in main peak % and noteworthy increase in acid peak % were found in all formulations even after two weeks but the change extent was similar in each formulation. The basic peak % was also increased; the increase in pH 5.0 and 5.3 buffer samples was greater than those in other samples.

To evaluate the purity of the formulations, non-reduced Caliper analysis was carried out. In brief, the purchased sample buffer was mixed with 10% Sodium Dodecyl Sulfate (SDS) solution at a volume ratio of 20 to 1, and 100 mM N-ethylmaleimide solution was added to the mixed solution at a volume ratio of 0.7 to 20 (referred to the sample denaturing solution). The standard or sample was diluted to 1 mg/mL first, and 2 μL of diluted standard or sample was mixed with 7 μL of sample denaturing solution. The mixture was incubated at 70° C. for 10 min. 35 μL of purified water was added to the incubated solution, and 42 μL of the mixed solution was transferred to a 96-well plate for analyzing. The sample plate was analyzed with the LabChip GX II HT, using the HT Antibody Analysis 200 assay.

The non-reduced Caliper analysis data to evaluate the % purity is shown in the Table below:

| | | 2-8° C. | | | 25° C. | | | 40° C. | | |
|---|---|---|---|---|---|---|---|---|---|---|
| pH | T0 | 2 W | 4 W | 8 W | 2 W | 4 W | 8 W | 2 W | 4 W | 8 W |

Caliper Non-Reduced

| | | | | | | | | | | | |
|---|---|---|---|---|---|---|---|---|---|---|---|
| Purity | 5 | 96 | 95.2 | 94.6 | 94.9 | 94.9 | 93.6 | 94.1 | 91.4 | 87.8 | 84.4 |
| % | 5.3 | 96 | 95 | 94.7 | 94.9 | 94.8 | 93.8 | 94.1 | 92.7 | 89.5 | 86.4 |
| | 5.6 | 96.1 | 95 | 94.6 | 94.9 | 94.8 | 93.9 | 94.1 | 93 | 90.7 | 88.5 |
| | 5.9 | 96.3 | 94.9 | 94.5 | 94.9 | 94.8 | 93.9 | 94.2 | 93.4 | 91.1 | 89.3 |
| | 6.2 | 96.2 | 94.9 | 94.5 | 95 | 94.8 | 93.7 | 94.5 | 93.3 | 91.3 | 88.7 |
| | 6.5 | 96 | 95.1 | 94.4 | 94.9 | 94.7 | 93.4 | 94.1 | 93.2 | 90.7 | 89.3 |

As shown in the Table above—at 2˜8° C. for eight weeks, the Caliper_Non-reduced purity in each formulation was relatively stable. At 25° C. for eight weeks, the purity in each formulation was decreased slightly. At 40° C., the purity declined significantly, especially when samples were in pH 5.0 and 5.3 buffer, the decrease was much faster than that of others. The molecular size was stable during the study (data not shown).

To further evaluate the purity of the formulations, reduced Caliper analysis was also carried out. In brief, the purchased sample buffer was mixed with 10% SDS solution at a volume ratio of 20 to 1, and 1M dithiothreitol solution was added to the mixed solution at a volume ratio of 0.7 to 20 (referred to the sample denaturing solution). The standard or sample was diluted to 1 mg/mL first, and 2 μL of diluted standard or sample was mixed with 7 μL of sample denaturing solution. The mixture was incubated at 70° C. for 10 min. 35 μL of purified water was added to the incubated solution, and 42 μL of the mixed solution was transferred to a 96-well plate for analyzing. The sample plate was analyzed with the LabChip GX II HT, using the HT Antibody Analysis 200 assay.

The reduced Caliper analysis data to evaluate the % purity is shown in the Table below:

| | | | 2-8° C. | | | 25° C. | | | 40° C. | | |
|---|---|---|---|---|---|---|---|---|---|---|---|
| | pH | T0 | 2 W | 4 W | 8 W | 2 W | 4 W | 8 W | 2 W | 4 W | 8 W |
| Purity % | 5 | 99.4 | 99.4 | 99.3 | 99.2 | 99.3 | 99.1 | 98.9 | 98 | 97.1 | 91.3 |
| (Caliper_R) | 5.3 | 99.4 | 99.4 | 99.2 | 99.2 | 99.3 | 99 | 99 | 98.5 | 97.7 | 94.9 |
| | 5.6 | 99.4 | 99.4 | 99.3 | 99.1 | 99.3 | 99.2 | 98.8 | 98.7 | 98.2 | 96.8 |
| | 5.9 | 99.4 | 99.4 | 99.2 | 99.1 | 99.2 | 99.2 | 98.8 | 99.1 | 98.6 | 96.8 |
| | 6.2 | 99.3 | 99.3 | 99.3 | 99.2 | 99.3 | 99.2 | 98.9 | 98.9 | 98.7 | 96.9 |
| | 6.5 | 99.3 | 99.4 | 99.2 | 99.1 | 99.3 | 99.2 | 98.8 | 98.9 | 98.5 | 97.5 |

As seen above, at 2˜8° C. and 25° C. for eight weeks, the Caliper_Reduced purity in each formulation was relatively stable. At 40° C., an apparent decline in Caliper_R purity was found in all formulations. The purity of samples in pH 5.0 buffer had the greatest decrease, followed by samples in pH 5.3 buffer. The decrease rate in pH 5.6, 5.9 and 6.2 buffers were comparable but slower. The purity of samples in pH 6.5 buffer had the slowest decrease. The purity decline was likely due to the decrease in the heavy chain (HC) % while the light chain (LC) % was stable in the study. The antibody light chain and heavy chain size were stable in all samples over 8 W.

### Example 4

Anti-TIGIT Formulation without Methionine

An anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113, was tested at 50 mg/mL concentration in 10 mM L-Histidine buffer, 7% Sucrose, 0.2 mg/mL PS-80 with pH ranging from 5.0 to 6.5. The stability of the molecule was monitored under accelerated thermal and storage stability conditions, protected from light. In addition to thermal stability, freeze-thaw stability, agitation stability, light stress stability studies were also conducted. Stability was tested including UP-SEC, cIEF, CE-SDS, MFI and reduced peptide mapping.

### Results

### Thermal Stability Study (8 Weeks)

For all the tested stability indicating assays, the anti-TIGIT antibody was stable at 5° C. for all the tested liabilities. The degradation rates as observed using UP-SEC, Caliper CE-SDS, cIEF, MFI were higher at 40° C. than at 25° C. At 25° C. and 40° C., the following results were notable:

UP-SEC: A decline in % monomer was observed for all pH values from 5.0 to 6.5. At the lower pH values (5.0 and 5.3), the main peak decline was primarily due to increase in % low molecular weight (LMW) species whereas the % monomer decline at pH 6.5 was mainly due to the increase in % high molecular weight (HMW) species. The % monomer decline was highest at pH 6.5 after 8 weeks of accelerated stability. (data not shown)

cIEF: cIEF main peak decline was observed for all pH values from 5.0 to 6.5 at 25° C. and 40° C. At the higher pH values (6.3-6.5), the main peak decline was mainly due to the increase in acidic variants whereas at pH 5.0 and 5.3, the main peak decline was due to the increase in both acidic as well as basic variants. (data not shown)

CE-SDS (Caliper): The non-reduced CE-SDS main peak decline was mainly observed at 40° C. due to the presence of fragmented species. Formulations at lower pH values (5.0 and 5.3) showed a relatively higher rate of fragmentation than the rest of the pH values.

Reduced CE-SDS (Caliper) analysis revealed that at 5° C. and 25° C. for eight weeks, the purity in each formulation was relatively stable. At 40° C., an apparent decline in purity was found in all formulations. The purity of samples in pH 5.0 buffer had the greatest decrease, followed by samples in pH 5.3 buffer.

MFI: Sub-visible particle increases were observed for all formulations at 40° C. pH 6.3 and 6.5 had the highest increases in sub-visible particles relative to the rest of the buffers.

Reduced peptide mapping: Among the identified liabilities, only M254 showed a relative increase in oxidation after 8 weeks at 40° C. relative to the initial samples.

Conclusion: 10 mM L-Histidine buffer, 7% Sucrose, 0.2 mg/mL PS-80, pH 5.6-6.3 adequately supported storage stability of the anti-TIGIT antibody over 8 weeks.

### Agitation Stability Study

No changes in soluble aggregates, charged variants, fragmentation or subvisible particles were observed when the 50 mg/mL anti-TIGIT formulation (in 10 mM L-Histidine buffer, 7% Sucrose, pH 5.8 and either 0, 0.1, 0.2 and 0.3 mg/mL PS-80) was mildly agitated for up to 7 days 100 RPM at 18-22° C.

### Freeze-Thaw Stability

No changes in soluble aggregates, charged variants, fragmentation or subvisible particles were observed when 50 mg/mL of the anti-TIGIT antibody (in 10 mM L-Histidine buffer, 7% Sucrose, 0.2 mg/mL PS-80, pH 5.8) upon 5 cycles of freeze/thaw (frozen at −80° C. for 2 hours and thawed at room temperature for 1 hour).

### Light Stress Stability Study

The 50 mg/ml formulation was subjected to 48 hours under visible light stress (5000 lx). Under these conditions, there was minimal change in soluble aggregates, charged variants, subvisible particles, pH, concentration, fragmentation and oxidation under ˜0.2×ICH conditions (12H light exposure). Under ˜1× (48H light exposure) conditions, there was an increase in soluble aggregates, acidic variants, fragmentation, subvisible particles and methionine oxidation.

### Conclusion

Based on these studies, 10 mM L-Histidine buffer, 7% Sucrose, 0.2 mg/mL PS-80 pH 5.3-6.3 was able to support the stability of the anti-TIGIT antibody. Methionine oxidation was observed upon exposure to severe light stress. As noted in Example 2, the addition of 10 mM L-methionine reduces oxidation of the methionine residues.

### Example 5

### Polysorbate 80 Screening

An anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113, was formulated into four (4) formulations of pH 5.8, 10 mM L-histidine buffer, and with different PS-80 concentrations (as shown below). The protein stability in different formulations was studied in condition of with or without agitation over 7-day period at 20° C.

| Formu- | anti-TIGIT | | | | Sucrose | Polysorbate 80 | |
|---|---|---|---|---|---|---|---|
| lation | antibody | | Buffer | pH | (% w/v) | (mg/ml) | |
| 1 | 50 | mg/mL | 10 mM L-Histidine | pH = 5.8 | 7% | 0 | mg/mL |
| 2 | 50 | mg/mL | 10 mM L-Histidine | pH = 5.8 | 7% | 0.1 | mg/mL |
| 3 | 50 | mg/mL | 10 mM L-Histidine | pH = 5.8 | 7% | 0.2 | mg/mL |
| 4 | 50 | mg/mL | 10 mM L-Histidine | pH = 5.8 | 7% | 0.3 | mg/mL |

The formulations were formulated 10 mM L-histidine buffer at pH 5.8 using a lab-scale TFF buffer exchange system. The formulated proteins with different polysorbate 80 content were then aseptically filtered with 0.22-μm membrane filter. 2 mL of each sample was then aseptically filled into 6-mL glass vials. The filled vials were stoppered and crimp-over-sealed immediately after filling. Samples were divided into agitation group and non-agitation group. In the agitation group, those vials were transferred to covered boxes and then put in the thermostat shaker and agitated at 100 rpm, 20° C. for up to 7 days. In the non-agitation group, those vials were transferred to covered boxes and put in the thermostat shaker but the shaker was kept still at 20° C. for up to 7 days.

The antibody stability in the different formulations with or without agitation was studied after 3 and 7 days.

The UPSEC data to evaluate the levels of High Molecular Weight Species (HMW or aggregates), % monomer and LMW (Low Molecular Weight species) is in the Table below:

| | | | 3 days | | 7 days | |
|---|---|---|---|---|---|---|
| | Formu- | | Non- | | Non- | |
| | lation | T0 | agitation | 100 rpm | agitation | 100 rpm |
| Main | 1 | 97.1 | 97.2 | 97.2 | 97.1 | 97.2 |
| Peak % | 2 | 97 | 97.1 | 97.1 | 97 | 97 |
| | 3 | 97 | 97.1 | 97.1 | 96.9 | 96.9 |
| | 4 | 97.1 | 97.1 | 97.1 | 96.9 | 96.9 |
| High | 1 | 2.9 | 2.8 | 2.8 | 2.9 | 2.8 |
| Molecular | 2 | 3 | 2.9 | 2.9 | 3 | 3 |
| Weight | 3 | 3 | 2.9 | 2.9 | 3.1 | 3.1 |
| (HMW) % | 4 | 2.9 | 2.9 | 2.9 | 3.1 | 3.1 |
| Low | 1 | N.D. | N.D. | N.D. | N.D. | N.D. |
| Molecular | 2 | N.D. | N.D. | N.D. | N.D. | N.D. |
| Weight | 3 | N.D. | N.D. | N.D. | N.D. | N.D. |
| (HMW) % | 4 | N.D. | N.D. | N.D. | N.D. | N.D. |

As can be seen the polysorbate 80 content did not generate the significant impact on the SEC purity in the condition of with or without agitation. The polysorbate 80 content did not significantly impact the pI, the percentage of main peak, acid peak, and basic peak in cIEF assay in the condition of with or without agitation up to 7 days.

| | | | 3 days | | 7 days | |
|---|---|---|---|---|---|---|
| | | | Non- | | Non- | |
| | | T0 | agitation | 100 rpm | agitation | 100 rpm |
| Main | 1 | 61.5 | 61.4 | 61.4 | 62.4 | 62.3 |
| Peak | 2 | 61.5 | 61.5 | 61.9 | 61.9 | 62.1 |
| % | 3 | 61.2 | 60.8 | 60.5 | 61.5 | 61.6 |
| | 4 | 61.9 | 61.4 | 61.5 | 62 | 61.8 |
| Acid | 1 | 30.9 | 31.1 | 30.5 | 31.1 | 30.6 |
| % | 2 | 31.2 | 31.6 | 30.5 | 31.3 | 31.1 |
| | 3 | 31.5 | 32.2 | 32.3 | 31.5 | 31.6 |
| | 4 | 31.1 | 31.8 | 31.4 | 31.4 | 31.5 |
| Basic | 1 | 7.6 | 7.5 | 8.1 | 6.5 | 7.1 |
| % | 2 | 7.3 | 6.9 | 7.6 | 6.8 | 6.7 |
| | 3 | 7.3 | 7 | 7.2 | 6.9 | 6.8 |
| | 4 | 7 | 6.7 | 7 | 6.7 | 6.7 |

Polysorbate 80 content had no significant impact on the Caliper_Non-reduced purity with or without agitation up to 7 days as shown in the Table below.

| | | 3 days | | 7 days | |
|---|---|---|---|---|---|
| Formu- | | Non- | | Non- | |
| lation | T0 | agitation | 100 rpm | agitation | 100 rpm |

Caliper Non Reduced

| | | | | | | |
|---|---|---|---|---|---|---|
| Purity | 1 | 94.9 | 94.8 | 95 | 94.4 | 94.5 |
| % | 2 | 94.9 | 94.8 | 94.6 | 94.7 | 94.4 |
| | 3 | 94.7 | 94.8 | 94.8 | 94.3 | 94.4 |
| | 4 | 94.9 | 94.8 | 94.6 | 94.3 | 94.3 |

Reduced Caliper analysis was also carried out. PS-80 content had no significant impact on Caliper_Reduced purity with or without agitation in 7 days.

| | | 3 days | | 7 days | |
|---|---|---|---|---|---|
| Formu- | | Non- | | Non- | |
| lation | T0 | agitation | 100 rpm | agitation | 100 rpm |

Caliper Non Reduced

| | | | | | | |
|---|---|---|---|---|---|---|
| Purity | 1 | 99.2 | 99.2 | 99.2 | 99.2 | 99.2 |
| % | 2 | 99.2 | 99.3 | 99.2 | 99.2 | 99.2 |
| | 3 | 99.1 | 99 | 99.2 | 99.2 | 99.2 |
| | 4 | 99.2 | 99.1 | 99.2 | 99.2 | 99.1 |

In order to measure the subvisible particles, around 1500 μL of each sample was taken out from the glass vial container and tested by Micro-Flow Imaging (MFI) according to user's manual. The particle concentration in different size ranges including 1˜2 μm, 2˜5 μm, 5˜10 μm, 10-25 μm and >25 μm were reported (see below). Polysorbate 80 content had no significant impact on particle concentration with or without agitation up to 7 days.

| | | | 3 days | | 7 days | |
|---|---|---|---|---|---|---|
| | Formu- | | Non- | | Non- | |
| | lations | T0 | agitation | 100 rpm | agitation | 100 rpm |
| 1 μm ≤ | 1 | 8318 | NA | 9080 | 1205 | 2046 |
| ECD < | 2 | 25396 | 10831 | 4429 | 7668 | 5898 |
| 2 μm | 3 | 8048 | 2660 | 15877 | 2952 | 4932 |
| | 4 | 8867 | 2590 | 13115 | 9214 | 2728 |
| 2 μm ≤ | 1 | 1792 | 4516 | 2700 | 140 | 574 |
| ECD < | 2 | 6068 | 4215 | 711 | 1137 | 806 |
| 5 μm | 3 | 818 | 363 | 3950 | 927 | 735 |
| | 4 | 1321 | 297 | 3476 | 2418 | 310 |
| 5 μm ≤ | 1 | 369 | NA | 486 | 27 | 52 |
| ECD < | 2 | 770 | 647 | 79 | 237 63 | 63 |
| 10 μm | 3 | 121 | 63 | 379 | 121 | 94 |
| | 4 | 104 | 80 | 253 | 167 | 61 |
| 10 μm ≤ | 1 | 78 | NA | 103 | 2 | 22 |
| ECD < | 2 | 135 | 73 | 7 | 59 | 5 |
| 25 μm | 3 | 39 | 13 | 66 | 20 | 8 |
| | 4 | 14 | 25 | 34 | 16 | 8 |
| ECD ≥ | 1 | 0 | NA | 6 | 0 | 2 |
| 25 μm | 2 | 7 | 14 | 3 | 21 | 15 |
| | 3 | 10 | 10 | 10 | 2 | 2 |
| | 4 | 2 | 6 | 12 | 0 | 12 |

### Example 6

### Addition of Chelator

This study compared the stability of an anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113 in 10 mM L-Histidine buffer (pH=5.8), 0.02% (w/v) polysorbate 80, 10 mM L-Methionine (“L-Met”), 7% w/v sucrose in the presence or absence of 20 uM or 50 uM DTPA.

The three formulations were filled into vials and staged on stability at 5° C. (ambient humidity), 25° C. (60% relative humidity), and 40° C. (75% relative humidity) for eighteen weeks protected from light.

| | | | L-Histidine | | | | | | | |
|---|---|---|---|---|---|---|---|---|---|---|
| Formu- | Anti-TIGIT | | Buffer | | Sucrose | | | Polysorbate 80 | | |
| lation | antibody | | (pH 5.8) | | % (w/v) | L-Met | | % (w/v) | DTPA | |
| 1 | 50 | mg/mL | 10 | mM | 7% | 10 | mM | 0.02% | 0 | |
| 2 | 50 | mg/mL | 10 | mM | 7% | 10 | mM | 0.02% | 20 | uM DTPA |
| 3 | 50 | mg/mL | 10 | mM | 7% | 10 | mM | 0.02% | 50 | uM DTPA |

The colloidal stability of the samples were assessed by size exclusion chromatography (SEC) for purity in which the percentage of monomer was determined, as well as the percentages of high molecular weight species (HMW) and late eluting peaks (LMW species). The UPSEC data to evaluate the levels of % HMW (aggregates), % monomer and % LMW is in the Table below:

| | | 5° C. | | | | 25° C. | | | 40° C. | | | |
|---|---|---|---|---|---|---|---|---|---|---|---|---|
| | Form. | T0 | 4 W | 8 W | 18 W | 4 W | 8 W | 18 W | 2 W | 4 W | 8 W | 18 W |
| % | 1 | 1.54 | N/A | 1.57 | 1.57 | 1.53 | 1.57 | 1.64 | 1.51 | 1.61 | 1.72 | 2.2 |
| HMW | 2 | 1.57 | 1.59 | 1.59 | 1.57 | 1.56 | 1.57 | 1.59 | 1.51 | 1.56 | 1.65 | 1.94 |
| | 3 | 1.61 | 1.61 | 1.61 | 1.58 | 1.56 | 1.57 | 1.59 | 1.53 | 1.57 | 1.63 | 1.91 |
| % | 1 | 98.2 | N/A | 98.2 | 98.2 | 98.1 | 98.0 | 97.8 | 97.9 | 97.6 | 97.2 | 95.7 |
| Monomer | 2 | 98.2 | 98.1 | 98.1 | 98.2 | 98.1 | 98.0 | 97.9 | 98.0 | 97.7 | 97.3 | 96.1 |
| | 3 | 98.1 | 98.1 | 98.1 | 98.2 | 98.1 | 98.0 | 97.9 | 98.0 | 97.7 | 97.4 | 96.2 |
| % | 1 | 0.30 | N/A | 0.28 | 0.22 | 0.36 | 0.42 | 0.54 | 0.55 | 0.82 | 1.11 | 2.07 |
| LMW | 2 | 0.28 | 0.28 | 0.27 | 0.22 | 0.35 | 0.40 | 0.55 | 0.53 | 0.77 | 1.02 | 1.94 |
| | 3 | 0.25 | 0.27 | 0.26 | 0.23 | 0.35 | 0.41 | 0.56 | 0.51 | 0.77 | 1.02 | 1.92 |

As shown in the table above, at 5° C., 25° C. and 40° C., all three formulations showed a trend of increase in % HMW peak and % LMW peak (and a consequent decrease in % monomer peak) for up to 18-week time point. At 25° C., both the formulations showed similar trends, but smaller changes, as compared to 40° C. At 5° C., no substantial changes were observed. Formulation 1 shows a greater increase in % HMW and % LMW as compared to Formulation 2 (20 uM DTPA) and Formulation 3 (50 uM DTPA). Additionally, Formulation 1 showed a greater decrease of % monomer as compared to Formulation 2 and 3. Similar results were seen with HP-IEX analysis (data not shown).

To evaluate if DTPA can protect the formulations from oxidative stress, the three formulations were filled into vials and exposed to light (0.5×ICH and 1×ICH). As seen in the table below, Formulation 1 shows a greater increase in % oxidation of M254, M430 and W104 (the methionines and tryptophan that are susceptible to oxidation) as compared to Formulation 2 (20 uM DTPA) and Formulation 3 (uM DTPA). Thus, DTPA can further improve the stability of the anti-TIGIT antibody formulation.

| | Formulation 1 | Formulation 1 | | Formulation 2 | | Formulation 3 | |
|---|---|---|---|---|---|---|---|
| | Dark | 0.5X | 1X | 0.5X | 1X | 0.5X | 1X |
| | control | ICH | ICH | ICH | ICH | ICH | ICH |
| LC_M4 | 0.2 | 0.2 | 0.2 | 0.2 | 0.2 | 0.2 | 0.2 |
| HC_M34 | 0.3 | 0.3 | 0.3 | 0.3 | 0.3 | 0.3 | 0.4 |
| HC_M81 | 0.2 | 0.2 | 0.2 | 0.2 | 0.2 | 0.2 | 0.3 |
| HC_M254 | 3.6 | 16.3 | 30.2 | 13.7 | 27.5 | 15.9 | 21.5 |
| HC_M430 | 1 | 10.9 | 19.2 | 9.3 | 18.3 | 8.6 | 16.2 |
| HC_W104 | 0.6 | 8.8 | 17.7 | 7.3 | 16.7 | 6.9 | 12.1 |

| | | 5° C. | | | | 25° C. | | | 40° C. | | | |
|---|---|---|---|---|---|---|---|---|---|---|---|---|
| | Form. | T0 | 4 W | 8 W | 18 W | 4 W | 8 W | 18 W | 2 W | 4 W | 8 W | 18 W |
| PS | 1 | 0.24 | 0.23 | 0.23 | 0.22 | 0.20 | 0.18 | 0.16 | 0.19 | 0.17 | 0.16 | 0.13 |
| 80 | 2 | 0.24 | 0.23 | 0.23 | 0.21 | 0.20 | 0.18 | 0.16 | 0.19 | 0.16 | 0.16 | 0.14 |
| Conc. | 3 | 0.22 | 0.22 | 0.22 | 0.21 | 0.19 | 0.17 | 0.15 | 0.18 | 0.16 | 0.15 | 0.13 |

### Example 7

### Long Term Stability of Anti-TIGIT Antibody Formulations

This example describes long term stability data for an anti-TIGIT antibody formulated in an L-histidine buffer, L-methionine, sucrose, polysorbate 80 and water for injection as follows:

| | | Concentration | Quantity |
|---|---|---|---|
| Ingredients | | (mg/mL) | (mg/vial) |
| Active | Anti-TIGIT | 50.0 | 100.0 |
| | antibody | | |
| Inactive | L-Histidine | 0.465 | 0.930 |
| (Excipients) | L-Histidine | 1.47 | 2.94 |
| | Monohydrochloride | | |
| | Monohydrate | | |
| | L-Methionine | 1.49 | 2.98 |
| | Sucrose | 70 | 140.0 |
| | Polysorbate 80 | 0.20 | 0.40 |
| | Water for | NA | Quantity sufficient |
| | Injection | | to 2.0 mL |

The solutions were filled in a USP Type 1 glass vial with elastomeric stopper and aluminum seal. The vials were then incubated at three different storage conditions: 5° C. (ambient humidity), 25° C. (60% relative humidity), and 40° C. (75% relative humidity). Data is collected at time zero, 1 month, 3 months, 6 month for all storage conditions, at 9 months (5° C. and 25° C. storage conditions), 12 months (5° C. and 25° C. storage conditions), 18 months (5° C. storage conditions), 24 months (5° C. storage conditions) and 36 months (5° C. conditions).

### Results

The results demonstrate overall physical and chemical stability of the anti-TIGIT antibody when stored at the recommended long term conditions of 5° C. for 18 months. There was no measurable loss of potency observed and the purity was within specifications under the recommended storage condition. The results are set forth in the following tables:

5° C./ambient Humidity

| Attribute | Time Point (months) | | | | | | |
|---|---|---|---|---|---|---|---|
| Measured | Initial | 1 | 3 | 6 | 9 | 12 | 18 |
| Biological Potency | 96 | 92 | 100 | 94 | 101 | 92 | 99 |
| by Binding ELISA | | | | | | | |
| Purity by UPSEC % | | | | | | | |
| High Molecular | 1.33 | 1.48 | 1.40 | 1.63 | 1.65 | 1.66 | 1.77 |
| Weight Species(%) | | | | | | | |
| Monomer (%) | 98.7 | 98.5 | 98.6 | 98.3 | 98.3 | 98.3 | * |
| Low Molecular | <QL | <QL | <QL | <QL | <QL | <QL | <QL |
| Weight Species (%) | | | | | | | |
| Charge Variants by | | | | | | | |
| HP-IEX % | | | | | | | |
| Acidic Variants | 21.46 | 21.54 | 22.04 | 22.51 | 22.49 | 22.48 | 22.92 |
| Total Main | 68.8 | 68.4 | 67.9 | 67.3 | 67.1 | 67.3 | 67.1 |
| Basic Variants | 9.70 | 10.11 | 10.06 | 10.15 | 10.38 | 10.20 | 9.95 |
| Purity by non- | 96.4 | 96.4 | 96.3 | 96.2 | 96.0 | 95.9 | 95.7 |
| reduced CE-SDS % | | | | | | | |
| Purity by Reduced | 98.1 | 98.2 | 98.0 | 98.1 | 98.0 | 98.1 | 97.7 |
| CE-SDS % | | | | | | | |
| pH | 6.1 | 6.1 | 6.1 | 6.0 | 6.0 | 6.0 | 6.0 |
| Protein | 51.7 | 51.4 | 51.2 | 51.0 | 51.5 | 50.9 | 51.5 |
| Concentration | | | | | | | |
| UV A350 | 0.150 | 0.142 | 0.138 | 0.144 | 0.146 | 0.145 | 0.150 |

QL = Quantitation Limit (0.4%)
* value is 98.2

25° C./60% Relative Humidity

| Attribute | Time Point (months) | | | | | |
|---|---|---|---|---|---|---|
| Measured | Initial | 1 | 3 | 6 | 9 | 12 |
| Biological Potency | 96 | 90 | 94 | 94 | 98 | 95 |
| by Binding ELISA | | | | | | |
| Purity by UPSEC % | | | | | | |
| High Molecular | 1.33 | 1.57 | 1.48 | 1.72 | 1.77 | 1.83 |
| Weight Species(%) | | | | | | |
| Monomer (%) | 98.7 | 98.4 | 98.3 | 97.9 | 97.7 | 97.4 |
| Low Molecular | <QL | <QL | <QL | <QL | 0.57 | 0.77 |
| Weight Species (%) | | | | | | |
| Charge Variants by | | | | | | |
| HP-IEX % | | | | | | |
| Acidic Variants | 21.46 | 23.19 | 28.74 | 34.16 | 39.86 | 44.04 |
| Total Main | 68.8 | 65.8 | 59.7 | 53.8 | 47.6 | 43.5 |
| Basic Variants | 9.70 | 11.06 | 11.51 | 12.02 | 12.55 | 12.44 |
| Purity by non- | 96.4 | 96.0 | 95.2 | 94.4 | 93.2 | 92.1 |
| reduced CE-SDS % | | | | | | |
| Purity by Reduced | 98.1 | 98.0 | 97.8 | 97.4 | 96.7 | 96.1 |
| CE-SDS % | | | | | | |
| pH | 6.1 | 6.1 | 6.1 | 6.0 | 6.0 | 6.0 |
| Protein | 51.7 | 51.1 | 51.1 | 50.9 | 51.5 | 50.9 |
| Concentration | | | | | | |
| UV A350 | 0.150 | 0.150 | 0.163 | 0.179 | 0.196 | 0.209 |

QL = Quantitation Limit (0.4%)

40° C./75% Relative Humidity

| Attribute | Time Point (months) | | | | | |
|---|---|---|---|---|---|---|
| Measured | Initial | 1 | 3 | 6 ^{c} | 9 | 12 |
| Biological Potency | 96 | 96 | 99 | 94 | ND | ND |
| by Binding ELISA | | | | | | |
| Purity by UPSEC % | | | | | | |
| High Molecular | 1.33 | 1.64 | 1.78 | 2.63 | ND | ND |
| Weight Species(%) | | | | | | |
| Monomer (%) | 98.7 | 97.9 | 96.9 | 93.8 | ND | ND |
| Low Molecular | <QL | 0.43 | 1.28 | 3.53 | ND | ND |
| Weight Species (%) | | | | | | |
| Charge Variants by | | | | | | |
| HP-IEX % | | | | | | |
| Acidic Variants | 21.46 | 38.03 | 61.62 | 80.02 | ND | ND |
| Total Main | 68.8 | 47.8 | 25.1 | 10.0 | ND | ND |
| Basic Variants | 9.70 | 14.17 | 13.29 | 9.95 | ND | ND |
| Purity by non- | 96.4 | 93.6 | 88.9 | 79.6 | ND | ND |
| reduced CE-SDS % | | | | | | |
| Purity by Reduced | 98.1 | 96.9 | 94.2 | 87.7 | ND | ND |
| CE-SDS % | | | | | | |
| pH | 6.1 | 6.1 | 6.1 | 6.0 | ND | ND |
| Protein | 51.7 | 52.0 | 51.2 | 51.4 | ND | ND |
| Concentration | | | | | | |
| UV A350 | 0.150 | 0.188 | 0.251 | 0.453 | ND | ND |

QL = Quantitation Limit (0.10%)

### Protein Concentration

Protein concentration stability data for all time points and conditions did not exhibit any noteworthy changes as a function of storage time or condition and all results were within the acceptance criteria of 45-55 mg/ml.

pH

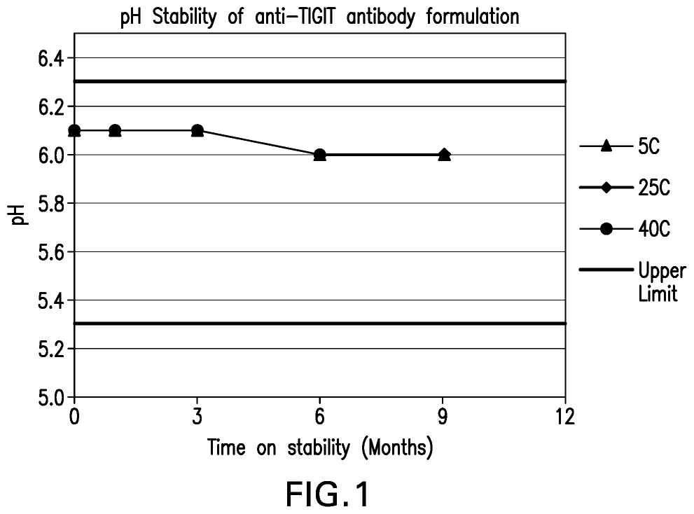

There was no significant change in pH for the 5 C, 25 C, and 40 C conditions. FIG. 1 sets forth the pH data from time point 0 to 9 months.

### Polysorbate 80

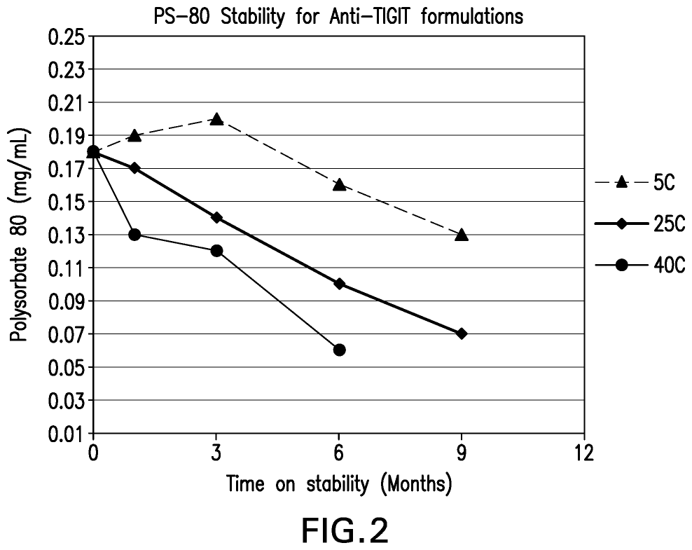

The polysorbate 80 content at the recommended storage condition of 5° C. slightly decreased to 0.13 mg/ml at 9 months and 18 months (18 month data not shown). A decreasing trend in polysorbate 80 was observed at 25° C. (accelerated) and 40° C. (stressed). At 40° C. the polysorbate 80 concentration decreased to 0.06 mg/ml at 6 months and the 25° C. polysorbate 80 content decreased to 0.07 mg/ml at 9 months. The polysorbate 80 concentration data for up to 9 months is set forth in FIG. 2.

### Potency Binding by ELISA

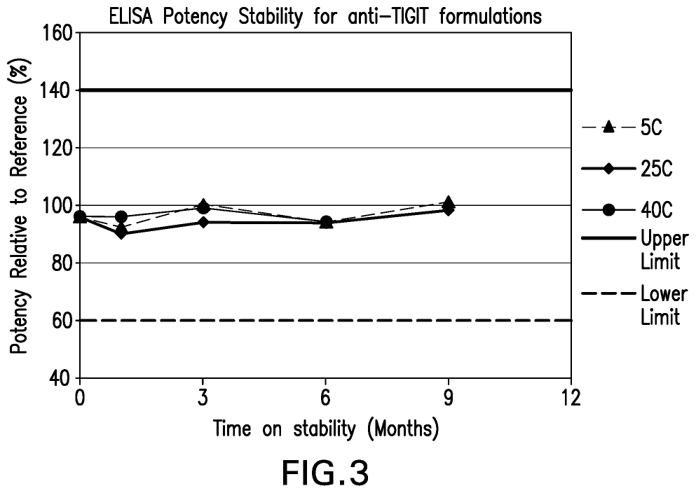

There was no evident trend at any time point or condition in the ELISA results obtained The potency data for up to 9 months is set forth in FIG. 3.

### Purity by UP-SEC

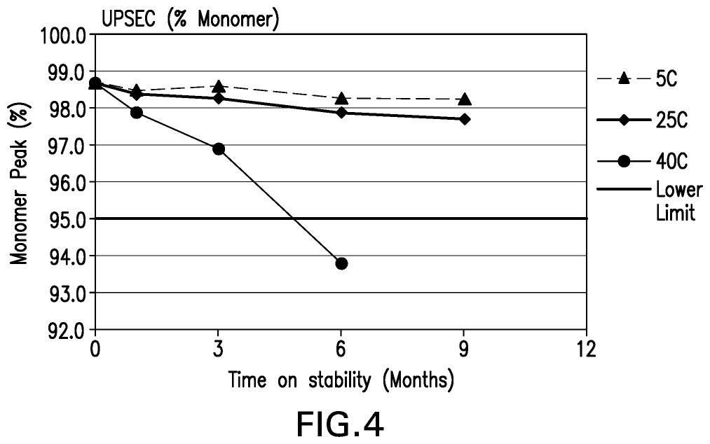

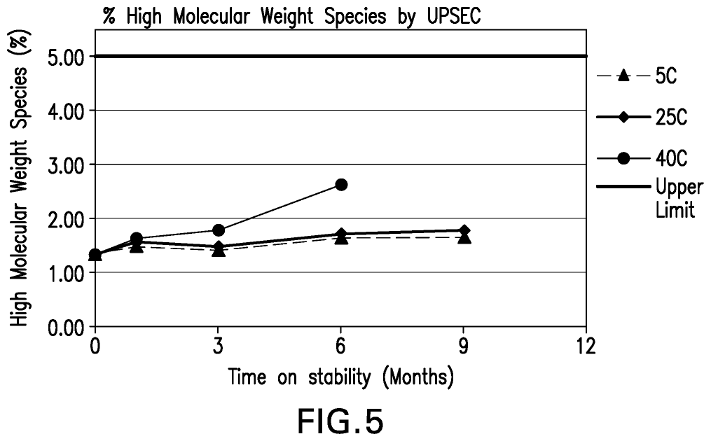

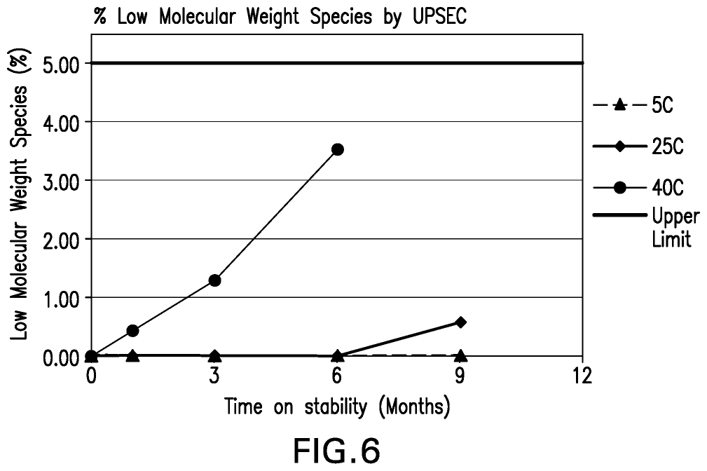

The data for purity by UP-SEC is illustrated below in FIG. 4 for % Monomer, FIG. 5 for % High Molecular Weight species, and FIG. 6 for % Low Molecular Weight species, up to 9 months.

At the recommended storage condition of 5° C., there is a slight decrease in the % Monomer with a corresponding slight increase in % High Molecular Weight species over 18 months of stability. The % Low Molecular Weight Species from Initial to 18 months is below the quantitation limit (<QL) which is equal to 0.4%. At the 25° C. condition, the % Monomer decreased from the Initial to 12 months with a corresponding increase in % High Molecular Weight species. At 9 and 12 months, the % Low Molecular Weight species were reported above the QL.

At the stressed condition of 40° C., % Monomer decreased from 98.7% to 93.8% with corresponding increases in High Molecular Weight species from 1.33% to 2.63% and Low Molecular Weight species from <QL to 3.53%. This result was not unexpected given the nature of the storage condition.

### Reduced and Non-Reduced CD-SDS

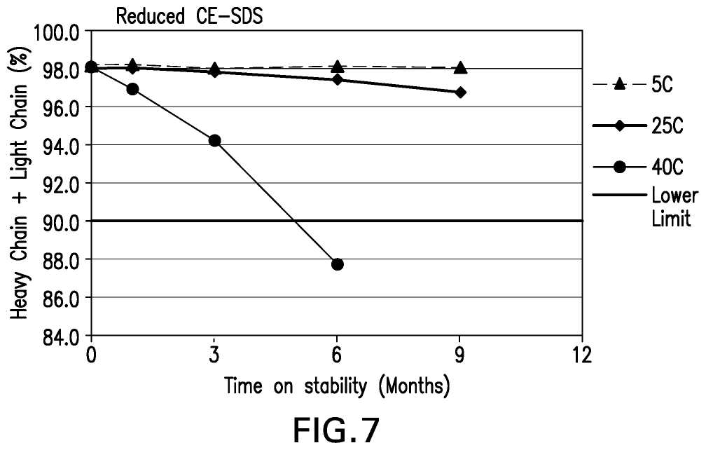

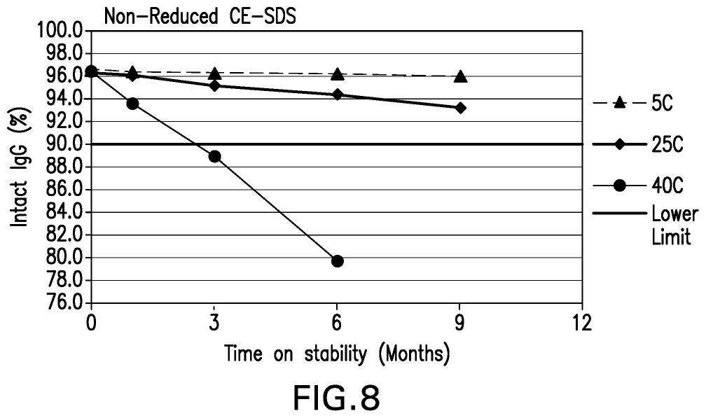

FIGS. 7 and 8 show the purity data up to 9 months as determined by Reduced and Non-reduced CD-SDS. There were no noteworthy trends in Reduced (% heavy and light chain) or Non-reduced (% intact IgG) CE-SDS at the long term storage condition of 5° C. and results were within the GMP drug product acceptance criteria of ≥90.0%. At the accelerated 25° C. condition, a decreasing trend was observed for the Non-reduced condition. For the Reduced CE-SDS condition, a decreasing trend was also observed. For both the Reduced and Non-Reduced CE-SDS all results up to the 9 month time point were within the GMP acceptance criteria. At the 40° C. stressed condition, at 6 months, both the Reduced and Non-Reduced CD-SDS results were below the ≥90.0% acceptance criteria set for the GMP drug product. The Non-Reduced CE-SDS result fell out of specification at 3 months with a result of 88.9% and then further decreased at 6 months to 79.6%. For the Reduced CE-SDS, the % heavy chain and % light chain decreased from 94.2% at 3 months to 87.7% at 6 months. This decrease was not unexpected at 40° C. considering the nature of the condition.

### Charge Variants by HP-IEX

At 5° C. (long term storage), there is a slight increase in % Acidic Variants from the initial at 21.46% to 9 months at 22.49% with a corresponding slight decrease in the Total Main from 68.8% to 67.1% at 9 months. The % Basic Variants begin to slightly increase at 9 months with an increase from 10.15% at 6 months to 10.38% at 9 months. At 25° C. (accelerated), the Total Main decreased from 68.8% at the Initial time point to 47.6% at 9 months. Along with a decrease in the Total Main, a corresponding increase in Acidic Variants was observed from 21.46% to 39.86% and a slight increase in Basic Variants from 9.70% to 11.51%. At 40° C. (stressed), there was a considerable decrease in Total Main to 10.1% at 6 months along with a corresponding considerable increase in Acidic Variants to 80.02% and the Basic Variants to 9.95%.

### Particulate Matter

Particulate matter was measured by mHIAC. Results at the 5° C. condition were well below the acceptance criteria of ≤6000 particles per container for ≥10 μm and ≤600 particles per container for ≥25 μm from the Initial to 9 months. At 25° C., an increase in particles >=10 μm was reported from 13 particles per container at the Initial time point to 460 particles per container at 9 months. There was a decrease in particles for the >=25 μm particulates with a result of 3 particles per container at 9 months. All time points for the 25 C data were within the acceptance criteria for both the >=10 μm and >=25 μm analysis. The data at the 40° C. condition showed a drastic increase in particles >=10 μm with 8258 particles per container at 9 months. This result was outside the acceptance criteria of <=600 particles per container. The result for particles >=25 μm increased at the 9 month stability time point to 124 particles per container meeting the >=25 μm acceptance criteria (<=600 particles per container).

### Turbidity

Turbidity was determined from the spectrophotometric absorbance at 350 nm. At the longer term storage condition 5° C., there was no noteworthy change up to the 9 month time point. At the 25 C condition, there is a slight increase at 3 months with a result of 0.163 AU and it continues to increase to 9 months with a result of 0.196 AU. At 40 C, there is a more pronounced increase starting at 1 month with 0.188 AU and then greatly increasing to 0.453 AU at 9 months.

### Conclusions

Based on the data, at the 18 month testing date, no major changes or trends were observed at the storage condition of 5 C over the course of the stability studies for pH, protein concentration, appearance and visible particles (data not shown) and potency and particulate matter (data not shown). With the exception of a slight increase in color and a decrease in the PS-80 content to 0.13 mg/ml, no noteworthy changes or trends were observed for any stability test at 5° C.

Based on the data for long term stability of 5 C, the anti-TIGIT formulation containing L-histidine buffer, sucrose, polysorbate 80 and L-methionine has an expected shelf life of 30 months. Formulations further comprising a chelator are expected to reduce the degradation of polysorbate 80 which was observed.

### Example 8

### Co-Formulation of an Anti-TIGIT Antibody and an Anti-PD-1 Antibody.

Co-formulation of two antibodies into a single formulation in more convenient for patients and increases compliance with dosing the two antibodies together. Co-formulation of two antibodies into a single formulation in more convenient for patients and increases compliance with dosing the two antibodies together. An anti-TIGIT antibody having the following CDRs: HCDR1 of SEQ ID NO: 108, HCDR2 of SEQ ID NO: 154, HCDR3 of SEQ ID NO: 110, LCDR1 of SEQ ID NO: 111, LCDR2 of SEQ ID NO:112, and LCDR3 of SEQ ID NO: 113 on an IgG1 backbone was co-formulated with pembrolizumab. Based on the protein-protein interactions (shown below), the co-formulation (shown below) were found to be stable across pH 5.0-6.0. Hence, the co-formulation (P1T1) at pH 5.0, 5.5 and 6.0 were chosen and placed on additional thermal stability at 5° C., 25° C. and 40° C. along with the two controls (PD1 antibody and an anti-TIGIT antibody).

| | Pembrolizumab/ | | | | | | |
|---|---|---|---|---|---|---|---|
| | Anti-anti- | | | | | | |
| | TIGIT Ab | | | Anti-TIGIT | | Total | |
| Coformulations | ratio (w/w) | Pembrolizumab | | antibody | | Concentration | |
| P1T1 | 1:1 | 20 | mg/mL | 20 | mg/mL | 40 | mg/mL |

| | | | | | | |
|---|---|---|---|---|---|---|
| P1 (Control) | 1:0 | 20 | mg/mL | None | 20 | mg/mL |

| | | | | | | |
|---|---|---|---|---|---|---|
| T1 (Control) | 0:1 | None | 20 | mg/mL | 10 | mg/mL |

The formulations were prepared as liquid formulations as follows:

| | | | Cryo- | | |
|---|---|---|---|---|---|
| | | | protectant/ | | |
| | | | Tonicity | | |
| Formulation | Buffer | pH | modifier | Surfactant | Antioxidant |
| P1T1 | L-Histidine | 5, 5.5, 6 | Sucrose (7%) | PS-80 | 10 mM L-Met |
| | (10 mM) | | | (0.02%) | |
| P1 (Control) | L-Histidine | 5, 5.5, 6 | Sucrose (7%) | PS-80 | 10 mM L-Met |
| | (10 mM) | | | (0.02%) | |
| T1 (Control) | L-Histidine | 5, 5.5, 6 | Sucrose (7%) | PS-80 | 10 mM L-Met |
| | (10 mM) | | | (0.02%) | |

Each formulation was filled at 1 mL into 2R vials. Stability will be measured by visual inspection, protein concentration, Microwflow Imaging (MFI) (evaluation of particulates), mixed mode size exclusion chromatography (SEC) (evaluation of aggregation, IEX (evaluation of charge variants), and UP-SEC (evaluation of aggregation). The thermal stability protocol is as follows:

| | T0 | 1 month | 2 Month | 3 Month | 5 month | Extra |
|---|---|---|---|---|---|---|
| 5° C. | 1 combo + | 1 combo + | 1 combo + | 1 combo + | 1 combo + | 1 combo + |
| (ambient | 2 mono | 2 mono | 2 mono | 2 mono | 2 mono | 2 mono |
| humidity) | | | | | | |
| 25° C. | | 1 combo + | 1 combo + | 1 combo + | 1 combo + | 1 combo + |
| (60% relative | | 2 mono | 2 mono | 2 mono | 2 mono | 2 mono |
| humidity) | | | | | | |
| 40° C. | | 1 combo + | 1 combo + | 1 combo + | 1 combo + | 1 combo + |
| (75% relative | | 2 mono | 2 mono | 2 mono | 2 mono | 2 mono |
| humidity) | | | | | | |

Protein-protein interactions, which are indicative of colloidal and thermal stability of the different co-formulation was measured. A repulsive protein-protein interaction, as indicated by a positive diffusion interaction parameter (K_{D}) value of K_{D}>0 indicates a stable formulation with low propensity for aggregation. The Kd for the coformulation was found to have a positive K_{D} value which is indicative of repulsive and stabilizing protein-protein interaction which would indicate a lesser propensity to aggregate and a stable coformulation.

Based on the positive diffusion interaction parameter (K_{D}) or K_{D}>0, the antibodies when co-formulated, are expected to behave well when co-formulated together, similar to the single antibody formulations.

1. A formulation comprising:
(i) about 10 mg/ml to about 200 mg/ml of an anti-TIGIT antibody, or antigen binding fragment thereof,
(ii) about 5 mM to about 20 mM buffer;
(iii) about 6% to about 8% weight/volume (w/v) non-reducing sugar;
(iv) about 0.01% to about 0.10% (w/v) non-ionic surfactant; and
(v) about 1 mM to about 20 mM anti-oxidant.

2. The formulation of claim 1, wherein the anti-TIGIT antibody or antigen binding fragment thereof comprises three light chains CDRs comprising CDRL1 of SEQ ID NO: 111, CDRL2 of SEQ ID NO: 112, CDRL3 of SEQ ID NO: 113 and three heavy chain CDRs comprising CDRH1 of SEQ ID NO: 108, CDRH2 of SEQ ID NO: 154, and CDRH3 of SEQ ID NO: 110.

3. The formulation of claim 1, wherein the anti-TIGIT antibody or antigen binding fragment thereof comprises a heavy chain variable region comprising SEQ ID NO: 148 and a light chain variable region comprising SEQ ID NO: 152.

4. The formulation of claim 3, wherein the anti-TIGIT antibody comprises (i) a human heavy chain IgG1 constant domain comprising the amino acid sequence of SEQ ID NO:291 and a human kappa light chain constant domain comprising the amino acid sequence of SEQ ID NO:293; or (ii) a human heavy chain IgG4 constant domain comprising the amino acid sequence of SEQ ID NO:292 and a human kappa light chain constant domain comprising the amino acid sequence of SEQ ID NO:293.

5. The formulation of claim 1, wherein the formulation has a pH between 5.3 and 6.2.

6. The formulation of claim 1, wherein the buffer is a L-histidine buffer, the non-reducing sugar is sucrose, the non-ionic surfactant is polysorbate 80, and the anti-oxidant is L-methionine, the formulation comprising:
(i) about 10 mg/ml to about 200 mg/ml of an anti-TIGIT antibody, or antigen binding fragment thereof;
(ii) about 5 mM to about 20 mM of a L-histidine buffer;
(iii) about 6% to about 8% (w/v) sucrose;
(iv) about 0.01% to about 0.10% (w/v) polysorbate 80; and
(v) about 1 mM to about 20 mM L-methionine.

7. The formulation claim 1,
comprising about 8 mM to about 12 mM of L-histidine buffer; and/or
comprising about 5 mM to about 10 mM L-methionine; and/or
comprising polysorbate 80 at a weight ratio of about 0.02% w/v.

8. (canceled)

9. (canceled)

10. The formulation of claim 1, comprising about 10 mg/ml to about 100 mg/ml of the anti-TIGIT antibody or antigen binding fragment thereof, or wherein concentration of the anti-TIGIT antibody or antigen binding fragment thereof is about 10 mg/ml, 12.5 mg/ml, 25 mg/ml, 50 mg/ml, 75 mg/ml or 100 mg/ml.

11. (canceled)

12. The formulation of claim 1 comprising at least one of the following:
about 25 mg/mL of the anti-TIGIT antibody, 10 mM L-histidine buffer, about 7% w/v sucrose, about 0.02% polysorbate 80, and about 10 mM L-methionine; or
about 50 mg/mL of the anti-TIGIT antibody, 10 mM L-histidine buffer, about 7% w/v sucrose, about 0.02% polysorbate 80, and about 10 mM L-methionine; or
about 75 mg/mL of the anti-TIGIT antibody, 10 mM L-histidine buffer, about 7% w/v sucrose, about 0.02% polysorbate 80, and about 10 mM L-methionine; or
about 100 mg/mL of the anti-TIGIT antibody, 10 mM L-histidine buffer, about 7% w/v sucrose, about 0.02% polysorbate 80, and about 10 mM L-methionine; or
a pH of about 5.5-6.3: or
a pH of about 5.8-6.0: or
a chelator in the formulation.

13. (canceled)

14. (canceled)

15. (canceled)

16. (canceled)

17. (canceled)

18. The formulation of claim 1, further comprising an anti-PD1 antibody or antigen binding fragment thereof.

19. The formulation of claim 18, wherein the anti-human PD-1 antibody or antigen binding fragment thereof comprises three light chain CDRs of SEQ ID NO:1, SEQ ID NO:2 and SEQ ID NO:3 and three heavy chain CDRs of SEQ ID NO:6, SEQ ID NO:7 and SEQ ID NO:8.

20. The formulation of any claim 18, wherein the anti-human PD-1 antibody or antigen binding fragment thereof comprises a variable light region which comprises the amino acid sequence set forth in SEQ ID NO:4, and a variable heavy region which comprises the amino acid sequence set forth in SEQ ID NO:9.

21. The formulation of claim 16, wherein the formulation comprises an anti-human PD-1 antibody that is pembrolizumab.

22. The formulation of claim 18, wherein the ratio of the anti-PD1 antibody to the anti-TIGIT antibody is 1:1.

23. The formulation of claim 18, comprising about 20 mg/ml of the anti-PD1 antibody, about 20 mg/ml of the anti-TIGIT antibody, 10 mM L-histidine buffer, about 7% w/v sucrose, about 0.02% w/v polysorbate 80, and about 10 mM L-methionine.

24. (canceled)

25. The formulation of claim 12, wherein the chelator is diethylenetriaminepentaacetic acid (DTPA).

26. The formulation of claim 1, wherein the formulation is contained in a glass vial or an injection device.

27. The formulation of claim 1 that is a liquid formulation, that is frozen to at least below −70° C., or is a reconstituted solution from a lyophilized formulation.

28. The formulation of claim 1, wherein after 12 months at 5° C.:
(i) the % monomer of the anti-TIGIT antibody is ≥95% as determined by size exclusion chromatography;
(ii) the % heavy chain and light chain of the anti-TIGIT antibody is ≥90% as measured by reduced CE-SDS;
(iii) the % heavy chain and light chain of the anti-TIGIT antibody is ≥95% as measured reduced CE-SDS;
(iv) the % intact IgG of the anti-TIGIT antibody is ≥90% as measured by non-reduced CE-SDS; and/or
(v) % intact IgG of the anti-TIGIT antibody is ≥95% as measured by non-reduced CE-SDS.

29. A method of treating cancer or chronic infection in a human patient in need thereof, the method comprising administering an effective amount of the formulation of claim 1.

30. (canceled)
